(19) United States
(12) Patent Application Publication
(10) Pub. No.: US 2024/0350494 A1
(43) Pub. Date: Oct. 24, 2024

(54) THERAPEUTIC MODULATION OF INTEGRINS

(71) Applicant: Pliant Therapeutics, Inc., South San Francisco, CA (US)

(72) Inventors: Scott TURNER, Oakland, CA (US); Eric LEFEBVRE, San Francisco, CA (US); Gregory P. COSGROVE, Centennial, CO (US); Martin L. DECARIS, Alameda, CA (US)

(21) Appl. No.: 18/688,267

(22) PCT Filed: Sep. 1, 2022
(86) PCT No.: PCT/US22/75954

§ 371 Date: Feb. 29, 2024

Related U.S. Application Data
(60) Provisional application No. 63/240,862, filed on Sep. 3, 2021.

(51) Int. Cl. A61K 31/517; A61K 49/00; A61P 11/00

(52) U.S. Cl. CPC A61K 31/517; A61K 49/0004; A61P 11/00

(57) ABSTRACT

The invention relates methods of determining therapeutic doses for conditions mediated by integrin and methods of therapy for conditions mediated by integrin comprising administering an integrin modulating compound in an amount that achieves receptor occupancy of the integrin. The invention further relates to dosage forms for daily administration of integrin modulating compounds that are useful for treating conditions mediated by at least one integrin including, e.g., fibrosis such as idiopathic pulmonary fibrosis (IFF) and nonspecific interstitial pneumonia (NSIP).

### CROSS-REFERENCE TO RELATED APPLICATIONS

This application claims priority benefit of U.S. Provisional Patent Application No. 63/240,862 filed Sep. 3, 2021, the content of which is hereby incorporated by reference herein in its entirety.

### BACKGROUND OF THE INVENTION

Fibrosis, a pathologic feature of many diseases, is caused by a dysfunction in the body's natural ability to repair damaged tissues. If left untreated, fibrosis can result in scarring of vital organs causing irreparable damage and eventual organ failure.

Patients with nonalcoholic fatty liver disease (NAFLD) may progress from simple steatosis to nonalcoholic steatohepatitis (NASH) and then fibrosis. While liver fibrosis is reversible in its initial stages, progressive liver fibrosis can lead to cirrhosis.

Fibrosis in the kidney, characterized by glomerulosclerosis and tubulointerstitial fibrosis, is the final common manifestation of a wide variety of chronic kidney diseases (CKD).

Irrespective of the initial causes, progressive CKD often results in widespread tissue scarring that leads to destruction of kidney parenchyma and end-stage renal failure, a devastating condition that requires dialysis or kidney replacement.

Scleroderma encompasses a spectrum of complex and variable conditions primarily characterized by fibrosis, vascular alterations, and autoimmunity. The scleroderma spectrum of disorders share the common feature of fibrosis, resulting in hardening or thickening of the skin.

For some patients, this hardening occurs only in limited areas, but for others, it can spread to other major organs.

Following myocardial infarction, cardiac structural remodeling is associated with an inflammatory reaction, resulting in scar formation at the site of the infarction. This scar formation is a result of fibrotic tissue deposition which may lead to reduced cardiac function and disruption of electrical activity within the heart.

Crohn's Disease is a chronic disease of unknown etiology tending to progress even in the setting of medical or surgical treatment. Intestinal fibrosis is among the most common complications of Crohn's disease, resulting in stricture formation in the small intestine and colon.

Idiopathic pulmonary fibrosis (IPF) is a chronic, progressive, fibrosing disease of unknown etiology, occurring in adults and limited to the lungs. In IPF, the lung tissue becomes thickened, stiff, and scarred. As lung fibrosis progresses, it becomes more difficult for the lungs to transfer oxygen into the bloodstream and the organs do not receive the oxygen needed to function properly. IPF currently affects approximately 200,000 people in the U.S., resulting in 40,000 deaths per year. Patients diagnosed with IPF experience progressive breathlessness and eventually, complete respiratory failure.

Primary biliary cholangitis (PBC), also known as primary biliary cirrhosis, is a chronic disease of the liver that causes damage and fibrosis in the liver. It results from a slow, progressive destruction of the small bile ducts of the liver, causing bile and other toxins to build up in the liver, a condition called cholestasis. Over time, this leads to scarring and fibrosis in both the liver and biliary tract.

Nonspecific interstitial pneumonia (NSIP) is a rare disorder that affects the tissue that surrounds and separates the tiny air sacs of the lungs. These air sacs, called the alveoli, are where the exchange of oxygen and carbon dioxide takes place between the lungs and the bloodstream. Interstitial pneumonia is a disease in which the mesh-like walls of the alveoli become inflamed. The pleura (a thin covering that protects and cushions the lungs and the individual lobes of the lungs) might become inflamed as well. There are two primary forms of NSIP—cellular and fibrotic. The cellular form is defined mainly by inflammation of the cells of the interstitium. The fibrotic form is defined by thickening and scarring of lung tissue. This scarring is known as fibrosis and is irreversible. When the lung tissue thickens or becomes scarred, it does not function as effectively. Breathing becomes less efficient, and there are lower levels of oxygen in the blood. (Kim et al., Proc. Am. Thorac. Soc. (2006) 3:285-292; Lynch, D., Radiology (2001) 221:583-584; Kinder et al., Am. J. Respir. Crit. Care Med. (2007) 176:691-697)

Available courses of treatment are scarce, as there are currently no options on the market proven to have an effect on long-term patient survival or symptomatology. For example, agents such as pirfenidone and nintedanib have been studied for treatment of fibrosis. In the treatment of IPF, pirfenidone and nintedanib have been used, but have shown less therapeutic efficacy than desired while also exhibiting numerous side effects. There remains a need for treatment of fibrotic diseases.

The αvβ6 integrin is expressed in epithelial cells, and binds to the latency-associated peptide of transforming growth factor-β1 (TGFβ1) and mediates TGFβ1 activation. Its expression level is significantly increased after injury to lung and cholangiocytes, and plays a critical in vivo role in tissue fibrosis. Increased levels are also associated with increased mortality in IPF and NSIP patients.

Primary sclerosing cholangitis (PSC) involves bile duct inflammation, and fibrosis that obliterates the bile ducts. The resulting impediment to the flow of bile to the intestines can lead to cirrhosis of the liver and subsequent complications such as liver failure and liver cancer. Expression of αvβ6 is elevated in liver and bile duct of PSC patients.

The present disclosure provides for αvβ6 integrin inhibitors that may be useful for treatment of fibrosis.

### BRIEF SUMMARY OF THE INVENTION

Disclosed are integrin binders and inhibitors, compositions containing these compounds and methods for treating diseases mediated by integrin such as a fibrotic disease.

In one aspect, provided is a method for determining a therapeutic dose of a compound, or a pharmaceutically acceptable salt thereof, for treating a condition mediated by at least one integrin, comprising: (a) administering the compound, or a pharmaceutically acceptable salt thereof, to a subject in need of therapy for the condition, wherein the subject expresses the at least one integrin and the compound, or pharmaceutically acceptable salt thereof, binds to the at least one integrin; (b) measuring the percent occupancy of the compound in the at least one integrin; and (c) determining the amount of the compound, or a pharmaceutically acceptable salt thereof, that is effective to achieve a predetermined percent occupancy of the at least one integrin, wherein the amount of compound or pharmaceutically acceptable salt thereof that is required to achieve the predetermined percent occupancy is the therapeutic dose of the compound or a pharmaceutically acceptable salt thereof. In some embodiments, the subject has a tissue involved in the condition that expresses the at least one integrin, and the at least one integrin is in the tissue of the subject. In some embodiments, the subject expresses the at least one integrin in the tissue of the subject. In some embodiments, the compound is an integrin binding compound. In some embodiments, the compound is an integrin inhibitor.

In some embodiments, (a) and (b) are performed on a first subject and one or more additional subjects, wherein the first and the one or more additional subjects have the same condition, and wherein the first subject is administered a first amount of the compound or a pharmaceutically acceptable salt thereof and the one or more additional subjects are each administered an amount of the compound or a pharmaceutically acceptable salt thereof that is different than the amount given to the first subject. In some embodiments, the first and one or more additional subjects can together be a cohort of subjects, such as 3 or 4 or 5 or 10 or 15 or more human subjects.

In some embodiments, the at least one integrin is α_{V}β₁ integrin or α_{V}β₆ integrin. In some embodiments, the at least one integrin is α_{V}β₁ integrin and α_{V}β₆ integrin.

In some embodiments, the % occupancy is measured at about 4 hours following administration of the compound. In some embodiments, percent occupancy is measured using data obtained from a PET/CT scan of the subject after administration of the compound, or a pharmaceutically acceptable salt thereof.

In some embodiments, the subject is a human. In some embodiments, the condition is IPF or PSC. In some embodiments, the condition is IPF. In some embodiments, the condition is PSC.

In some embodiments, the compound is an integrin binding compound. In some embodiments, the compound is an integrin inhibitor. In some embodiments, the compound is a small molecule, protein, or antibody. In some embodiments, the compound is a small molecule.

In some embodiments, the compound is a protein. In some embodiments, the compound is an antibody. In some embodiments, the compound is compound 5 or a salt thereof, such as a phosphate salt or any pharmaceutically acceptable form thereof, including, e.g., polymorphs of Form I, Form II, Form III, or Form IV.

In some embodiments, the tissue comprises blood, lung tissue, liver tissue, skin tissue, cardiac tissue, kidney tissue, gastrointestinal tissue, gall bladder tissue, or bile duct tissue. In some embodiments, the tissue comprises lung tissue, liver tissue, skin tissue, cardiac tissue, kidney tissue, gastrointestinal tissue, gall bladder tissue, or bile duct tissue. In some embodiments, the tissue comprises lung, liver, skin, heart, kidney, or gastrointestinal tissue. In some embodiments, the tissue comprises lung tissue or liver tissue. In some embodiments, the tissue comprises lung tissue. In some embodiments, the tissue comprises liver tissue.

In another aspect, provided is a method for determining the therapeutically effective percent occupancy of an integrin of a compound that binds to the integrin, or a pharmaceutically acceptable salt thereof, the method comprising:
(a) administering the compound, or a pharmaceutically acceptable salt thereof, to a subject having a condition mediated by at least one integrin and the subject expresses the at least one integrin;
(b) determining the percent occupancy of the compound in at least one integrin in the subject; and
(c) measuring in the subject one or more parameters associated with the condition before and after administration of the compound, or a pharmaceutically acceptable salt thereof,
   wherein a beneficial effect on one or more parameters associated with the condition measured after administration of the compound, or pharmaceutically acceptable salt thereof, compared to measurement of the same parameter in the subject before administration of the compound, or a pharmaceutically acceptable salt thereof, indicates a therapeutically effective percent occupancy of the integrin. In some embodiments, the subject has a tissue that expresses the at least one integrin. In some embodiments, the method comprises determining the percent occupancy of the compound in at least one integrin in the tissue of the subject. In some embodiments, the compound is an integrin binding compound. In some embodiments, the compound is an integrin inhibitor.

In some embodiments, the at least one integrin is α_{V}β₁ integrin or α_{V}β₆ integrin. In some embodiments, the at least one integrin is α_{V}β₁ integrin and α_{V}β₆ integrin.

In some embodiments, the % occupancy is measured at about 4 hours following administration of the compound. In some embodiments, percent occupancy is measured using data obtained from a PET/CT scan of the subject after administration of the compound, or a pharmaceutically acceptable salt thereof.

In some embodiments, the subject is a human. In some embodiments, the condition is IPF or PSC. In some embodiments, the condition is IPF. In some embodiments, the condition is PSC.

In some embodiments, the compound is compound 5 or a salt thereof, such as a phosphate salt or any pharmaceutically acceptable form thereof, including, e.g., polymorphs of Form I, Form II, Form III, or Form IV.

In some embodiments, the tissue comprises blood, lung, liver, skin, heart, kidney, or gastrointestinal tissue. In some embodiments, the tissue comprises lung, liver, skin, heart, kidney, or gastrointestinal tissue. In some embodiments, the tissue is lung tissue or liver tissue. In some embodiments, the tissue is lung tissue. In some embodiments, the tissue is liver tissue.

In some embodiments, the parameters include levels of at least one integrin. In some embodiments, the include levels of at least one integrin comprising a β₆ subunit. In some embodiments, the parameters include serum markers of cholestasis, concurrent clinical or histologic features of inflammatory bowel disease (IBD), and/or histologic features compatible with PSC. In some embodiments, the parameters include forced vital capacity (FVC), HRCT-based Quantitative Lung Fibrosis score (QLF), alveolar pSmad2/Smad2 percentage, or serum biomarkers of collagen synthesis. In some embodiments, the parameters are measured by high-resolution computed tomography (HRCT), BAL with or without transbronchial lung cryobiopsy (TBLC), surgical lung biopsy (SLB), magnetic resonance cholangiopancreatography (MRCP), endoscopic retrograde cholangiography (ERC).

In one aspect, provided is a method of treating a fibrotic disease in an individual (such as a human) in need thereof comprising administering to the individual a therapeutically effective amount of an integrin modulating compound, such as compound 5, or or a pharmaceutically acceptable salt thereof. In some embodiments, the compound is an integrin binding compound. In some embodiments, the compound is an integrin inhibitor. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC). In some embodiments, the fibrotic disease is pulmonary fibrosis (such as IPF), liver fibrosis, skin fibrosis, psoriasis, scleroderma, cardiac fibrosis, renal fibrosis, gastrointestinal fibrosis, primary sclerosing cholangitis, or biliary fibrosis (such as PBC). In some embodiments, the fibrotic disease is pulmonary fibrosis (such as IPF). In some embodiments, the fibrotic disease is liver fibrosis. In some embodiments, the fibrotic disease is skin fibrosis. In some embodiments, the fibrotic disease is psoriasis. In some embodiments, the fibrotic disease is scleroderma. In some embodiments, the fibrotic disease is cardiac fibrosis. In some embodiments, the fibrotic disease is renal fibrosis. In some embodiments, the fibrotic disease is gastrointestinal fibrosis. In some embodiments, the fibrotic disease is primary sclerosing cholangitis. In some embodiments, the fibrotic disease is biliary fibrosis (such as PBC).

In another aspect, provided is a method of delaying the onset and/or development of a fibrotic disease in an individual (such as a human) who is at risk for developing a fibrotic disease comprising administering to the individual a therapeutically effective amount of an integrin modulating compound, such as compound 5, or or a pharmaceutically acceptable salt thereof. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or PBC. In some embodiments, the fibrotic disease is pulmonary fibrosis (such as IPF), liver fibrosis, skin fibrosis, psoriasis, scleroderma, cardiac fibrosis, renal fibrosis, gastrointestinal fibrosis, primary sclerosing cholangitis, or biliary fibrosis (such as PBC). In some embodiments, the fibrotic disease is psoriasis. In some embodiments, the individual at risk of developing a fibrotic disease has or is suspected of having NAFLD, NASH, CKD, scleroderma, Crohn's Disease, NSIP, PSC, PBC, or is an individual who has had or is suspected of having had a myocardial infarction. In some embodiments, the individual at risk of developing a fibrotic disease has or is suspected of having psoriasis. In some embodiments, the compound is an integrin binding compound. In some embodiments, the compound is an integrin inhibitor.

In another aspect, provided is a method of treating a fibrotic disease in an individual (such as a human) in need thereof comprising administering to the individual a therapeutically effective amount of an integrin modulating compound, such as compound 5, or or a pharmaceutically acceptable salt thereof. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC). In some embodiments, the fibrotic disease is pulmonary fibrosis (such as IPF), liver fibrosis, skin fibrosis, psoriasis, scleroderma, cardiac fibrosis, renal fibrosis, gastrointestinal fibrosis, primary sclerosing cholangitis, or biliary fibrosis (such as PBC). In some embodiments, the fibrotic disease is psoriasis. In some embodiments, the compound is an integrin binding compound. In some embodiments, the compound is an integrin inhibitor.

In another aspect, provided is a method of delaying the onset and/or development of a fibrotic disease in an individual (such as a human) who is at risk for developing a fibrotic disease comprising administering to the individual a therapeutically effective amount of an integrin modulating compound, such as compound 5, or or a pharmaceutically acceptable salt thereof. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or PBC. In some embodiments, the fibrotic disease is pulmonary fibrosis (such as IPF), liver fibrosis, skin fibrosis, psoriasis, scleroderma, cardiac fibrosis, renal fibrosis, gastrointestinal fibrosis, primary sclerosing cholangitis, or biliary fibrosis (such as PBC). In some embodiments, the fibrotic disease is psoriasis. In some embodiments, the individual at risk of developing a fibrotic disease has or is suspected of having NAFLD, NASH, CKD, scleroderma, Crohn's Disease, NSIP, PSC, PBC, or is an individual who has had or is suspected of having had a myocardial infarction. In some embodiments, the individual at risk of developing a fibrotic disease has or is suspected of having psoriasis.

Further provided is a kit comprising an integrin modulating compound, such as compound 5, or a pharmaceutically acceptable salt thereof. In some embodiments, the kit comprises instructions for use according to a method described herein, such as a method of treating a fibrotic disease in an individual.

In another aspect, provided is a method of treating a condition mediated by at least one integrin comprising providing a subject in need of therapy, the subject comprising: the condition mediated by the at least one integrin, and a tissue involved in the condition that expresses the at least one integrin; and modulating the at least one integrin in the tissue in the subject effective to treat the condition, comprising administering to the subject at least one compound that binds to a receptor of the at least one integrin, the at least one compound being administered to the subject in an amount effective to achieve a percent occupancy of the receptor of the at least one integrin in the tissue of the subject of at least about 50%. In some embodiments, the compound is an integrin binding compound. In some embodiments, the compound is an integrin inhibitor. In some embodiments, the at least one integrin mediates a fibrotic disease or psoriasis. In some embodiments, the fibrotic disease is idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, scleroderma, diabetic nephropathy, diabetic kidney disease, focal segmental glomerulosclerosis, chronic kidney disease, and Crohn's Disease. In some embodiments, the fibrotic disease is psoriasis.

### BRIEF DESCRIPTION OF THE FIGURES

FIG. 1A is a graph showing that compound 5 and the selective antibody α_{V}β₆ inhibitor 3G9 both substantially inhibited normal bronchial epithelial cell adhesion to LAP, in contrast with the α_{V}β₁-selective small molecule inhibitor.

FIG. 1B shows that compound 5 and the α_{V}β₁-selective small molecule inhibitor both substantially inhibited cell adhesion in the IPF-derived lung fibroblasts, in contrast to the selective antibody α_{V}β₆ inhibitor, 3G9.

FIG. 2A is a graph of PSMAD3/SMAD3 in lung tissue from healthy mice administered PBS vehicle and varying levels of compound 5 for 4 days.

FIG. 2B is a graph of PSMAD3/SMAD3 in BALF drawn from the same healthy mice administered PBS vehicle and varying levels of compound 5 for 4 days.

FIG. 2C is a graph showing that compared to the healthy mice, lung tissue in the vehicle-treated mice experienced a substantial increase in SMAD3 phosphorylation.

FIG. 2D is a graph showing that compared to the healthy mice, lung tissue in the vehicle-treated mice experienced a substantial accumulation of new collagen as evidenced by the percentage of lung collagen containing ²H-labeled hydroxyproline.

FIG. 2E shows that compared to the healthy mice, the vehicle-treated mice experienced a significant increase in total pulmonary collagen, as measured by μg of hydroxyproline.

FIG. 2F is a high resolution second harmonic generation image of fibrillar collagen (collagen type I and III) taken from formalin-fixed paraffin embedded lung tissue sections from a healthy mouse lung.

FIG. 2G is a high resolution second harmonic generation image of fibrillar collagen (collagen type I and III) taken from formalin-fixed paraffin embedded lung tissue sections from a vehicle-treated mouse lung.

FIG. 2H is a high resolution second harmonic generation image of fibrillar collagen (collagen type I and III) taken from formalin-fixed paraffin embedded lung tissue sections from a test-article treated mouse lung (500 mg/kg BID of compound 5).

FIG. 2I is a graph showing the percent total collagen area in the second harmonic generation mouse lung images of FIGS. 2F, 2G, and 2H.

FIG. 2J is a graph of sequential measurements in bleomycin-treated mice, which demonstrated a close inverse relationship between pSMAD3 levels in lung vs. plasma drug exposure.

FIG. 2K is a graph of sequential measurements in bleomycin-treated mice, which demonstrated a close inverse relationship between pSMAD3 levels in BALF cells vs. plasma drug exposure.

FIG. 3A is a bar graph, normalized to control slices treated with DMSO, showing that all test treatments reduced Type I Collagen gene Col1a1 expression.

FIG. 3B is a bar graph, normalized to control slices treated with DMSO, showing that all test treatments reduced lung Col1a1 expression.

FIG. 4A is a bar graph showing that compared to the DMSO vehicle control slices, both nintedanib and pirfenidone showed a slight increase in lung Col1a1 expression.

FIG. 4B is a bar graph showing the concentration of compound needed to reduce lung slice Col1a1 expression by 50% compared to DMSO control slices.

FIG. 4C is a bar graph, normalized to control slices treated with DMSO, showing that all test treatments reduced lung Col1a1 expression.

FIG. 4D is a bar graph showing relative expression of COL1A1 in precision cut lung slices (PCLS) from idopathic pulmonary fibrosis (IPF) lung tissue upon exposure to Compound 5, clinical standard of care compounds nintedanib (Nin) and pirfenidone (Pirf), and an ALK5 inhibitor, all versus DMSO control.

FIG. 4E is a bar graph showing a dose dependent reduction of COL1A1 expression in PCLS from human IPF lung tissue upon treatment with concentrations of compound 5 ranging from 200 μM to 1 μM. COL1A1 expression is also graphed for the PCLS in the presence of 0.1% DMSO control, and an Alk5 inhibitor at 1 μM.

FIG. 4F is a bar graph showing the effect of dual selective α_{V}β₆ and α_{V}β₁ inhibition (Compound 5 at 1.82 μM) on the ratio of pSMAD2/SMAD2 in PCLS from human IPF lung tissue samples. The ratio of pSMAD2/SMAD2 is also graphed for the PCLS in the presence of 0.1% DMSO control, and an Alk5 inhibitor at 1 μM

FIG. 5A shows single ascending dose (SAD) study data for administration of 15, 30, 50, and 75 mg of Compound 5.

FIG. 5B shows the multiple ascending dose (MAD) study data for administration of 10, 20, and 40 mg of Compound 5.

FIGS. 6A-6F are a series of graphs showing data for subjects administered 40 mg/day of the selected integrin inhibitor (compound 5). The data in FIGS. 6A-6F include the blood plasma concentration (“PK”, round dots) of the administered integrin inhibitor and the relative change in pSMAD2:SMAD2 ratio from baseline (Day −1) in BAL (bronchoalveolar lavage) samples (“pSMAD”, square dots) through the displayed time course (hours) subsequent to the dose of inhibitor administered on Day 7. The peak of the blood plasma concentration (“PK” curve) is recorded as Cₘₐₓ.

FIG. 6G shows the % change in BAL SMAD2 phosphorylation levels (pSMAD2:SMAD2 ratio) on Day 7 compared to baseline levels recorded on Day −1, for subjects receiving placebo treatment, and subjects in which the Cₘₐₓ of the integrin inhibitor was measured to be less than 700 ng/mL, from 700 ng/mL to 900 ng/mL, and greater than 900 ng/mL.

FIG. 6H shows the % change in SMAD2 phosphorylation (pSMAD2:SMAD2 ratio) (all timepoints) correlated with Cₘₐₓ in subjects administered a 40 mg dose of Compound 5) compared to baseline levels recorded on Day −1.

FIG. 7 is a graph of unbound plasma concentration (X-axis) vs Vt (Y-axis) for the baseline Vt at each dose, the measured Vt after each dose, and a fit line.

FIG. 8 is a graph of unbound plasma concentration (X-axis) vs % receptor occupancy (Y-axis).

FIG. 9 is a bar chart showing % target engagement for each subject and dose.

FIG. 10 describes dose dependent effects of Compound 5.

FIG. 11 describes study design and objectives.

FIG. 12 describes study participant disposition.

FIG. 13 describes baseline demographics of the study participants.

FIG. 14 describes baseline disease characteristics of the study participants.

FIG. 15 summarizes overall safety of the study.

FIG. 16 summarizes overall safety of the study with and without background Standard of Care (SoC).

FIG. 17 describes the most frequent treatment emergent adverse events (TEAEs).

FIG. 18 illustrates that no treatment-emergent serious adverse events (SAEs) were observed with Compound 5.

FIG. 19 illustrates the change in FVC (forced vital capacity) from baseline to Week 12.

FIG. 20 illustrates the change in FVC over time in pooled Compound 5 groups.

FIG. 21 illustrates the change in FVC over time in the 40 mg Compound 5 group.

FIG. 22 illustrates the change in FVC over time in the 80 mg Compound 5 group.

FIG. 23 illustrates the change in FVC over time in the 160 mg Compound 5 group.

FIG. 24 illustrates the change in FVC from baseline to Week 12 in the subgroup on Standard of Care.

FIG. 25 illustrates the change in FVC from baseline to Week 12 in the subgroup not on Standard of Care.

FIG. 26 illustrates the proportion of participants with forced vital capacity-% predicted (FVCpp) decline greater than or equal to 10%.

FIG. 27 compares serum biomarker of collagen synthesis (PRO—C3 Type III collagen synthesis neoepitope) in Compound 5 groups versus placebo groups.

FIG. 28 compares serum biomarker of collagen synthesis (PRO—C6 TypeVI collagen synthesis neoepitope) in Compound 5 groups versus placebo groups.

FIG. 29 illustrates the Mean Percent Change in Quantitative Lung Fibrosis (the extent from baseline to week 12 in the CT Protocol Population), using High-Resolution Computed Tomography (HRCT) based Quantitative Lung Fibrosis (QLF) imaging.

FIG. 30 illustrates the Mean Percent Change in Quantitative Lung Fibrosis (the extent from baseline to week 12) in the CT Protocol Population within Screening Window, using High-Resolution Computed Tomography (HRCT) based Quantitative Lung Fibrosis (QLF) imaging.

### DETAILED DESCRIPTION OF THE INVENTION

The present disclosure provides, inter alia, compounds of formula (A), and variations thereof, or a salt thereof, pharmaceutical compositions comprising compounds of formula (A) or a salt thereof, and methods of using such compounds and compositions in treating fibrotic diseases.

The present disclosure provides, inter alia, compounds of formula (I), and variations thereof, or a salt thereof, pharmaceutical compositions comprising compounds of formula (I) or a salt thereof, and methods of using such compounds and compositions in treating fibrotic diseases.

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. Beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. Also encompassed by “treatment” is a reduction of pathological consequence of fibrosis. The methods of the invention contemplate any one or more of these aspects of treatment.

As used herein, the term “effective amount” intends such amount of a compound of the invention which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

A “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like) and formulating the mixture according to the desired route of delivery (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

Unless otherwise stated, “substantially pure” intends a composition that contains no more than 10% impurity, such as a composition comprising less than 9%, 7%, 5%, 3%, 1%, 0.5% impurity.

The terms “% target engagement” and “percent occupancy of the receptor” are used synonymously, to refer to the percentage of the receptor that is occupied.

It is understood that aspects and embodiments described herein as “comprising” include “consisting of” and “consisting essentially of” embodiments.

### Compounds

In one aspect, the compound is an integrin modulating compound. In some embodiments, the compound is an integrin binding compound. In some embodiments, the compound is an integrin inhibitor. In some embodiments, the compound is a small molecule, protein, or antibody. In some embodiments, the compound is a small molecule. In some embodiments, the compound is a protein.

Representative integrin modulating compounds are disclosed in, e.g., U.S. Pat. Nos. 10,793,564 and 11,419,869, each herein incorporated by reference in their entirety.

In some embodiments, the integrin modulating compound is compound 5:

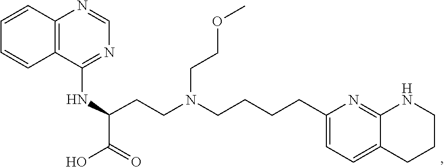 [SMILES: COCCN(CCCCc1ccc2c(n1)NCCC2)CC[C@H](Nc1ncnc2ccccc12)C(=O)O]

or a pharmaceutically acceptable salt thereof. In some embodiments, the integrin modulating compound is compound 5. The chemical name of compound 5 is (S)-4-((2-methoxyethyl)(4-(5,6,7,8-tetrahydro-1,8-naphthyridin-2-yl)butyl)amino)-2-(quinazolin-4-ylamino)butanoic acid, which is disclosed in U.S. Pat. Nos. 10,793,564 and 11,419,869, each of which is herein incorporated by reference in its entirety.

Polymorphic forms of compound 5, including phosphate (Form I), fumarate (Form II), and 1,5-naphthalenedisulfonate salt (Form III) salts, and a mixed solvate of isopropyl alcohol and water phosphate salt (Form IV) are disclosed in U.S. patent application Ser. No. 17/531,074, published as U.S. Patent Publication No. 2022-0117468-A1, each of which is herein incorporated by reference in its entirety. In some embodiments, the compound is an antibody. In some embodiments, the compound is compound 5 or a salt thereof, such as a phosphate salt or any pharmaceutically acceptable form thereof, including, e.g., polymorphs of Form I, Form II, Form III, or Form IV. In some embodiments, compound 5 is a crystalline form of a salt or solvate thereof. In some embodiments, compound 5 is a phosphate, fumarate, 1,5-naphthalenedisulfonate, or a mixed solvate of isopropyl alcohol and water phosphate salt. In some embodiments, compound 5 is a phosphate salt. In some embodiments, compound 5 is a fumarate salt. In some embodiments, compound 5 is a 1,5-naphthalenedisulfonate salt. In some embodiments, compound 5 is a mixed solvate of isopropyl alcohol and water phosphate salt.

Crystalline forms of compound 5 exhibit X-ray powder diffraction (XRPD) patterns (U.S. patent application Ser. No. 17/531,074, published as U.S. Patent Publication No. 2022-0117468-A1, each of which is herein incorporated by reference in its entirety). In some embodiments, the phosphate salt of compound 5 is Form I, characterized as having an XRPD pattern comprising: (a) peaks at angles 2-theta of about 4.31, about 6.76, about 18.89, about 21.29, and about 23.16 degrees; (b) peaks at angles 2-theta of about 4.31, about 6.76, about 11.25, about 12.75, about 18.89, about 20.38, about 21.29, about 23.16, about 23.97, and about 24.68 degrees; or (c) peaks at angles 2-theta of about 4.31, about 6.76, about 8.55, about 10.04, about 11.25, about 12.75, about 13.76, about 17.20, about 18.02, about 18.89, about 20.38, about 21.29, about 23.16, about 23.97, about 24.68, about 25.50, about 26.18, about 29.88, and about 33.49 degrees. In some embodiments, the phosphate salt of compound 5 is Form I, characterized as having an XRPD pattern comprising peaks at angles 2-theta of about 4.31 and about 6.76 degrees.

In some embodiments, the fumarate salt of compound 5 is Form II, characterized as having an XRPD pattern comprising: (a) peaks at angles 2-theta of about 8.47, about 10.51, about 13.98, about 23.70, and about 24.43 degrees; (b) peaks at angles 2-theta of about 8.47, about 9.37, about 10.51, about 13.98, about 16.20, about 17.26, about 23.70, about 24.01, about 24.43, and about 25.59 degrees; or (c) peaks at angles 2-theta of about 6.87, about 8.47, about 9.37, about 10.51, about 12.20, about 12.63, about 13.34, about 13.98, about 16.20, about 17.26, about 18.11, about 18.72, about 19.42, about 20.46, about 21.87, about 22.15, about 23.70, about 24.01, about 24.43, and about 25.59 degrees. In some embodiments, the fumarate salt of compound 5 is Form II, characterized as having an XRPD pattern comprising peaks at angles 2-theta of about 8.47 and about 9.37 degrees.

In some embodiments, the 1,5-naphthalenedisulfonate salt of compound 5 is Form III, characterized as having an XRPD pattern comprising: (a) peaks at angles 2-theta of about 15.87 and about 23.85 degrees; (b) peaks at angles 2-theta of about 12.58, about 15.87, about 16.77, about 23.85, and about 31.95 degrees; or (c) peaks at angles 2-theta of about 3.17, about 12.58, about 15.87, about 16.77, about 18.78, about 23.85, about 25.30, about 27.34, about 29.61, about 31.95, about 38.05, and about 38.63 degrees. In some embodiments, the 1,5-naphthalenedisulfonate salt of compound 5 is Form III, characterized as having an XRPD pattern comprising peaks at angles 2-theta of about 12.58 and about 15.87 degrees.

In some embodiments, the mixed solvate of isopropyl alcohol and water of the phosphate salt of compound 5 is Form IV, characterized as having an XRPD pattern comprising peaks at angles 2-theta of about 4.14, about 20.02, about 21.57, about 6.64, and about 25.22 degrees. In some embodiments, the mixed solvate of isopropyl alcohol and water of the phosphate salt of compound 5 is Form IV, characterized as having an XRPD pattern comprising peaks at angles 2-theta of about 4.14 and about 6.64 degrees.

Articles of manufacture comprising a compound of the invention, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms.

### Pharmaceutical Compositions and Formulations

Pharmaceutical compositions of any of the compounds detailed herein, including compound 5 or a salt thereof, are embraced by this invention. Thus, the invention includes pharmaceutical compositions comprising an integrin modulating compound, such as compound 5, or a pharmaceutically acceptable salt thereof and a pharmaceutically acceptable carrier or excipient. In one aspect, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation. In one embodiment, the pharmaceutical composition is a composition for controlled release of any of the compounds detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

In one embodiment, the compounds can be administered in the liquid vehicle ORA-SWEET® from PERRIGO®, Allegan, Michigan, which is a syrup vehicle having ingredients of purified water, glycerin, sorbitol, sodium saccharin, xanthan gum, and flavoring, buffered with citric acid and sodium citrate, preserved with methylparaben (0.03%), potassium sorbate (0.1%), and propylparaben (0.008%); or in a mixture of ORA-SWEET® and water of any proportion, such as a 50:50 mixture of ORA-SWEET® to water. The water used should be a pharmaceutically acceptable grade of water, for example, sterile water.

Any of the compounds described herein can be formulated in a tablet in any dosage form described, for example, a compound as described herein or a pharmaceutically acceptable salt thereof can be formulated as a 10 mg tablet.

Compositions comprising a compound provided herein are also described. In one variation, the composition comprises a compound and a pharmaceutically acceptable carrier or excipient. In another variation, a composition of substantially pure compound is provided. In some embodiments, the composition is for use as a human or veterinary medicament. In some embodiments, the composition is for use in a method described herein. In some embodiments, the composition is for use in the treatment of a disease or disorder described herein.

### Methods of Use

Compounds and compositions of the invention, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In one aspect, provided is method for determining a therapeutic dose of a compound, or a pharmaceutically acceptable salt thereof, for treating a condition mediated by at least one integrin, comprising: (a) administering the compound, or a pharmaceutically acceptable salt thereof, to a subject in need of therapy for the condition, wherein the subject expresses the at least one integrin and the compound, or pharmaceutically acceptable salt thereof, binds to the at least one integrin; (b) measuring the percent occupancy of the compound, or a pharmaceutically acceptable salt thereof, in the at least one integrin; and (c) determining the amount of the compound, or a pharmaceutically acceptable salt thereof, that is effective to achieve a predetermined percent occupancy of the at least one integrin, wherein the amount of compound or pharmaceutically acceptable salt thereof that is required to achieve the predetermined percent occupancy is the therapeutic dose of the compound or a pharmaceutically acceptable salt thereof. In some embodiments, the subject has a tissue involved in the condition that expresses the at least one integrin, and the at least one integrin is in the tissue of the subject, Thus, in some embodiments, provided is method for determining a therapeutic dose of a compound, or a pharmaceutically acceptable salt thereof, for treating a condition mediated by at least one integrin, comprising: (a) administering the compound, or a pharmaceutically acceptable salt thereof, to a subject in need of therapy for the condition, wherein the subject has a tissue involved in the condition that expresses the at least one integrin and the compound, or pharmaceutically acceptable salt thereof, binds to the at least one integrin; (b) measuring the percent occupancy of the compound, or a pharmaceutically acceptable salt thereof, in the at least one integrin in the tissue of the subject; and (c) determining the amount of the compound, or a pharmaceutically acceptable salt thereof, that is effective to achieve a predetermined percent occupancy of the at least one integrin in the tissue of the subject, wherein the amount of compound or pharmaceutically acceptable salt thereof that is required to achieve the predetermined percent occupancy is the therapeutic dose of the compound or a pharmaceutically acceptable salt thereof. In some embodiments, the subject has a tissue involved in the condition that expresses the at least one integrin, and the at least one integrin is in the tissue of the subject.

In some embodiments, (a) and (b) are performed on a first subject and one or more additional subjects, wherein the first and the one or more additional subjects have the same condition, and wherein the first subject is administered a first amount of the compound or a pharmaceutically acceptable salt thereof and the one or more additional subjects are each administered an amount of the compound or a pharmaceutically acceptable salt thereof that is different than the amount given to the first subject.

In some embodiments, the compound, or a pharmaceutically acceptable salt thereof, is administered to the subject in a single dose. In some embodiments, the compound, or a pharmaceutically acceptable salt thereof, is administered to the subject in a two or more doses. In some embodiments, the first and subsequent doses are administered up to two or three weeks apart.

In another aspect, provided is a method for determining the therapeutically effective percent occupancy of an integrin of a compound that binds to the integrin, or a pharmaceutically acceptable salt thereof, the method comprising:
(a) administering the compound, or a pharmaceutically acceptable salt thereof, to a subject having a condition mediated by at least one integrin and the subject expresses the at least one integrin;
(b) determining the percent occupancy of the compound in at least one integrin in the subject;
(c) measuring in the subject one or more parameters associated with the condition before and after administration of the compound, or a pharmaceutically acceptable salt thereof; and
wherein a beneficial effect on one or more parameters associated with the condition measured after administration of the compound, or pharmaceutically acceptable salt thereof, compared to measurement of the same parameter in the subject before administration of the compound, or a pharmaceutically acceptable salt thereof, indicates a therapeutically effective percent occupancy of the integrin. In some embodiments, the subject has a tissue that expresses the at least one integrin. In some embodiments, the method comprises determining the percent occupancy of the compound in at least one integrin in the tissue of the subject. Thus, in some embodiments, provided is a method for determining the therapeutically effective percent occupancy of an integrin of a compound that binds to the integrin, or a pharmaceutically acceptable salt thereof, the method comprising:
(a) administering the compound, or a pharmaceutically acceptable salt thereof, to a subject having a condition mediated by at least one integrin and a tissue that expresses the at least one integrin;
(b) determining the percent occupancy of the compound in at least one integrin in the tissue of the subject;
(c) measuring in the subject one or more parameters associated with the condition before and after administration of the compound, or a pharmaceutically acceptable salt thereof, and
wherein a beneficial effect on one or more parameters associated with the condition measured after administration of the compound, or pharmaceutically acceptable salt thereof, compared to measurement of the same parameter in the subject before administration of the compound, or a pharmaceutically acceptable salt thereof, indicates a therapeutically effective percent occupancy of the integrin.

In another aspect, provided is a method of delaying the onset and/or development of a fibrotic disease in an individual (such as a human) who is at risk for developing a fibrotic disease. It is appreciated that delayed development may encompass prevention in the event the individual does not develop the fibrotic disease. An individual at risk of developing a fibrotic disease in one aspect has or is suspected of having one or more risk factors for developing a fibrotic disease. Risk factors for fibrotic disease may include an individual's age (e.g., middle-age or older adults), the presence of inflammation, having one or more genetic component associated with development of a fibrotic disease, medical history such as treatment with a drug or procedure believed to be associated with an enhanced susceptibility to fibrosis (e.g., radiology) or a medical condition believed to be associated with fibrosis, a history of smoking, the presence of occupational and/or environmental factors such as exposure to pollutants associated with development of a fibrotic disease. In some embodiments, the individual at risk for developing a fibrotic disease is an individual who has or is suspected of having NAFLD, NASH, CKD, scleroderma, Crohn's Disease, NSIP, PSC, PBC, or is an individual who has had or is suspected of having had a myocardial infarction. In some embodiments, the individual at risk for developing a fibrotic disease has or is suspected of having psoriasis.

In some embodiments, the fibrotic disease is fibrosis of a tissue such as the lung (pulmonary fibrosis), the liver, the skin, the heart (cardiac fibrosis), the kidney (renal fibrosis), or the gastrointestinal tract (gastrointestinal fibrosis).

In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC). In some embodiments, the fibrotic disease is pulmonary fibrosis (such as IPF), liver fibrosis, skin fibrosis, psoriasis, scleroderma, cardiac fibrosis, renal fibrosis, gastrointestinal fibrosis, primary sclerosing cholangitis, or biliary fibrosis (such as PBC). In some embodiments, the fibrotic disease is psoriasis.

In some embodiments, the fibrotic disease is a pulmonary fibrosis, e.g., idiopathic pulmonary fibrosis (IPF). In some embodiments, the pulmonary fibrosis is, e.g., interstitial lung disease, radiation-induced pulmonary fibrosis, or systemic sclerosis associated interstitial lung disease.

In some embodiments, the fibrotic disease is a primary sclerosing cholangitis, or biliary fibrosis. In some embodiments, the fibrotic disease is primary biliary cholangitis (also known as primary biliary cirrhosis) or biliary atresia.

In some embodiments, the fibrotic disease is fibrotic nonspecific interstitial pneumonia (NSIP).

In some embodiments, the fibrotic disease is a liver fibrosis, e.g., infectious liver fibrosis (from pathogens such as HCV, HBV or parasites such as schistosomiasis), NASH, alcoholic steatosis induced liver fibrosis, and cirrhosis. In some embodiments, the liver fibrosis is nonalcoholic fatty liver disease (NAFLD). In some embodiments, the liver fibrosis is NASH.

In some embodiments, the fibrotic disease is biliary tract fibrosis.

In some embodiments, the fibrotic disease is renal fibrosis, e.g., diabetic nephrosclerosis, hypertensive nephrosclerosis, focal segmental glomerulosclerosis (“FSGS”), and acute kidney injury from contrast induced nephropathy. In several embodiments, the fibrotic disease is diabetic nephropathy, diabetic kidney disease, or chronic kidney disease.

In some embodiments, the fibrotic disease is characterized by one or more of glomerulonephritis, end-stage kidney disease, hearing loss, changes to the lens of the eye, hematuria, or proteinuria. In some embodiments, the fibrotic disease is Alport syndrome.

In some embodiments, the fibrotic disease is systemic and local sclerosis or scleroderma, keloids and hypertrophic scars, or post surgical adhesions. In some embodiments, the fibrotic disease is scleroderma or systemic sclerosis.

In some embodiments, the fibrotic disease is atherosclerosis or restenosis.

In some embodiments, the fibrotic disease is a gastrointestinal fibrosis, e.g., Crohn's disease.

In some embodiments, the fibrotic disease is cardiac fibrosis, e.g., post myocardial infarction induced fibrosis and inherited cardiomyopathy.

In some embodiments, the fibrotic disease is psoriasis.

In some embodiments, methods may include modulating the activity of at least one integrin in a subject in need thereof. For example, the method may include modulating the activity of α_{V}β₆. The method may include modulating the activity of α_{V}β₁. The method may include modulating the activity of α_{V}β₁ and α_{V}β₆. Modulating the activity of the at least one integrin may include, e.g., inhibiting the at least one integrin. The method may include administering to the subject an amount of the compound or a pharmaceutically acceptable salt thereof effective to modulate the activity of the at least one integrin in the subject, e.g., at least one of α_{V}β₁ and α_{V}β₆. The subject in need of modulating the activity of at least one integrin may have any of the fibrotic disease or conditions described herein. For example, the fibrotic disease or condition may include idiopathic pulmonary fibrosis, interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis, primary biliary cholangitis (also known as primary biliary cirrhosis), biliary atresia, systemic sclerosis associated interstitial lung disease, scleroderma (also known as systemic sclerosis), diabetic nephropathy, diabetic kidney disease, focal segmental glomerulosclerosis, chronic kidney disease, or Crohn's Disease. The fibrotic disease or condition may include psoriasis. The method may include administering to the subject an amount of the compound or a pharmaceutically acceptable salt thereof effective to modulate the activity of the at least one integrin in the subject, e.g., at least one of α_{V}β₁ and α_{V}β₆, the subject being in need of treatment for NASH. The method may include administering to the subject an amount of the compound or a pharmaceutically acceptable salt thereof effective to modulate the activity of the at least one integrin in the subject, e.g., at least one of α_{V}β₁ and α_{V}β₆, the subject being in need of treatment for IPF.

The fibrotic disease may be mediated primarily by α_{V}β₆, for example, the fibrotic disease may include idiopathic pulmonary fibrosis or renal fibrosis. Accordingly, the method may include modulating the activity of α_{V}β₆ to treat conditions primarily mediated by α_{V}β₆ such as IPF. The fibrotic disease may be mediated primarily by α_{V}β₁, for example, the fibrotic disease may include NASH. Accordingly, the method may include modulating the activity of α_{V}β₁ to treat conditions primarily mediated by α_{V}β₁, e.g., NASH. The fibrotic disease may be mediated by α_{V}β₁ and α_{V}β₆, for example, the fibrotic disease may include PSC or biliary atresia. Accordingly, the method may include modulating the activity of α_{V}β₁ and α_{V}β₆ to treat conditions mediated by both α_{V}β₁ and α_{V}β₆.

The compound may be a modulator, e.g., an inhibitor, of α_{V}β₁. The compound may be a modulator, e.g., an inhibitor, of α_{V}β₆. The compound may be a dual modulator, such as a dual inhibitor, e.g., dual selective inhibitor, of α_{V}β₁ and α_{V}β₆.

Modulating or inhibiting the activity of one or both of α_{V}β₁ integrin and α_{V}β₆ integrin, thereby treating a subject with a fibrotic disease, indicates that α_{V}β₁ integrin, α_{V}β₆ integrin, or α_{V}β₁ integrin and α_{V}β₆ integrin are modulated or inhibited to a degree sufficient to treat the fibrotic disease in the subject.

In another aspect, provided herein is a method of treating a subject in need thereof, comprising administering to the subject a therapeutically effective amount of an integrin modulating compound, such as compound 5, or a pharmaceutically acceptable salt thereof, or a dosage form disclosed herein, wherein the subject has at least one tissue in need of therapy and the tissue has at least one elevated level of: αVβ1 integrin activity and/or expression; αVβ6 integrin activity and/or expression; a pSMAD/SMAD value; new collagen formation or accumulation; total collagen; and Type I Collagen gene Col1a1 expression; and wherein the level is elevated compared to a healthy state of the tissue. In some embodiments, the at least one tissue in the subject comprises one or more of: blood, lung tissue, liver tissue, skin tissue, cardiac tissue, kidney tissue, gastrointestinal tissue, gall bladder tissue, and bile duct tissue. In some embodiments, the at least one tissue in the subject comprises one or more of: lung tissue, liver tissue, skin tissue, cardiac tissue, kidney tissue, gastrointestinal tissue, gall bladder tissue, and bile duct tissue. In some embodiments, the tissue has an elevated pSMAD2/SMAD2 value or an elevated pSMAD3/SMAD3 value compared to the healthy state of the tissue.

Methods of determining the values of αVβ1 integrin activity and/or expression; αVβ6 integrin activity and/or expression; a pSMAD/SMAD value; new collagen formation or accumulation; total collagen; and Type I Collagen gene Col1a1 expression are known in the art and exemplary methods are disclosed in the Examples, such as antibody assays of tissue samples, such as a biopsy sample.

In some embodiments, the method selectively reduces αVβ1 integrin activity and/or expression compared to αVβ6 integrin activity and/or expression in the subject. In some embodiments, the method selectively reduces α_{V}β₆ integrin activity and/or expression compared to α_{V} β₁ integrin activity and/or expression in the subject. In some embodiments, the method reduces both α_{V} β₁ integrin and α_{V}β₆ integrin activity and/or expression compared to at least one other α_{V}-containing integrin in the subject. In some embodiments, the activity of αVβ1 integrin in one or more fibroblasts is reduced in the subject. In some embodiments, the activity of αVβ6 integrin in one or more epithelial cells is reduced in the subject.

In another aspect, provided herein is a method of treating a subject in need thereof, comprising administering to the subject a therapeutically effective amount of an integrin modulating compound, such as compound 5, or a pharmaceutically acceptable salt thereof, or a dosage form disclosed herein, wherein the subject has at least one tissue in need of therapy and the tissue has at least one elevated level of: αVβ1 integrin activity and/or expression; αVβ6 integrin activity and/or expression; a pSMAD/SMAD value; new collagen formation or accumulation; total collagen; and Type I Collagen gene Col1a1 expression; and wherein the level is elevated compared to a healthy state of the tissue. In some embodiments, the at least one tissue in the subject comprises one or more of: lung tissue, liver tissue, skin tissue, cardiac tissue, kidney tissue, gastrointestinal tissue, gall bladder tissue, and bile duct tissue. In some embodiments, the tissue has an elevated pSMAD2/SMAD2 value or an elevated pSMAD3/SMAD3 value compared to the healthy state of the tissue.

Methods of determining the values of αVβ1 integrin activity and/or expression; αVβ6 integrin activity and/or expression; a pSMAD/SMAD value; new collagen formation or accumulation; total collagen; and Type I Collagen gene Col1a1 expression are known in the art and exemplary methods are disclosed in the Examples, such as antibody assays of tissue samples, such as a biopsy sample.

In some embodiments, the method selectively reduces αVβ1 integrin activity and/or expression compared to αVβ6 integrin activity and/or expression in the subject. In some embodiments, the method selectively reduces α_{V}β₆ integrin activity and/or expression compared to α_{V} β₁ integrin activity and/or expression in the subject. In some embodiments, the method reduces both α_{V} β₁ integrin and α_{V}β₆ integrin activity and/or expression compared to at least one other α_{V}-containing integrin in the subject. In some embodiments, the activity of αVβ1 integrin in one or more fibroblasts is reduced in the subject. In some embodiments, the activity of αVβ6 integrin in one or more epithelial cells is reduced in the subject.

Also provided herein is a method of characterizing the antifibrotic activity of a small molecule in a subject, comprising: providing a first live cell sample from the subject, the first live cell sample characterized by the presence of at least one integrin capable of activating transforming growth factor β (TGF-β) from latency associated peptide-TGF-β; determining a first pSMAD/SMAD value in the first live cell sample; administering the small molecule to the subject; providing a second live cell sample from the subject, the second live cell sample being drawn from the same tissue in the subject as the first live cell sample; determining a second pSMAD/SMAD value in the second live cell sample; and characterizing the antifibrotic activity of the small molecule in the subject by comparing the second pSMAD/SMAD value to the first pSMAD/SMAD value. In some embodiments, the small molecule is a compound disclosed herein, optionally in a dosage form disclosed herein.

In another aspect, a method for determining a therapeutic dose for a condition mediated by at least one integrin is provided, the method comprising: providing a subject in need of therapy, the subject comprising: the condition mediated by the at least one integrin, and a tissue involved in the condition that expresses the at least one integrin; modulating the at least one integrin in the tissue in the subject effective to treat the condition, comprising administering to the subject at least one compound that binds to a receptor of the at least one integrin, the at least one compound being administered to the subject in an amount effective to achieve a percent occupancy of the receptor of the at least one integrin in the tissue of the subject of at least about 50%.

In another aspect, a method for therapy for a condition mediated by at least one integrin is provided, the method comprising: providing a subject in need of therapy, the subject comprising: the condition mediated by the at least one integrin, and a tissue involved in the condition that expresses the at least one integrin; and modulating the at least one integrin in the tissue in the subject effective to treat the condition, comprising administering to the subject at least one compound that binds to a receptor of the at least one integrin, the at least one compound being administered to the subject in an amount effective to achieve a percent occupancy of the receptor of the at least one integrin in the tissue of the subject of at least about 50%. Thus, provided herein is a method of treating a condition mediated by at least one integrin in a subject, comprising: administering the subject at least one compound, or pharmaceutically acceptable salt thereof, that binds to a receptor of the at least one integrin, the at least one compound being administered to the subject in an amount effective to achieve a percent occupancy of the receptor of the at least one integrin in the tissue of the subject of at least about 50%, wherein the subject has a condition mediated by the at least one integrin. In some embodiments, the subject, such as a human, is in need of therapy, such as a subject having IPF or PSC. In some embodiments the subject has a tissue, such as lung tissue, that expresses the at least one integrin. In some embodiments, the at least one integrin is α_{V}β₁ integrin or α_{V}β₆ integrin. In some embodiments, the at least one integrin is α_{V}β₁ integrin and α_{V}β₆ integrin. In some embodiments, the % occupancy is measured at about 4 hours following administration of the compound. In some embodiments, percent occupancy is measured using data obtained from a PET/CT scan of the subject after administration of the compound, or a pharmaceutically acceptable salt thereof.

In another aspect, a method for therapy for a condition mediated by at least one integrin is provided, the method comprising: providing a subject in need of therapy, the subject comprising: the condition mediated by the at least one integrin, and a tissue involved in the condition that expresses the at least one integrin; and modulating the at least one integrin in the tissue in the subject effective to treat the condition, comprising administering to the subject at least one compound that binds to a receptor of the at least one integrin, the at least one compound being administered to the subject in an amount effective to achieve a percent occupancy of the receptor of the at least one integrin in the tissue of the subject of at least about 50% over a period of at least about 24 hours.

In some embodiments, percent occupancy corresponds to an anti-fibrotic effect. In some embodiments, the antifibrotic effect comprises a reduction in SMAD phosphorylation. In some embodiments, the antifibrotic effect is determined by histology or quantitative fibrosis imaging.

In some embodiments, the therapeutic dose achieves between about 50% and about 99% receptor occupancy of the at least one integrin. In some embodiments, the therapeutic dose achieves between about 50% and about 90% receptor occupancy of the at least one integrin. In some embodiments, the therapeutic dose achieves between about 60% and about 99% receptor occupancy of the at least one integrin. In some embodiments, the therapeutic dose achieves between about 60% and about 90% receptor occupancy of the at least one integrin. In some embodiments, the therapeutic dose achieves between about 70% and about 99% receptor occupancy of the at least one integrin. In some embodiments, the therapeutic dose achieves between about 70% and about 90% receptor occupancy of the at least one integrin. In some embodiments, the therapeutic dose achieves between about 80% and about 99% receptor occupancy of the at least one integrin. In some embodiments, the therapeutic dose achieves between about 80% and about 90% receptor occupancy of the at least one integrin.

In some embodiments, the therapeutic dose achieves about 50% or greater receptor occupancy of the at least one integrin. In some embodiments, the therapeutic dose achieves 50% or greater receptor occupancy of the at least one integrin. In some embodiments, the therapeutic dose achieves about 60% or greater receptor occupancy of the at least one integrin. In some embodiments, the therapeutic dose achieves 60% or greater receptor occupancy of the at least one integrin. In some embodiments, the therapeutic dose achieves about 70% or greater receptor occupancy of the at least one integrin. In some embodiments, the therapeutic dose achieves 70% or greater receptor occupancy of the at least one integrin. In some embodiments, the therapeutic dose achieves about 80% or greater receptor occupancy of the at least one integrin. In some embodiments, the therapeutic dose achieves 80% or greater receptor occupancy of the at least one integrin. In some embodiments, the therapeutic dose achieves about 90% or greater receptor occupancy of the at least one integrin. In some embodiments, the therapeutic dose achieves 90% or greater receptor occupancy of the at least one integrin. In some embodiments, the therapeutic dose achieves about 95% or greater receptor occupancy of the at least one integrin. In some embodiments, the therapeutic dose achieves 95% or greater receptor occupancy of the at least one integrin. In some embodiments, the therapeutic dose achieves about 98% or greater receptor occupancy of the at least one integrin. In some embodiments, the therapeutic dose achieves 98% or greater receptor occupancy of the at least one integrin. In some embodiments, the therapeutic dose achieves about 99% or greater receptor occupancy of the at least one integrin. In some embodiments, the therapeutic dose achieves 99% or greater receptor occupancy of the at least one integrin.

In some embodiments, the receptor occupancy of the at least one integrin is measured at Cₘᵢₙ. In some embodiments, the receptor occupancy of the at least one integrin is measured at Cₘₐₓ. In some embodiments, at least about 50% receptor occupancy is achieved with a plasma concentration of at least about 5 nM. In some embodiments, at least about 50% receptor occupancy is achieved with a plasma concentration of at least about 10 nM. In some embodiments, at least about 50% receptor occupancy is achieved with a plasma concentration of about 100 nM. In some embodiments, at least about 60% receptor occupancy is achieved with a plasma concentration of at least about 10 nM. In some embodiments, at least about 70% receptor occupancy is achieved with a plasma concentration of at least about 10 nM. In some embodiments, at least about 90% receptor occupancy is achieved with a plasma concentration of about 50 nM. In some embodiments, at least about 70% receptor occupancy is achieved with a plasma concentration of about 100 nM. In some embodiments, at least about 75% receptor occupancy is achieved with a plasma concentration of about 100 nM. In some embodiments, at least about 80% receptor occupancy is achieved with a plasma concentration of about 100 nM.

In some embodiments, at least about 50% receptor occupancy is achieved with a dose of about 60 mg of the compound. In some embodiments, at least about 60% receptor occupancy is achieved with a dose of about 120 mg of the compound. In some embodiments, at least about 70% receptor occupancy is achieved with a dose of about 240 mg of the compound. In some embodiments, at least about 95% receptor occupancy is achieved with a dose of about 240 mg of the compound. In some embodiments, at least about 80% receptor occupancy is achieved with a dose of about 320 mg of the compound. In some embodiments, at least about 90% receptor occupancy is achieved with a dose of about 320 mg of the compound.

In some embodiments, the at least one compound that binds to a receptor of the at least one integrin is administered once daily. In some embodiments, the at least one compound that binds to a receptor of the at least one integrin is administered twice daily. In some embodiments, the at least one compound that binds to a receptor of the at least one integrin is administered three times daily. In some embodiments, the at least one compound that binds to a receptor of the at least one integrin is administered four times daily. In some embodiments, the at least one compound that binds to a receptor of the at least one integrin is administered once daily for 2, 3, 4, or 6 weeks. In some embodiments, the at least one compound that binds to a receptor of the at least one integrin is administered twice daily for 2, 3, 4, or 6 weeks. In some embodiments, the at least one compound that binds to a receptor of the at least one integrin is administered three times daily for 2, 3, 4, or 6 weeks.

In some embodiments, the receptor occupancy of the at least one integrin is achieved at least at about the specified percentage (e.g., 50%, 60%, 70%, 80%, 90%, 95%, 98%, or 99%) over a period of at least about 24 hours. By way of non-limiting example, in some embodiments, the receptor occupancy of the at least one integrin is about 50% or greater for at least about 4 hours. In some embodiments, the receptor occupancy of the at least one integrin is about 50% or greater for at least about 6 hours. In some embodiments, the receptor occupancy of the at least one integrin is about 50% or greater for at least about 8 hours. In some embodiments, the receptor occupancy of the at least one integrin is about 50% or greater for at least about 12 hours. In some embodiments, the receptor occupancy of the at least one integrin is about 50% or greater for at least about 24 hours.

In some embodiments, the percent occupancy of the receptor of the at least one integrin in the tissue of the subject of at least about 50% over a period of at least about one of, or a range between about any two of: 2, 3, 4, 6, 8, 12, 24, 48, 72, 168, 336, or 672 hours. In some embodiments, the receptor occupancy of the at least one integrin is about 50% or greater for at least about 4 hours to about 7 days. In some embodiments, the receptor occupancy of the at least one integrin is about 50% or greater for at least about 6 hours to about 7 days. In some embodiments, the receptor occupancy of the at least one integrin is about 50% or greater for at least about 8 hours to about 7 days. In some embodiments, the receptor occupancy of the at least one integrin is about 50% or greater for at least about 12 hours to about 7 days. In some embodiments, the receptor occupancy of the at least one integrin is about 50% or greater for at least about 24 hours to about 7 days.

In some embodiments, the receptor occupancy of the at least one integrin is about 50% or greater for at least about 4 hours to about 24 hours. In some embodiments, the receptor occupancy of the at least one integrin is about 50% or greater for at least about 6 hours to about 24 hours. In some embodiments, the receptor occupancy of the at least one integrin is about 50% or greater for at least about 8 hours to about 24 hours. In some embodiments, the receptor occupancy of the at least one integrin is about 50% or greater for at least about 12 hours to about 24 hours. In some embodiments, the receptor occupancy of the at least one integrin is about 50% or greater for at least about 24 hours.

In some embodiments, the receptor occupancy of the at least one integrin is about 50% or greater for at least about 24 hours to about 48 hours. In some embodiments, the receptor occupancy of the at least one integrin is about 50% or greater for at least about 24 hours to about 72 hours. In some embodiments, the receptor occupancy of the at least one integrin is about 50% or greater for at least about 24 hours to about 96 hours. In some embodiments, the receptor occupancy of the at least one integrin is about 50% or greater for at least about 24 hours to about 7 days. In some embodiments, the receptor occupancy of the at least one integrin is about 50% or greater for at least about 24 hours to about 14 days. In some embodiments, the receptor occupancy of the at least one integrin is about 50% or greater for at least about 24 hours to about one month

In some embodiments, the receptor occupancy of the at least one integrin is achieved at least at about the specified percentage (e.g., 50%, 60%, 70%, 80%, 90%, 95%, 98%, or 99%) for at least about 8, 12, 18, 24 hours. By way of non-limiting example, in some embodiments, the receptor occupancy of the at least one integrin is about 50% or greater for at least about 8 hours. In some embodiments, the receptor occupancy of the at least one integrin is about 50% or greater for at least 8 hours. In some embodiments, the receptor occupancy of the at least one integrin is about 50% or greater for at least about 12 hours. In some embodiments, the receptor occupancy of the at least one integrin is about 50% or greater for at least 12 hours. In some embodiments, the receptor occupancy of the at least one integrin is about 50% or greater for at least about 18 hours. In some embodiments, the receptor occupancy of the at least one integrin is about 50% or greater for at least 18 hours. In some embodiments, the receptor occupancy of the at least one integrin is about 50% or greater for at least about 24 hours. In some embodiments, the receptor occupancy of the at least one integrin is about 50% or greater for at least 24 hours.

In some embodiments, the receptor occupancy of the at least one integrin is achieved at least at about the specified percentage (e.g., 50%, 60%, 70%, 80%, 90%, 95%, 98%, or 99%) for at least about 8, 12, 18 or 24 hours by dosing the compound once daily or twice daily. By way of non-limiting example, in some embodiments, the receptor occupancy of the at least one integrin is about 50% or greater for at least about 8 hours when the compound is dosed once daily. In some embodiments, the receptor occupancy of the at least one integrin is about 50% or greater for at least about 8 hours when the compound is dosed twice daily. In some embodiments, the receptor occupancy of the at least one integrin is about 50% or greater for at least 8 hours when the compound is dosed once daily. In some embodiments, the receptor occupancy of the at least one integrin is about 50% or greater for at least 8 hours when the compound is dosed twice daily. In some embodiments, the receptor occupancy of the at least one integrin is about 50% or greater for at least about 12 hours when the compound is dosed once daily. In some embodiments, the receptor occupancy of the at least one integrin is about 50% or greater for at least about 12 hours when the compound is dosed twice daily. In some embodiments, the receptor occupancy of the at least one integrin is about 50% or greater for at least 12 hours when the compound is dosed once daily. In some embodiments, the receptor occupancy of the at least one integrin is about 50% or greater for at least 12 hours when the compound is dosed twice daily. In some embodiments, the receptor occupancy of the at least one integrin is about 50% or greater for at least about 18 hours when the compound is dosed once daily. In some embodiments, the receptor occupancy of the at least one integrin is about 50% or greater for at least about 18 hours when the compound is dosed twice daily. In some embodiments, the receptor occupancy of the at least one integrin is about 50% or greater for at least 18 hours when the compound is dosed once daily. In some embodiments, the receptor occupancy of the at least one integrin is about 50% or greater for at least 18 hours when the compound is dosed twice daily. In some embodiments, the receptor occupancy of the at least one integrin is about 50% or greater for at least about 24 hours when the compound is dosed once daily. In some embodiments, the receptor occupancy of the at least one integrin is about 50% or greater for at least about 24 hours when the compound is dosed twice daily. In some embodiments, the receptor occupancy of the at least one integrin is about 50% or greater for at least 24 hours when the compound is dosed once daily. In some embodiments, the receptor occupancy of the at least one integrin is about 50% or greater for at least 24 hours when the compound is dosed twice daily.

In some embodiments, receptor occupancy is measured by positron emission tomography. In some embodiments, percent occupancy is measured using data obtained from a PET/CT scan of the subject after administration of the compound, or a pharmaceutically acceptable salt thereof. In some embodiments, receptor occupancy is determined by competitive binding at the integrin with a radiolabeled competitive binding agent. In some embodiments, the compound displaces a radiolabeled competitive binding agent. In some embodiments, the compound displaces a knottin radiotracer.

Elevated levels of integrins, particularly β₆, are associated with progressive fibrosis (Saini, G., et al., Eur Respir J 2015; 46:486-494; Bowman, W. S., et al., Lancet Respir Med 2022. Published Online Jan. 18, 2022, https://doi.org/10.1016/S2213-2600(21)00503-8, each hereby incorporated by reference in its entirety). In some embodiments, the subject has a disorder as measured by elevated levels of at least one integrin. In some embodiments, the subject has a disorder as measured by elevated levels of at least one integrin comprising a β₆ subunit. In some embodiments, subjects with more advanced disease states have elevated levels of at least one integrin. In some embodiments, subjects with more advanced disease states have elevated levels of at least one integrin comprising a β₆ subunit. In some embodiments, the disorder is IPF.

Interstitial lung disease (ILD) is a syndrome with many different diseases in the class including, e.g., IPF. IPF is the more progressive disease with severe mortality. Each ILD may progress and dramatically reduce survival, but this is more common with IPF.

Diagnostic criteria for IPF are disclosed in Am J Respir Crit Care Med Vol 205, Iss 9, pp e18-e47, May 1, 2022, herein incorporated by reference in its entirety. For patients suspected of having IPF, diagnosis comprises obtaining high-resolution computed tomography (HRCT). Patients with a radiological pattern of probable usual interstitial pneumonia (UIP) can receive a diagnosis of IPF after multidisciplinary discussion without confirmation by lung biopsy. BAL with or without transbronchial lung cryobiopsy (TBLC) and/or surgical lung biopsy (SLB) may also be used for diagnosis. Diagnostic criteria for PSC are disclosed in Gastroenterology, 2021; 161:1764-1775, herein incorporated by reference in its entirety. PSC can be diagnosed via magnetic resonance cholangiopancreatography (MRCP) and/or endoscopic retrograde cholangiography (ERC) and may include other criteria including, for example, elevated serum markers of cholestasis, concurrent clinical or histologic features of inflammatory bowel disease (IBD), and/or histologic features compatible with PSC. Thus, in some embodiments, the subject has interstitial lung disease, pulmonary fibrosis, IPF, or PSC. In some embodiments, the subject has interstitial lung disease, pulmonary fibrosis, or IPF. In some embodiments, the subject has pulmonary fibrosis, or IPF. In some embodiments, the subject has IPF. In some embodiments, the subject has PSC. In some embodiments, the subject has a disorder as measured by CT, such as HRCT, and optionally a biopsy such as transbronchial lung cryobiopsy or surgical lung biopsy. In some embodiments, the subject has a disorder as measured by MRCP, ERC, and optionally elevated serum markers of cholestasis, concurrent clinical or histologic features of IBD, and/or histologic features compatible with PSC.

In some embodiments, the at least one compound or pharmaceutically acceptable salt thereof being administered to the subject in an amount effective to achieve a percent occupancy of the compound in the tissue of the subject of about one of, or a range between about any two of: 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, the at least one compound or pharmaceutically acceptable salt thereof being administered to the subject in an amount effective to achieve a percent occupancy of the compound in the tissue of the subject of about one of, or a range between about any two of: 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, the at least one compound being administered to the subject in an amount effective to achieve a percent occupancy of the receptor in the tissue of the subject of about one of, or a range between about any two of: 50%, 55%, 60%, 65%, 70%, 75%, 80%, 85%, 90%, 95%, 96%, 97%, 98%, 99%, or 100%. In some embodiments, compound 5, or a pharmaceutically acceptable salt thereof, is administered to the subject in an amount effective to achieve a percent occupancy of the receptor in the tissue of the subject of about one of 50%, 55%, 60%, 65%, 70%, 75%, 80%, 85%, 90%, 95%, 96%, 97%, 98%, 99%, or 100%. In some embodiments, the at least one compound is orally administered to the subject. In some embodiments, the percent occupancy of the receptor in the tissue of the subject is achieved using a single dose of the compound. In some embodiments, the compound inhibits the at least one integrin in the subject effective to treat the condition. In some embodiments, the tissue has at least one elevated level selected from the group consisting of: activity and/or expression of the at least one integrin; a pSMAD/SMAD value; new collagen formation or accumulation; total collagen; and Type I Collagen gene Col1a1 expression; wherein the level is elevated compared to a healthy state of the tissue. In some embodiments, the elevated pSMAD/SMAD value is at least one of an elevated pSMAD2/SMAD2 value or an elevated pSMAD3/SMAD3 value. In some embodiments, the tissue is selected from at least one member of the group consisting of: lung tissue, liver tissue, skin tissue, cardiac tissue, kidney tissue, gastrointestinal tissue, gall bladder tissue, and bile duct tissue.

In some embodiments, the at least one integrin comprises an α_{V} subunit. In some embodiments, the at least one integrin comprises a β₁ or a β₆ subunit. In some embodiments, the at least one integrin comprises at least one of: α_{V}β₁ integrin and α_{V}β₆ integrin. In some embodiments, the at least one integrin comprises α_{V}β₁ integrin and α_{V}β₆ integrin.

In some embodiments, the condition mediated by the at least one integrin is a fibrotic disease selected from the group consisting of: idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, scleroderma, diabetic nephropathy, diabetic kidney disease, focal segmental glomerulosclerosis, chronic kidney disease, and Crohn's Disease. In some embodiments, the condition mediated by the at least one integrin is selected from the group consisting of: a fibrotic disease and psoriasis.

In some embodiments, the compound inhibits the activity of one or both of α_{V}β₁ integrin and α_{V}β₆ integrin in the subject, thereby treating the fibrotic disease in the subject.

In some embodiments, the condition mediated by the at least one integrin is NASH, and the compound inhibits the activity of at least α_{V}β₁ integrin in the subject effective to treat the subject for NASH. In some embodiments, the condition mediated by the at least one integrin is IPF, and the compound inhibits the activity of at least α_{V}β₆ integrin in the subject effective to treat the subject for IPF. In some embodiments, the compound inhibits the activity of α_{V}β₁ integrin and α_{V}β₆ integrin in the subject effective to treat the subject for IPF.

In some embodiments, the condition mediated by the at least one integrin is PSC, and the compound inhibits the activity of at least one of α_{V}β₆ integrin and α_{V}β₁ integrin in in the subject effective to treat the subject for PSC. In some embodiments, the compound inhibits the activity of α_{V}β₁ integrin and α_{V}β₆ integrin in the subject effective to treat the subject for PSC.

In some embodiments, the condition mediated by the at least one integrin is psoriasis. In some embodiments, the condition mediated by the at least one integrin is psoriasis, and the compound inhibits the activity of one or both of α_{V}β₁ integrin and α_{V}β₆ integrin in the subject effective to treat the subject for psoriasis.

In some embodiments, the method selectively reduces α_{V}β₁ integrin activity and/or expression in the tissue compared to at least one other α_{V}-containing integrin in the subject. In some embodiments, the method selectively reduces α_{V}β₁ integrin activity and/or expression in the tissue compared to α_{V}β₆ integrin in the subject. In some embodiments, the method selectively reduces α_{V}β₆ integrin activity and/or expression in the tissue compared to at least one other α_{V}-containing integrin in the subject. In some embodiments, the method selectively reduces α_{V}β₆ integrin activity and/or expression in the tissue compared to α_{V}β₁ integrin in the subject. In some embodiments, the method selectively reduces α_{V}β₆ integrin or α_{V}β₁ integrin activity and/or expression in the tissue compared to at least one other α_{V}-containing integrin in the subject. In some embodiments, the method selectively reduces α_{V}β₆ integrin and α_{V}β₁ integrin activity and/or expression in the tissue compared to at least one other α_{V}-containing integrin in the subject. In some embodiments, the tissue comprises one or more fibroblasts, and the method inhibits α_{V}β₁ integrin in the one or more fibroblasts. In some embodiments, the tissue comprises one or more epithelial cells, and the method inhibits α_{V}β₆ integrin in the one or more epithelial cells.

In some embodiments, a dose of the at least one compound administered to the subject is an amount in milligrams selected from about one of, or at least about one of: 10, 15, 20, 30, 40, 50, 60, 75, 80, 100, 120, 160, 240, 320, 400, 480, 560, 640, 720, 800, 880, 960, or 1040, or a range between any two of the preceding values. In some embodiments, a dose of the at least one compound administered to the subject is an amount in milligrams selected from about one of, or at least about one of: 60,120,240, and 320, or a range between any two of the preceding values. In some embodiments, the compound is administered to the subject in an amount effective to produce an unbound plasma concentration in nM of the compound in the subject selected from about one of, or at least about one of: 1, 10, 15, 20, 25, 30, 40, 50, 60, 75, 80, 90, 100, 125, 150, 200, 250, 500, 750, 1000, or 1250, or a range between any two of the preceding values. In some embodiments, the compound is administered to the subject in an amount effective to produce an unbound plasma concentration in nM of the compound in the subject selected from about one of, or at least about one of: 10, 15, 20, 25, 30, 40, 50, 60, 75, 80, 90, 100, or 125, or a range between any two of the preceding values.

In some embodiments, the compound is administered to the subject in a dosage form configured for daily administration, the dosage form comprising a pharmaceutically acceptable carrier or excipient and a unit dose of the compound, or a salt thereof. In some embodiments, the dosage form comprises about 1, 2.5, 5, 7.5, 10, 15, 20, 25, 30, 35, 40, 50, 75, 80, 85, 90, 95, 100, 105, 110, 115, 120, or 125 mg of the compound, or a range between any two of the preceding values. In some embodiments, the dosage form comprises the compound in mg of about one of: 1, 2.5, 5, 7.5, 10, 15, 20, 25, 30, 35, 40, 50, 60, 75, 80, 85, 90, 95, 100, 105, 110, 115, 120, 125, 150, 175, 200, 225, or 250, or a range between any two of the preceding amounts. In some embodiments, dosage form comprises the compound in mg of about one of: 10, 15, 20, 30, 40, 50, 75, 80, 100, 120, 160, 240, or 320, or a range between any two of the preceding values. In some embodiments, the dosage form comprises the compound in mg of about one of: 320, 400, 480, 560, 640, 720, 800, 880, 960, or 1040, or a range between any two of the preceding values.

In some embodiments, the dosage form comprises the compound in in an amount effective on administration to an individual to produce a Cₘₐₓ in plasma of the individual in ng/mL of at least about one of 700, 750, 800, 850, 900, 950, 1000, 1050, 1100, 1150, 1200, 1250, 1300, 1350, 1400, 1450, or 1500, or a range between any two of the preceding concentrations. In some embodiments, the dosage form comprises the compound in an amount effective on administration to an individual to produce a Cₘₐₓ in plasma of the individual in ng/mL of at least about one of: 1500, 1600, 1700, 1800, 1900, 2000, 2100, 2200, 2300, 2400, or 2500, or a range between any two of the preceding concentrations. In some embodiments, the dosage form comprises the compound in an amount effective on administration to an individual to produce a Cₘₐₓ in ng/mL in plasma of the individual, the Cₘₐₓ corresponding to a plasma-adjusted concentration effective to inhibit a percentage of α_{V}β₆ or α_{V}β₁ in the individual of at least about one of 50, 55, 60, 65, 70, 75, 80, 85, 90, 95, or 100, or a range between any two of the preceding percentages.

In some embodiments, dosage form is configured for daily administration, the method comprising daily administration of the dosage form to the subject. In some embodiments, the dosage form is configured for daily administration, the method comprising administration of the dosage form to the subject one, two, three, or four times daily.

In some embodiments, each live cell sample is a plurality of cells derived from a tissue of the subject, or a plurality of macrophages associated with the tissue of the subject. In some embodiments, the tissue comprises one of: lung tissue, liver tissue, skin tissue, cardiac tissue, kidney tissue, gastrointestinal tissue, gall bladder tissue, and bile duct tissue. In some embodiments, each live cell sample comprises a plurality of alveolar macrophages derived from a bronchoalveolar lavage fluid of the subject.

In some embodiments, the method further comprises conducting a bronchoalveolar lavage on a lung of the subject effective to produce a bronchoalveolar lavage fluid that comprises the plurality of macrophages as a plurality of alveolar macrophages.

In some embodiments, the subject has a fibrotic disease selected from the group consisting of: idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, scleroderma, diabetic nephropathy, diabetic kidney disease, focal segmental glomerulosclerosis, chronic kidney disease, and Crohn's Disease. In some embodiments, the subject has the fibrotic disease psoriasis.

In some embodiments, the at least one integrin comprises α_{V}. In some embodiments, the at least one integrin comprises α_{V}β₁. In some embodiments, the at least one integrin comprises α_{V}β₆.

In some embodiments, determining the first pSMAD/SMAD value in the at least one live cell comprises determining a pSMAD2/SMAD2 value or a pSMAD3/SMAD3 value; and determining the second pSMAD/SMAD value in the at least one live cell after contacting the at least one live cell with the small molecule comprises determining a pSMAD2/SMAD2 value or a pSMAD3/SMAD3 value.

Also provided herein is a method of treating a fibrotic disease in a subject in need thereof, comprising: providing a first live cell sample from the subject, the first live cell sample having at least one integrin capable of activating transforming growth factor β (TGF-β) from latency associated peptide-TGF-β; determining a first pSMAD/SMAD value in the first live cell sample; administering a small molecule to the subject; providing a second live cell sample from the subject, the second live cell sample being drawn from the same tissue in the subject as the first live cell sample; determining a second pSMAD/SMAD value in the second live cell sample; comparing the second pSMAD/SMAD value to the first pSMAD/SMAD value; and administering the small molecule to the subject if the second pSMAD/SMAD value is lower than the first pSMAD/SMAD value. In some embodiments, the small molecule is a compound disclosed herein or a salt thereof, optionally in a dosage form disclosed herein. In some embodiments, the first live cell sample is obtained from the subject prior to treatment with a small molecule.

In some embodiments, each live cell sample is a plurality of cells derived from a tissue of the subject, or a plurality of macrophages associated with the tissue of the subject. In some embodiments, the tissue comprises one of: lung tissue, liver tissue, skin tissue, cardiac tissue, kidney tissue, gastrointestinal tissue, gall bladder tissue, and bile duct tissue. In some embodiments, each live cell sample comprises a plurality of alveolar macrophages derived from a bronchoalveolar lavage fluid of the subject. In some embodiments, the method further comprising conducting a bronchoalveolar lavage on a lung of the subject effective to produce a bronchoalveolar lavage fluid that comprises the plurality of macrophages as a plurality of alveolar macrophages.

In some embodiments, the subject is characterized by having a fibrotic disease selected from the group consisting of: idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, scleroderma, diabetic nephropathy, diabetic kidney disease, focal segmental glomerulosclerosis, chronic kidney disease, and Crohn's Disease. In some embodiments, the subject is characterized by having psoriasis.

In some embodiments, the at least one integrin comprises α_{V}. In some embodiments, the at least one integrin comprises α_{V}β₁. In some embodiments, the at least one integrin comprises a_{V}β₆.

In some embodiments, determining the first pSMAD/SMAD value in the first live cell sample comprises determining a pSMAD2/SMAD2 value or a pSMAD3/SMAD3 value; and determining the second pSMAD/SMAD value in the at least one live cell after contacting the first live cell sample with the small molecule comprises determining a pSMAD2/SMAD2 value or a pSMAD3/SMAD3 value.

In another aspect, provided is a method of inhibiting αvβ6 integrin in an individual comprising administering an integrin modulating compound, such as compound 5, or a pharmaceutically acceptable salt thereof.

Also provided is a method of inhibiting TGFβ activation in a cell comprising administering to the cell an integrin modulating compound, such as compound 5, or a pharmaceutically acceptable salt thereof.

Also provided is a method of inhibiting αvβ6 integrin in an individual in need thereof, comprising administering to the individual an integrin modulating compound, such as compound 5, or a pharmaceutically acceptable salt thereof.

In some embodiments, the method of inhibition is for an individual in need thereof, such as an individual who has or is suspected of having a fibrotic disease, and wherein the method comprises administering to the individual an integrin modulating compound, such as compound 5, or a pharmaceutically acceptable salt thereof.

In any of the described methods, in one aspect the individual is a human, such as a human in need of the method. The individual may be a human who has been diagnosed with or is suspected of having a fibrotic disease. The individual may be a human who does not have detectable disease but who has one or more risk factors for developing a fibrotic disease.

Also provided herein are dosage forms configured for daily administration, comprising a pharmaceutically acceptable carrier or excipient; and a unit dose of an integrin modulating compound, such as compound 5, or a pharmaceutically acceptable salt thereof.

A unit dose, such as a unit dose for daily administration, can comprise about 1, 2.5, 5, 7.5, 10, 15, 20, 25, 30, 35, 40, 50, 75, 80, 85, 90, 95, 100, 105, 110, 115, 120, or 125 mg of the compound, or a range between any two of the preceding values, such as about 1-125, 1-5, 2.5-7.5, 5-15, 10-15, 10-20, 10-25, 10-30, 10-35, 10-40, 10-50, 10-75, 15-20, 15-25, 15-30, 15-35, 15-40, 15-50, 15-75, 20-25, 20-30, 20-35, 20-40, 20-50, 20-75, 25-30, 25-35, 25-40, 25-50, 25-75, 30-35, 30-40, 30-50, 30-75, 35-40, 35-50, 35-75, 40-50, 40-75, 50-75, 50-100, 60-85, 70-90, 70-100, 80-125, 90-125, or 100-125 mg.

A unit dose, such as a unit dose for daily administration, can comprise about 1, 2.5, 5, 7.5, 10, 15, 20, 25, 30, 35, 40, 50, 75, 80, 85, 90, 95, 100, 105, 110, 115, 120, 125, 150, 175, 200, 225, or 250 mg of the compound, or a range between any two of the preceding values, such as about 1-125, 1-250, 1-5, 2.5-7.5, 5-15, 10-15, 10-20, 10-25, 10-30, 10-35, 10-40, 10-50, 10-75, 15-20, 15-25, 15-30, 15-35, 15-40, 15-50, 15-75, 20-25, 20-30, 20-35, 20-40, 20-50, 20-75, 25-30, 25-35, 25-40, 25-50, 25-75, 30-35, 30-40, 30-50, 30-75, 35-40, 35-50, 35-75, 40-50, 40-75, 50-75, 50-100, 50-150, 50-250, 60-85, 70-90, 70-100, 80-125, 90-125, 100-125, 100-150, 100-200, 125-175, 100-225, 100-250, and 150-250 mg. For example, the unit dose may be 10 mg. The unit dose may be 15 mg. The unit dose may be 20 mg. The unit dose may be 30 mg. The unit dose may be 40 mg. The unit dose may be 50 mg. The unit dose may be 60 mg. The unit dose may be 70 mg. The unit dose may be 75 mg. The unit dose may be 80 mg. The unit dose may be 90 mg. The unit dose may be 100 mg. The unit dose may be 110 mg. The unit dose may be 120 mg. The unit dose may be 125 mg. The unit dose may be 150 mg. The unit dose may be 175 mg. The unit dose may be 200 mg. The unit dose may be 225 mg. The unit dose may be 250 mg.

A unit dose, such as a unit dose for daily administration, can comprise the compound in an amount effective on administration to an individual to produce a Cₘₐₓ in plasma of the individual in ng/mL of at least about, or greater than about, one of: 700, 750, 800, 850, 900, 950, 1000, 1050, 1100, 1150, 1200, 1250, 1300, 1350, 1400, 1450, or 1500; or a range between any two of the preceding concentrations, such as 700-1500, 700-900, 800-1300, 750-950, 800-1000, 850-950, 850-1050, 900-1400,900-1300,900-1200,900-1100,950-1050,950-1400,950-1150, 1000-1400, 1000-1300, 1000-1200, and the like. For example, Cₘₐₓ can be about 700 ng/mL or greater. Cₘₐₓ can be about 750 ng/mL or greater. Cₘₐₓ can be about 800 ng/mL or greater. Cₘₐₓ can be about about 850 ng/mL or greater. Cₘₐₓ can be 900 ng/mL or greater. Cₘₐₓ can be about 950 ng/mL or greater. Cₘₐₓ can be about 1000 ng/mL or greater. Cₘₐₓ can be about 1050 ng/mL or greater. Cₘₐₓ can be about 1100 ng/mL or greater. Cₘₐₓ can be about 1200 ng/mL or greater. Cₘₐₓ can be about 1300 ng/mL or greater. Cₘₐₓ can be about 1400 ng/mL or greater. Cₘₐₓ can be about 1500 ng/mL or greater.

A unit dose, such as a unit dose for daily administration, can comprise the compound in an amount effective on administration to an individual to produce a Cₘₐₓ in ng/mL in plasma of the individual, the Cₘₐₓ corresponding to a plasma-adjusted concentration effective to inhibit a percentage of α_{V}36 or α_{V}β₁ in the individual of at least about one of 50, 55, 60, 65, 70, 75, 80, 85, 90, 95, or 100, or a range between any two of the preceding percentages, for example, 50-100, 60-90, 70-90, 75-95, and the like. In some embodiments, the compound may be a dual α_{V}β₆ and α_{V}β₁ inhibitor, and the Cₘₐₓ can correspond to a plasma-adjusted concentration effective to inhibit a percentage of each of α_{V}β₆ and α_{V}β₁ in the individual, each percentage independently selected from the preceding percentages, or a range between any two of the preceding percentages. For example, the plasma-adjusted concentration can be effective to inhibit α_{V}β₆ by at least about 50%. The plasma-adjusted concentration can be effective to inhibit α_{V}β₆ by at least about 60%. The plasma-adjusted concentration can be effective to inhibit α_{V}β₆ by at least about 70%. The plasma-adjusted concentration can be effective to inhibit α_{V}β₆ by at least about 80%. The plasma-adjusted concentration can be effective to inhibit α_{V}β₆ by at least about 90%. Further, for example, the plasma-adjusted concentration can be effective to inhibit α_{V}β₁ by at least about 50%. The plasma-adjusted concentration can be effective to inhibit α_{V}β₁ by at least about 60%. The plasma-adjusted concentration can be effective to inhibit α_{V}β₁ by at least about 70%. The plasma-adjusted concentration can be effective to inhibit α_{V}β₁ by at least about 80%. The plasma-adjusted concentration can be effective to inhibit α_{V}β₁ by at least about 90%. The recitation “percentage of each of α_{V}β₆ and/or α_{V}β₁ in the subject, each percentage independently selected” means, in the alternative, a single α_{V}β₆ inhibitor and corresponding percentage, a single α_{V}β₁ inhibitor and corresponding percentage, or a dual α_{V}β₆/α_{V}β₆ inhibitor and corresponding independently selected percentages.

Also provided herein are dosage forms configured for daily administration, comprising a pharmaceutically acceptable carrier or excipient; and a unit dose of an integrin modulating compound, such as compound 5, or a pharmaceutically acceptable salt thereof.

In various embodiments, a dose, e.g., a unit dose, such as a unit dose for daily administration, can include the compound in an amount of one of, or one of about: 1, 2.5, 5, 7.5, 10, 15, 20, 25, 30, 35, 40, 45, 50, 55, 60, 75, 80, 85, 90, 95, 100, 105, 110, 115, 120, 125, 130, 140, 150, 160, 170, 175, 180, 190, 200, 225, 240, 250, 275, 300, 320, 325, 350, 375, 400, 425, 450, 475, 480, 500, 525, 550, 560, 575, 600, 625, 640, 650, 675, 700, 720, 725, 750, 775, 800, 825, 850, 875, 880, 900, 925, 950, 960, 975, 1000, 1025, or 1040 milligrams. For example, a dose can include the compound in an amount of, or of about, 10 mg. A dose can include the compound in an amount of, or of about, 15 mg. A dose can include the compound in an amount of, or of about, 20 mg. A dose can include the compound in an amount of, or of about, 30 mg. A dose can include the compound in an amount of, or of about, 40 mg. A dose can include the compound in an amount of, or of about, 50 mg. A dose can include the compound in an amount of, or of about, 75 mg. A dose can include the compound in an amount of, or of about, 80 mg. A dose can include the compound in an amount of, or of about, 100 mg. A dose can include the compound in an amount of, or of about, 120 mg. A dose can include the compound in an amount of, or of about, 160 mg. A dose can include the compound in an amount of, or of about, 240 mg. A dose can include the compound in an amount of, or of about, 320 mg. A dose can include the compound in an amount of, or of about, 400 mg. A dose can include the compound in an amount of, or of about, 480 mg. A dose can include the compound in an amount of, or of about, 560 mg. A dose can include the compound in an amount of, or of about, 640 mg. A dose can include the compound in an amount of, or of about, 720 mg. A dose can include the compound in an amount of, or of about, 800 mg. A dose can include the compound in an amount of, or of about, 880 mg. A dose can include the compound in an amount of, or of about, 960 mg. A dose can include the compound in an amount of, or of about, 1040 mg.

In various embodiments, a dose, e.g., a unit dose, such as a unit dose for daily administration, can include the compound in an amount comprising an amount of the compound in mg of about one of about: 320, 400, 480, 560, 640, 720, 800, 880, 960, or 1040, or a range between any two of the preceding values.

In various embodiments, a dose, e.g., a unit dose, such as a unit dose for daily administration, can include the compound in an amount comprising an amount of the compound in mg of about one of about: 400, 480, 560, 640, 720, 800, 880, 960, or 1040.

In various embodiments, a dose, e.g., a unit dose, such as a unit dose for daily administration, can include the compound in an amount comprising an amount of the compound in mg of a range between about 320 and any one of about 400, 480, 560, 640, 720, 800, 880, 960, or 1040.

In various embodiments, a dose, e.g., a unit dose, such as a unit dose for daily administration, can include the compound in an amount comprising an amount of the compound in mg of about one of: 400, 480, 560, 640, 720, 800, 880, 960, or 1040, or a range between any two of the preceding values.

In some embodiments, the unit dose may include the compound in a percentage range about any of the individual values in milligrams recited in the preceding paragraph, for example, any percentage range independently selected from one of, or one of about: ±1%, ±2%, ±2.5%, ±5%, ±7.5%, ±10%, ±15%, ±20%, ±25%, ±30%, ±40%, or 50%. For example, the range may be, or be about, ±1%. The range may be, or be about, ±2%. The range may be, or be about, ±2.5%. The range may be, or be about, ±5%. The range may be, or be about, ±7.5%. The range may be, or be about, ±10%. The range may be, or be about, ±15%. The range may be, or be about, ±20%. The range may be, or be about, ±25%. The range may be, or be about, ±30%.

The range may be, or be about, ±40%. The range may be, or be about, ±50%.

Further, for example, the unit dose may include the compound in an amount of one of: 10 mg±1%; 10 mg 2%; 10 mg 2.5%; 10 mg 5%; 10 mg 7.5%; 10 mg 10%; 10 mg±15%; 10 mg±20%; 10 mg±25%; 10 mg±30%; 10 mg±40%; or 10 mg±50%. The unit dose may include the compound in an amount of one of: 15 mg±1%; 15 mg±2%; 15 mg±2.5%; 15 mg±5%; 15 mg±7.5%; 15 mg±10%; 15 mg±15%; 15 mg±20%; 15 mg±25%; 15 mg+30%; 15 mg±40%; or 15 mg±50%. The unit dose may include the compound in an amount of one of: 20 mg±1%; 20 mg±2%; 20 mg±2.5%; 20 mg±5%; 20 mg±7.5%; 20 mg±10%; 20 mg±15%; 20 mg±20%; 20 mg±25%; 20 mg±30%; 20 mg±40%; or 20 mg±50%. The unit dose may include the compound in an amount of one of: 30 mg±1%; 30 mg±2%; 30 mg±2.5%; 30 mg±5%; 30 mg±7.5%; 30 mg±10%; 30 mg±15%; 30 mg±20%; 30 mg±25%; 30 mg±30%; 30 mg±40%; or 30 mg±50%. The unit dose may include the compound in an amount of one of: 40 mg±1%; 40 mg±2%; 40 mg±2.5%; 40 mg±5%; 40 mg±7.5%; 40 mg 10%; 40 mg±15%; 40 mg±20%; 40 mg±25%; 40 mg±30%; 40 mg±40%; or 40 mg 50%. The unit dose may include the compound in an amount of one of: 50 mg±1%; 50 mg 2%; 50 mg 2.5%; 50 mg 5%; 50 mg 7.5%; 50 mg±10%; 50 mg 15%; 50 mg 20%; 50 mg±25%; 50 mg±30%; 50 mg±40%; or 50 mg±50%. The unit dose may include the compound in an amount of one of: 60 mg±1%; 60 mg±2%; 60 mg±2.5%; 60 mg±5%; 60 mg 7.5%; 60 mg±10%; 60 mg±15%; 60 mg 20%; 60 mg 25%; 60 mg 30%; 60 mg±40%; or 60 mg±50%. The unit dose may include the compound in an amount of one of: 75 mg±1%; 75 mg±2%; 75 mg±2.5%; 75 mg±5%; 75 mg±7.5%; 75 mg±10%; 75 mg±15%; 75 mg±20%; 75 mg±25%; 75 mg±30%; 75 mg±40%; or 75 mg±50%. The unit dose may include the compound in an amount of one of: 80 mg±1%; 80 mg±2%; 80 mg±2.5%; 80 mg±5%; 80 mg±7.5%; 80 mg±10%; 80 mg±15%; 80 mg±20%; 80 mg±25%; 80 mg±30%; 80 mg±40%; or 80 mg±50%. The unit dose may include the compound in an amount of one of: 100 mg 1%; 100 mg 2%; 100 mg 2.5%; 100 mg 5%; 100 mg 7.5%; 100 mg±10%; 100 mg±15%; 100 mg 20%; 100 mg 25%; 100 mg 30%; 100 mg 40%; or 100 mg±50%. The unit dose may include the compound in an amount of one of: 120 mg±1%; 120 mg 2%; 120 mg 2.5%; 120 mg 5%; 120 mg 7.5%; 120 mg 10%; 120 mg±15%; 120 mg 20%; 120 mg±25%; 120 mg 30%; 120 mg±40%; or 120 mg±50%. The unit dose may include the compound in an amount of one of: 160 mg±1%; 160 mg±2%; 160 mg±2.5%; 160 mg 5%; 160 mg 7.5%; 160 mg±10%; 160 mg 15%; 160 mg 20%; 160 mg 25%; 160 mg±30%; 160 mg±40%; or 160 mg±50%. The unit dose may include the compound in an amount of one of: 240 mg±1%; 240 mg±2%; 240 mg±2.5%; 240 mg 5%; 240 mg±7.5%; 240 mg±10%; 240 mg±15%; 240 mg±20%; 240 mg±25%; 240 mg 30%; 240 mg±40%; or 240 mg±50%. The unit dose may include the compound in an amount of one of: 320 mg 1%; 320 mg±2%; 320 mg±2.5%; 320 mg±5%; 320 mg±7.5%; 320 mg±10%; 320 mg±15%; 320 mg 20%; 320 mg 25%; 320 mg 30%; 320 mg 40%; or 320 mg 50%. The unit dose may include the compound in an amount of one of: 400 mg±1%; 400 mg±2%; 400 mg 2.5%; 400 mg 5%; 400 mg 7.5%; 400 mg±10%; 400 mg±15%; 400 mg±20%; 400 mg±25%; 400 mg 30%; 400 mg 40%; or 400 mg±50%. The unit dose may include the compound in an amount of one of: 480 mg±1%; 480 mg±2%; 480 mg±2.5%; 480 mg 5%; 480 mg±7.5%; 480 mg±10%; 480 mg±15%; 480 mg±20%; 480 mg±25%; 480 mg 30%; 480 mg±40%; or 480 mg 50%. The unit dose may include the compound in an amount of one of: 560 mg±1%; 560 mg 2%; 560 mg±2.5%; 560 mg±5%; 560 mg±7.5%; 560 mg±10%; 560 mg±15%; 560 mg±20%; 560 mg±25%; 560 mg±30%; 560 mg 40%; or 560 mg±50%. The unit dose may include the compound in an amount of one of: 640 mg±1%; 640 mg 2%; 640 mg±2.5%; 640 mg 5%; 640 mg±7.5%; 640 mg±10%; 640 mg±15%; 640 mg 20%; 640 mg±25%; 640 mg 30%; 640 mg±40%; or 640 mg±50%. The unit dose may include the compound in an amount of one of: 720 mg±1%; 720 mg±2%; 720 mg±2.5%; 720 mg±5%; 720 mg 7.5%; 720 mg±10%; 720 mg±15%; 720 mg 20%; 720 mg 25%; 720 mg±30%; 720 mg±40%; or 720 mg±50%. The unit dose may include the compound in an amount of one of: 800 mg±1%; 800 mg±2%; 800 mg±2.5%; 800 mg 5%; 800 mg±7.5%; 800 mg±10%; 800 mg±15%; 800 mg±20%; 800 mg±25%; 800 mg 30%; 800 mg±40%; or 800 mg±50%. The unit dose may include the compound in an amount of one of: 880 mg 1%; 880 mg±2%; 880 mg±2.5%; 880 mg±5%; 880 mg±7.5%; 880 mg±10%; 880 mg±15%; 880 mg 20%; 880 mg 25%; 880 mg 30%; 880 mg 40%; or 880 mg 50%. The unit dose may include the compound in an amount of one of: 960 mg±1%; 960 mg±2%; 960 mg±2.5%; 960 mg 5%; 960 mg 7.5%; 960 mg±10%; 960 mg±15%; 960 mg±20%; 960 mg±25%; 960 mg 30%; 960 mg 40%; or 960 mg±50%. The unit dose may include the compound in an amount of one of: 1040 mg±1%; 1040 mg±2%; 1040 mg±2.5%; 1040 mg 5%; 1040 mg 7.5%; 1040 mg±10%; 1040 mg 15%; 1040 mg 20%; 1040 mg 25%; 1040 mg 30%; 1040 mg 40%; or 1040 mg 50%.

A unit dose, such as a unit dose for daily administration, can comprise the compound in an amount effective on administration to an individual to produce a Cₘₐₓ in plasma of the individual in ng/mL of at least about, or greater than about, one of: 700, 750, 800, 850, 900, 950, 1000, 1050, 1100, 1150, 1200, 1250, 1300, 1350, 1400, 1450, 1500, 1600, 1700, 1800, 1900, 2000, 2100, 2200, 2300, 2400, or 2500; or a range between any two of the preceding concentrations, such as 700-1500, 700-900, 800-1300, 750-950, 800-1000, 850-950, 850-1050, 900-1400, 900-1300, 900-1200, 900-1100, 950-1050, 950-1400, 950-1150, 1000-1400, 1000-1300, 1000-1200, 700-2500, 1000-2500, 1500-2500, 1500-2000, 1500-2500, 2000-2500, and the like. For example, Cₘₐₓ can be, or be about, about 700 ng/mL or greater. Cₘₐₓ can be, or be about, about 750 ng/mL or greater. Cₘₐₓ can be, or be about, about 800 ng/mL or greater. Cₘₐₓ can be, or be about, 850 ng/mL or greater. Cₘₐₓ can be, or be about, 900 ng/mL or greater. Cₘₐₓ can be, or be about, 950 ng/mL or greater. Cₘₐₓ can be, or be about, 1000 ng/mL or greater. Cₘₐₓ can be, or be about, 1050 ng/mL or greater. Cₘₐₓ can be, or be about, 1100 ng/mL or greater. Cₘₐₓ can be, or be about, 1200 ng/mL or greater. Cₘₐₓ can be, or be about, 1300 ng/mL or greater. Cₘₐₓ can be, or be about, 1400 ng/mL or greater. Cₘₐₓ can be, or be about, 1500 ng/mL or greater. Cₘₐₓ can be, or be about, 1600 ng/mL or greater. Cₘₐₓ can be, or be about, 1700 ng/mL or greater. Cₘₐₓ can be, or be about, 1800 ng/mL or greater. Cₘₐₓ can be, or be about, 1900 ng/mL or greater. Cₘₐₓ can be, or be about, 2000 ng/mL or greater. Cₘₐₓ can be, or be about, 2100 ng/mL or greater. Cₘₐₓ can be, or be about, 2200 ng/mL or greater. Cₘₐₓ can be, or be about, 2300 ng/mL or greater. Cₘₐₓ can be, or be about, 2400 ng/mL or greater. Cₘₐₓ can be, or be about, 2500 ng/mL or greater.

A unit dose, such as a unit dose for daily administration, can comprise the compound in an amount effective on administration to an individual to produce a Cₘₐₓ in plasma of the individual in ng/mL of at least about one of 700, 750, 800, 850, 900, 950, 1000, 1050, 1100, 1150, 1200, 1250, 1300, 1350, 1400, 1450, or 1500, or a range between any two of the preceding concentrations

A unit dose, such as a unit dose for daily administration, can comprise the compound in an amount effective on administration to an individual to produce a Cₘₐₓ in plasma of the individual in ng/mL in a range between of at least about any one of 700, 750, 800, 850, 900, 950, 1000, 1050, 1100, 1150, 1200, 1250, 1300, 1350, 1400, or 1450 as a lower limit and 1500 as an upper limit.

A unit dose, such as a unit dose for daily administration, can comprise the compound in an amount effective on administration to an individual to produce a Cₘₐₓ in plasma of the individual in ng/mL of at least about one of: 1500, 1600, 1700, 1800, 1900, 2000, 2100, 2200, 2300, 2400, or 2500, or a range between any two of the preceding concentrations.

A unit dose, such as a unit dose for daily administration, can comprise the compound in an amount effective on administration to an individual to produce a Cₘₐₓ in plasma of the individual in ng/mL of at least about one of: 1600, 1700, 1800, 1900, 2000, 2100, 2200, 2300, 2400, or 2500, or a range between any two of the preceding concentrations.

A unit dose, such as a unit dose for daily administration, can comprise the compound in an amount effective on administration to an individual to produce a Cₘₐₓ in plasma of the individual in ng/mL in a range between at least 1500 and any one of 1600, 1700, 1800, 1900, 2000, 2100, 2200, 2300, 2400, or 2500.

A unit dose, such as a unit dose for daily administration, can comprise the compound in an amount effective on administration to an individual to produce a Cₘₐₓ in ng/mL in plasma of the individual, the Cₘₐₓ corresponding to a plasma-adjusted concentration effective to inhibit a percentage of α_{V}36 or α_{V}β₁ in the individual of at least one of, or at least about one of: 50, 55, 60, 65, 70, 75, 80, 85, 90, 95, 97, 98, 99, or 100, or a range between any two of the preceding percentages, for example, 50-100, 60-90, 70-90, 75-95, 90-95, 90-98, 90-99, and the like. In some embodiments, the compound may be a dual α_{V}β₆ and α_{V}β₁ inhibitor, and the Cₘₐₓ can correspond to a plasma-adjusted concentration effective to inhibit a percentage of each of α_{V}β₆ and α_{V}β₁ in the individual, each percentage independently selected from the preceding percentages, or a range between any two of the preceding percentages. For example, the plasma-adjusted concentration can be effective to inhibit α_{V}β₆ by at least about 50%. The plasma-adjusted concentration can be effective to inhibit α_{V}β₆ by at least about 60%. The plasma-adjusted concentration can be effective to inhibit α_{V}β₆ by at least about 70%. The plasma-adjusted concentration can be effective to inhibit α_{V}β₆ by at least about 80%. The plasma-adjusted concentration can be effective to inhibit α_{V}β₆ by at least about 90%. The plasma-adjusted concentration can be effective to inhibit α_{V}β₆ by at least about 95%. The plasma-adjusted concentration can be effective to inhibit α_{V}β₆ by at least about 97%. The plasma-adjusted concentration can be effective to inhibit α_{V}β₆ by at least about 98%. The plasma-adjusted concentration can be effective to inhibit α_{V}β₆ by at least about 99%. The plasma-adjusted concentration can be effective to inhibit α_{V}β₆ by about 100%. Further, for example, the plasma-adjusted concentration can be effective to inhibit α_{V}β₁ by at least about 50%. The plasma-adjusted concentration can be effective to inhibit α_{V}β₁ by at least about 60%. The plasma-adjusted concentration can be effective to inhibit α_{V}β₁ by at least about 70%. The plasma-adjusted concentration can be effective to inhibit α_{V}β₁ by at least about 80%. The plasma-adjusted concentration can be effective to inhibit α_{V}β₁ by at least about 90%. The plasma-adjusted concentration can be effective to inhibit α_{V}β₁ by at least about 95%. The plasma-adjusted concentration can be effective to inhibit α_{V}β₁ by at least about 97%. The plasma-adjusted concentration can be effective to inhibit α_{V}β₁ by at least about 98%. The plasma-adjusted concentration can be effective to inhibit α_{V}β₁ by at least about 99%. The plasma-adjusted concentration can be effective to inhibit α_{V}β₁ by about 100%. The recitation “percentage of each of α_{V}β₆ and/or α_{V}β₁ in the subject, each percentage independently selected” means, in the alternative, a single α_{V}β₆ inhibitor and corresponding percentage, a single α_{V}β₁ inhibitor and corresponding percentage, or a dual α_{V}β₆/α_{V}β₆ inhibitor and corresponding independently selected percentages.

The dosage form for daily administration can be administered to an individual in need thereof once daily. That is, the total amount of an integrin modulating compound, such as compound 5, or a pharmaceutically acceptable salt thereof, which is to be administered each day, can be administered all together at one time daily. Alternatively, if it is desirable that the total amount of an integrin modulating compound, such as compound 5, or a pharmaceutically acceptable salt thereof, is to be administered in two or more portions daily, the dosage form containing the appropriate amount of compound can be administered two times or more daily, such as twice a day, three times a day, or four times a day.

### Kits

The invention further provides kits for carrying out the methods of the invention, which comprises one or more compounds described herein, or a salt thereof, or a pharmacological composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for use in the treatment of a fibrotic disease.

Kits generally comprise suitable packaging. The kits may comprise one or more containers comprising any compound described herein. Each component (if there is more than one component) can be packaged in separate containers or some components can be combined in one container where cross-reactivity and shelf life permit. One or more components of a kit may be sterile and/or may be contained within sterile packaging.

The kits may be in unit dosage forms, bulk packages (e.g., multi-dose packages) or sub-unit doses. For example, kits may be provided that contain sufficient dosages of a compound as disclosed herein (e.g., a therapeutically effective amount) and/or a second pharmaceutically active compound useful for a disease detailed herein (e.g., fibrosis) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The kits may optionally further comprise instructions for daily administration of the dosage form to an individual in need thereof, such as instructions for administration of the dosage form to an individual in need thereof one, two, three, or four times daily, for example, instructions for administration of the dosage form to an individual in need thereof once daily.

### ENUMERATED EMBODIMENTS

The following enumerated embodiments are representative of some aspects of the invention.

Embodiment A-1. A method of therapy for a condition mediated by at least one integrin, comprising:

providing a subject in need of therapy, the subject comprising: the condition mediated by the at least one integrin, and a tissue involved in the condition that expresses the at least one integrin;

modulating the at least one integrin in the tissue in the subject effective to treat the condition, comprising administering to the subject at least one compound that binds to a receptor of the at least one integrin, the at least one compound being administered to the subject in an amount effective to achieve a percent occupancy of the receptor of the at least one integrin in the tissue of the subject of at least about 50%.

Embodiment A-2. The method of Embodiment A-1, the at least one compound being administered to the subject in an amount effective to achieve a percent occupancy of the receptor in the tissue of the subject of about one of, or a range between about any two of: 50%, 55%, 60%, 65%, 70%, 75%, 80%, 85%, 90%, 95%, 96%, 97%, 98%, 99%, or 100%.
Embodiment A-3. The method of Embodiment A-1, wherein the compound is orally administered to the subject.
Embodiment A-4. The method of Embodiment A-1, wherein the percent occupancy of the receptor in the tissue of the subject is achieved using a single dose of the compound.
Embodiment A-5. The method of Embodiment A-1, wherein the compound inhibits the at least one integrin in the subject effective to treat the condition.
Embodiment A-6. The method of Embodiment A-1, wherein the tissue has at least one elevated level selected from the group consisting of:
activity and/or expression of the at least one integrin;
a pSMAD/SMAD value;
new collagen formation or accumulation;
total collagen; and
Type I Collagen gene Col1a1 expression;
   wherein the level is elevated compared to a healthy state of the tissue.
   Embodiment A-7. The method of Embodiment A-6, wherein the elevated pSMAD/SMAD value is at least one of an elevated pSMAD2/SMAD2 value or an elevated pSMAD3/SMAD3 value.
   Embodiment A-8. The method of Embodiment A-1, wherein the tissue is selected from at least one member of the group consisting of: lung tissue, liver tissue, skin tissue, cardiac tissue, kidney tissue, gastrointestinal tissue, gall bladder tissue, and bile duct tissue.
   Embodiment A-9. The method of Embodiment A-1, wherein the at least one integrin comprises an α_{V} subunit.
   Embodiment A-10. The method of Embodiment A-1, wherein the at least one integrin comprises a β₁ or a β₆ subunit.
   Embodiment A-11. The method of Embodiment A-1, wherein the at least one integrin comprises at least one of: α_{V}β₁ integrin and α_{V}β₆ integrin.
   Embodiment A-12. The method of Embodiment A-1, wherein the at least one integrin comprises α_{V}β₁ integrin and α_{V}β₆ integrin.
   Embodiment A-13. The method of Embodiment A-1, wherein the condition mediated by the at least one integrin is selected from the group consisting of: a fibrotic disease and psoriasis.
   Embodiment A-14. The method of Embodiment A-13, wherein the condition mediated by the at least one integrin is a fibrotic disease selected from the group consisting of: idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, scleroderma, diabetic nephropathy, diabetic kidney disease, focal segmental glomerulosclerosis, chronic kidney disease, and Crohn's Disease.
   Embodiment A-15. The method of Embodiment A-14, wherein the compound inhibits the activity of one or both of α_{V}β₁ integrin and α_{V}β₆ integrin in the subject, thereby treating the fibrotic disease in the subject.
   Embodiment A-16. The method of Embodiment A-14, wherein the condition mediated by the at least one integrin is NASH, and the compound inhibits the activity of at least α_{V}β₁ integrin in the subject effective to treat the subject for NASH.
   Embodiment A-17. The method of Embodiment A-14, wherein the condition mediated by the at least one integrin is IPF, and the compound inhibits the activity of at least α_{V}β₆ integrin in the subject effective to treat the subject for IPF.
   Embodiment A-18. The method of Embodiment A-17, wherein the compound inhibits the activity of α_{V}β₁ integrin and α_{V}β₆ integrin in the subject effective to treat the subject for IPF.
   Embodiment A-19. The method of Embodiment A-14, wherein the condition mediated by the at least one integrin is PSC, and the compound inhibits the activity of at least one of α_{V}β₆ integrin and α_{V}β₁ integrin in in the subject effective to treat the subject for PSC.
   Embodiment A-20. The method of Embodiment A-19, wherein the compound inhibits the activity of α_{V}β₁ integrin and α_{V}β₆ integrin in the subject effective to treat the subject for PSC.
   Embodiment A-21. The method of Embodiment A-14, wherein the condition mediated by the at least one integrin is psoriasis.
   Embodiment A-22. The method of Embodiment A-21, wherein the condition mediated by the at least one integrin is psoriasis, and the compound inhibits the activity of one or both of α_{V}β₁ integrin and α_{V}β₆ integrin in the subject effective to treat the subject for psoriasis.
   Embodiment A-23. The method of Embodiment A-1, wherein the method selectively reduces α_{V}β₁ integrin activity and/or expression in the tissue compared to at least one other α_{V}-containing integrin in the subject.
   Embodiment A-24. The method of Embodiment A-23, wherein the method selectively reduces α_{V}β₁ integrin activity and/or expression in the tissue compared to α_{V}β₆ integrin in the subject.
   Embodiment A-25. The method of Embodiment A-1, wherein the method selectively reduces α_{V}β₆ integrin activity and/or expression in the tissue compared to at least one other α_{V}-containing integrin in the subject.
   Embodiment A-26. The method of Embodiment A-25, wherein the method selectively reduces α_{V}β₆ integrin activity and/or expression in the tissue compared to α_{V}β₁ integrin in the subject.
   Embodiment A-27. The method of Embodiment A-1, wherein the method selectively reduces α_{V}β₆ integrin or α_{V}β₁ integrin activity and/or expression in the tissue compared to at least one other α_{V}-containing integrin in the subject.
   Embodiment A-28. The method of Embodiment A-1, wherein the method selectively reduces α_{V}β₆ integrin and α_{V}β₁ integrin activity and/or expression in the tissue compared to at least one other α_{V}-containing integrin in the subject.
   Embodiment A-29. The method of Embodiment A-1, wherein the tissue comprises one or more fibroblasts, and the method inhibits α_{V}β₁ integrin in the one or more fibroblasts.
   Embodiment A-30. The method of Embodiment A-1, wherein the tissue comprises one or more epithelial cells, and the method inhibits α_{V}β₆ integrin in the one or more epithelial cells.
   Embodiment A-31. The method of Embodiment A-1, wherein a dose of the at least one compound administered to the subject is an amount in milligrams selected from about one of, or at least about one of: 10, 15, 20, 30, 40, 50, 60, 75, 80, 100, 120, 160, 240, 320, 400, 480, 560, 640, 720, 800, 880, 960, or 1040, or a range between any two of the preceding values.
   Embodiment A-32. The method of Embodiment A-1, wherein a dose of the at least one compound administered to the subject is an amount in milligrams selected from about one of, or at least about one of: 60,120,240, and 320, or a range between any two of the preceding values.
   Embodiment A-33. The method of Embodiment A-1, comprising administering the compound to the subject effective to produce an unbound plasma concentration in nM of the compound in the subject selected from about one of, or at least about one of: 1, 10, 15, 20, 25, 30, 40, 50, 60, 75, 80, 90, 100, 125, 150, 200, 250, 500, 750, 1000, or 1250, or a range between any two of the preceding values.
   Embodiment A-34. The method of Embodiment A-1, comprising administering the compound to the subject effective to produce an unbound plasma concentration in nM of the compound in the subject selected from about one of, or at least about one of: 10, 15, 20, 25, 30, 40, 50, 60, 75, 80, 90, 100, or 125, or a range between any two of the preceding values.
   Embodiment A-35. The method of any one of Embodiments A-1 to A-34, wherein the compound is (S)-4-((2-methoxyethyl)(4-(5,6,7,8-tetrahydro-1,8-naphthyridin-2-yl)butyl)amino)-2-(quinazolin-4-ylamino)butanoic acid:

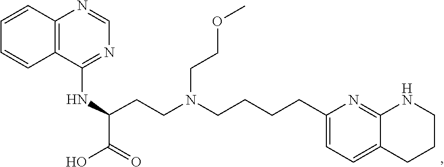 [SMILES: COCCN(CCCCc1ccc2c(n1)NCCC2)CC[C@H](Nc1ncnc2ccccc12)C(=O)O]

or a salt thereof.
Embodiment A-36. The method of any one of Embodiments A-1 to A-35, wherein the compound is administered to the subject in a dosage form configured for daily administration, the dosage form comprising a pharmaceutically acceptable carrier or excipient and a unit dose of the compound, or a salt thereof.
Embodiment A-37. The method of Embodiment A-36, the dosage form comprising about 1, 2.5, 5, 7.5, 10, 15, 20, 25, 30, 35, 40, 50, 75, 80, 85, 90, 95, 100, 105, 110, 115, 120, or 125 mg of the compound, or a range between any two of the preceding values.
Embodiment A-38. The method of Embodiment A-36, the dosage form comprising the compound in mg of about one of: 1, 2.5, 5, 7.5, 10, 15, 20, 25, 30, 35, 40, 50, 60, 75, 80, 85, 90, 95, 100, 105, 110, 115, 120, 125, 150, 175, 200, 225, or 250, or a range between any two of the preceding amounts.
Embodiment A-39. The method of Embodiment A-36, the dosage form comprising the compound in mg of about one of: 10, 15, 20, 30, 40, 50, 75, 80, 100, 120, 160, 240, or 320, or a range between any two of the preceding values.
Embodiment A-40. The method of Embodiment A-36, the dosage form comprising the compound in mg of about one of: 320, 400, 480, 560, 640, 720, 800, 880, 960, or 1040, or a range between any two of the preceding values.
Embodiment A-41. The method of Embodiment A-36, the dosage form comprising the compound in in an amount effective on administration to an individual to produce a Cₘₐₓ in plasma of the individual in ng/mL of at least about one of 700, 750, 800, 850, 900, 950, 1000, 1050, 1100, 1150, 1200, 1250, 1300, 1350, 1400, 1450, or 1500, or a range between any two of the preceding concentrations.
Embodiment A-42. The method of Embodiment A-36, the dosage form comprising the compound in an amount effective on administration to an individual to produce a Cₘₐₓ in plasma of the individual in ng/mL of at least about one of: 1500, 1600, 1700, 1800, 1900, 2000, 2100, 2200, 2300, 2400, or 2500, or a range between any two of the preceding concentrations.
Embodiment A-43. The method of Embodiment A-36, the dosage form comprising the compound in an amount effective on administration to an individual to produce a Cₘₐₓ in ng/mL in plasma of the individual, the Cₘₐₓ corresponding to a plasma-adjusted concentration effective to inhibit a percentage of α_{V}β₆ or α_{V}β₁ in the individual of at least about one of 50, 55, 60, 65, 70, 75, 80, 85, 90, 95, or 100, or a range between any two of the preceding percentages.
Embodiment A-44. The method of Embodiment A-36, the dosage form configured for daily administration, the method comprising daily administration of the dosage form to the subject.
Embodiment A-45. The method of Embodiment A-36, the dosage form configured for daily administration, the method comprising administration of the dosage form to the subject one, two, three, or four times daily.

### EXAMPLES

### Example B1—α_{V}β₆ and α_{V}β₁ Inhibition Activity Shown in Normal Human Bronchial Epithelial Cells and IPF-Derived Human Lung Fibroblasts

Two latency associated peptide (LAP) adhesion binding assays were devised using primary human lung cells, including normal (healthy) human bronchial epithelial cells and human lung fibroblasts (healthy and IPF).

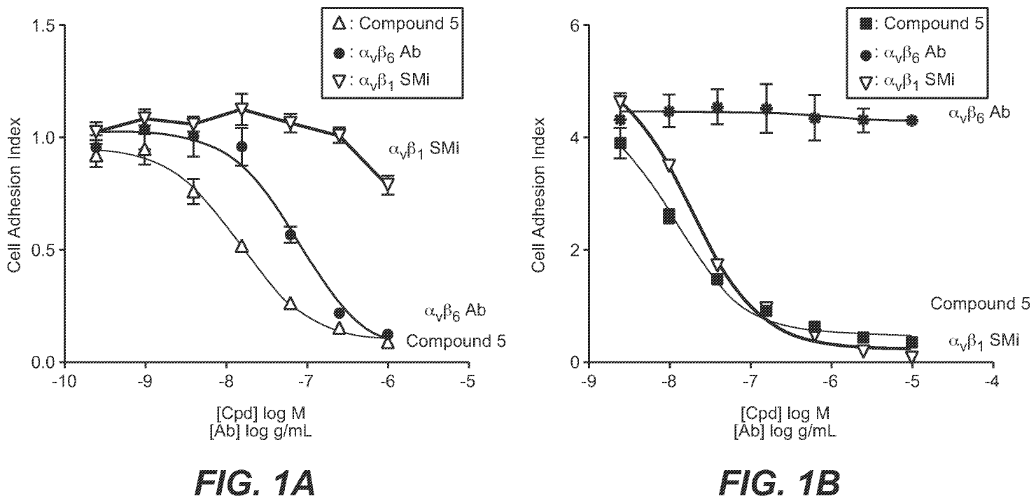

Human bronchial epithelial cells are known to express α_{V}β₆ integrin in culture. Human bronchial epithelial cells were prepared for the assay by dissociation with trypsin/EDTA and were then seeded at 20,000 cells per well on 96 well plates (ACEA Bioscience E-plate View, Acea Biosciences; San Diego, CA) previously coated with 5 μg/ml of recombinant human LAP (R&D Systems; Minneapolis, MN) and blocked with 4% bovine serum albumin. Cell index (electrical impedance) was measured to assess cell attachment/spreading every 3 minutes for 24 hours at 37° C./5% CO₂ using the xCELLigence RTCA MP Instrument (Acea Biosciences; San Diego, CA). EC₉₀ (time point at 90% of the peak cell index) was determined for vehicle-treated cells and IC₅₀ curves for test article-treated cells were generated at that time point. In the assay, the IPF-derived human bronchial epithelial cells were separately incubated with: a α_{V}β₁-selective small molecule inhibitor (characterized by sub-50 nM IC₅₀ for α_{V}β₁, and selective for α_{V}β₁ over α_{V}06 by a factor of about 25); a selective antibody α_{V}β₆ inhibitor, 3G9 (ITGB1BP2 Monoclonal Antibody (3G9), ThermoFisher Scientific, Santa Clara, CA); and compound 5, (S)-4-((2-methoxyethyl)(4-(5,6,7,8-tetrahydro-1,8-naphthyridin-2-yl)butyl) amino)-2-(quinazolin-4-ylamino)butanoic acid. FIG. 1A shows that compound 5 and the selective antibody α_{V}β₆ inhibitor 3G9 both substantially inhibited normal bronchial epithelial cell adhesion to LAP, in contrast with the α_{V}β₁-selective small molecule inhibitor.

Human lung fibroblasts derived from normal and IPF lung tissue are known to express α_{V}β₁ integrin. The IPF-derived human lung fibroblasts were prepared for the assay by dissociation with trypsin/EDTA, and were seeded at 20,000 cells per well on 96 well plates (ACEA Bioscience E-plate View, Acea Biosciences; San Diego, CA) previously coated with 5 g/ml of recombinant human LAP (R&D Systems; Minneapolis, MN) and blocked with 4% bovine serum albumin. Cell index (electrical impedance) was measured to assess cell attachment/spreading every 3 minutes for 24 hours at 37° C./5% CO₂ using the xCELLigence RTCA MP Instrument (Acea Biosciences; San Diego, CA). EC₉₀ (time point at 90% of the peak cell index) was determined for vehicle-treated cells and IC₅₀ curves for test article-treated cells were generated at that time point. In the assay, the IPF-derived human lung fibroblasts were separately incubated with: the α_{V}β₁-selective small molecule inhibitor; the selective antibody α_{V}β₆ inhibitor, 3G9; and compound 5. FIG. 1B shows that compound 5 and the α_{V}β₁-selective small molecule inhibitor both substantially inhibited cell adhesion in the IPF-derived lung fibroblasts, in contrast to the selective antibody α_{V}β₆ inhibitor, 3G9.

### Example B2—Dual α_{V}β₆/α_{V}β₁ Inhibition Reduces Collagen Deposition in the Murine Bleomycin Model

It has been previously shown that inhibition of α_{V}β₆ in the lung can be detected though measurement of phospho-SMAD (pSMAD) in alveolar macrophages. Alveolar macrophages are known to operate in a unique niche in the lung, distinct from interstitial macrophages. SMAD3 is a downstream target of the active TGF-β cytokine binding its receptor and in alveolar macrophages it is phosphorylated by homoeostatic levels of TGF-β. Accordingly, it was desirable to know whether inhibition of TGF-β activation using the disclosed compounds would result in reduced SMAD2 and SMAD3 phosphorylation.

Mice (C57BL/6) were divided into healthy (n=15), vehicle-treated (n=15), and test article-treated (n=15 per dose) groups. Mice in the vehicle and test article-treated groups were administered 3 U/kg of bleomycin (Teva Pharmaceuticals; North Wales, PA) via oropharyngeal aspiration while under anesthesia on day 0. Healthy animals were administered water in a similar fashion. Starting on day 7, mice in the control group were administered PBS vehicle, 130 μL, by oral gavage, BID for 14 days. Also starting on day 7, mice in the test group were administered compound 5 in PBS by oral gavage, BID for 14 days, at relative dosages of 1×, 2.5× and 5×. The absolute amount of the 1× dosage was selected at a value in mg/kg that showed significant efficacy. From day 14 through day 21, 9 of the 15 mice were administered ²H₂O for labeling. All mice were sacrificed on day 21 and tissues were collected. Samples were prepared for analysis either directly from lung tissue, or by bronchoalveolar lavage, which washes out the bronchiolar and alveolar space with saline to produce a bronchoalveolar lavage fluid (BALF) in which 80-90% of cells are alveolar macrophages.

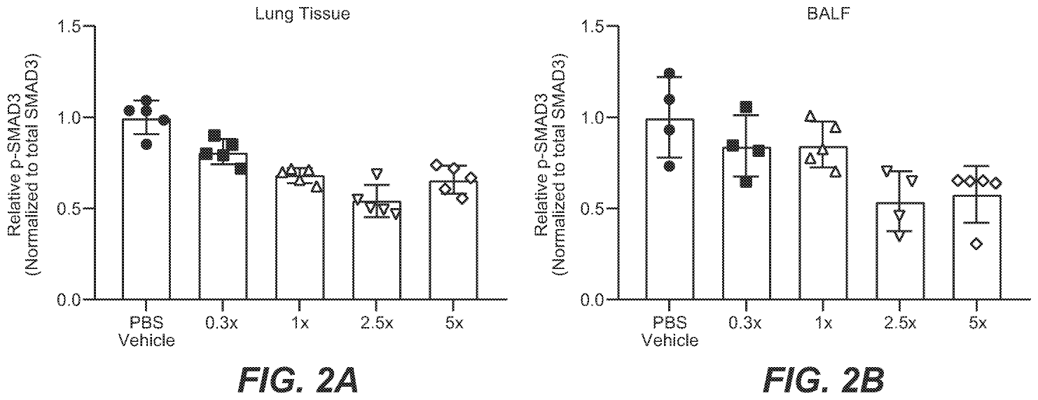

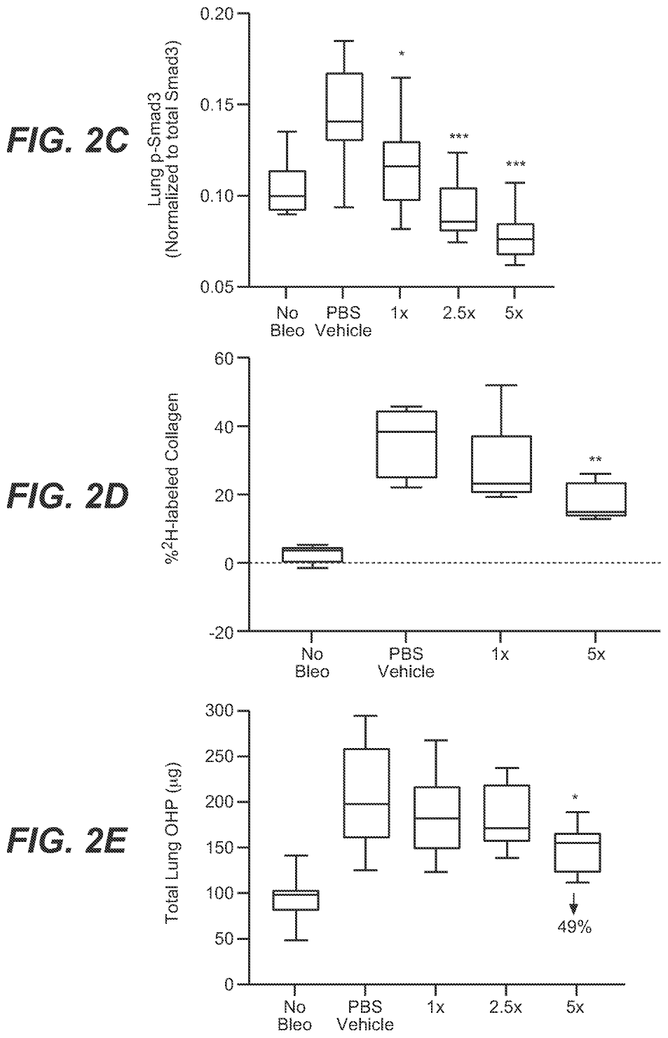

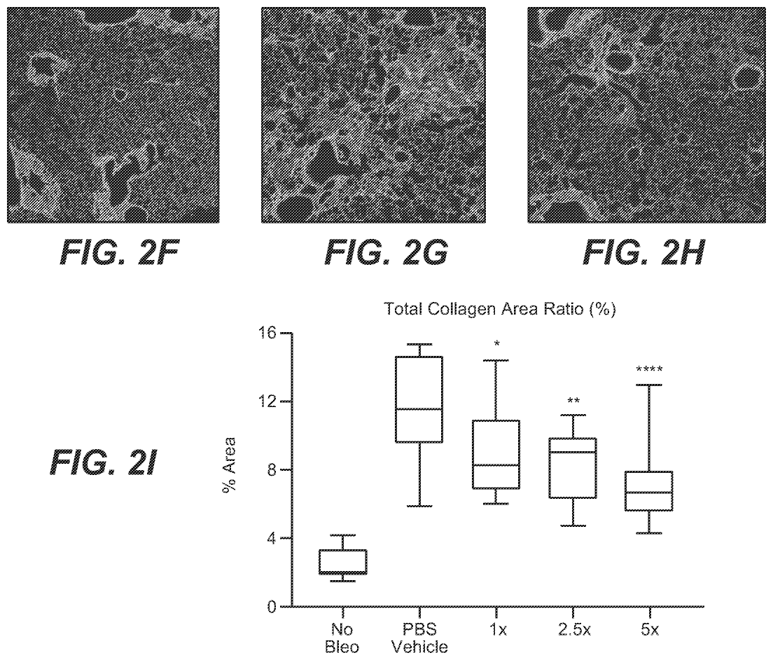

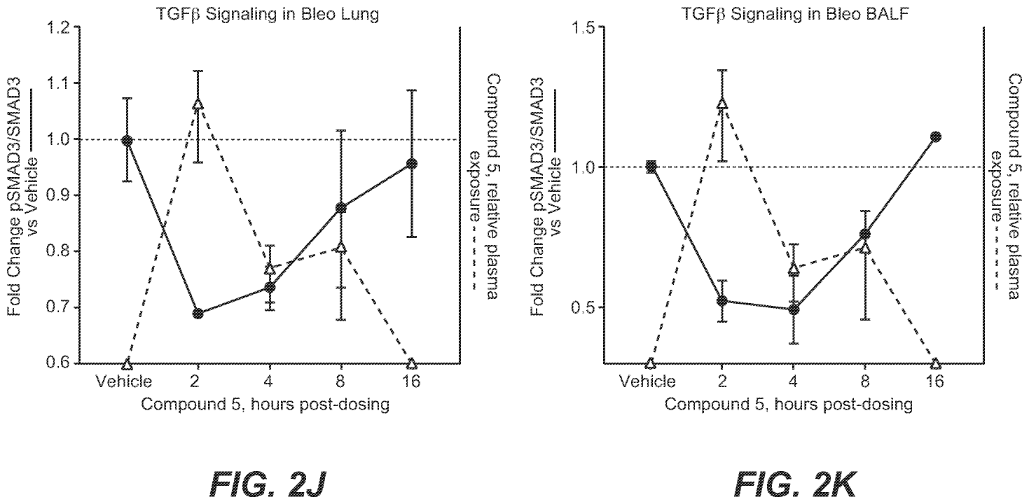

FIG. 2A is a graph of PSMAD3/SMAD3 in lung tissue from healthy mice administered PBS vehicle and varying levels of compound 5 for 4 days. FIG. 2B is a graph of PSMAD3/SMAD3 in BALF drawn from the same healthy mice administered PBS vehicle and varying levels of compound 5 for 4 days. FIGS. 2A and 2B show that 4 days of compound 5 treatment significantly reduced SMAD3 phosphorylation in both lung tissue and cells isolated from BALF in a dose dependent manner to approximately 50% of the untreated levels

FIG. 2C is a graph showing that compared to the healthy mice, lung tissue in the vehicle-treated mice experienced a substantial increase in SMAD3 phosphorylation, which is a measure of TGF-β signaling-related kinase activity. FIG. 2C also shows, compared to the vehicle-treated mice, substantial, statistically significant dose-dependent reductions in SMAD3 phosphorylation in the test article-treated mice according to the dosage of compound 5, including at 1× (p<0.05 vs vehicle), 2.5× (p<0.01 vs vehicle), and 5× mg/kg (p<0.001 vs vehicle). This time- and dose-dependent inhibition of pSMAD3 levels in the lung to approximately 50% of the untreated levels was associated with inhibition of fibrosis according to the following results. FIG. 2D is a graph showing that compared to the healthy mice, lung tissue in the vehicle-treated mice experienced a substantial accumulation of new collagen as evidenced by the percentage of lung collagen containing ²H-labeled hydroxyproline. FIG. 2D also shows, compared to the control mice, a dose-dependent reduction in accumulated new collagen as evidenced by the percentage of lung collagen containing ²H-labeled hydroxyproline in the test mice, including at 1×, and at 5× (p<0.01 vs vehicle). FIG. 2E shows that compared to the healthy mice, the vehicle-treated mice experienced a significant increase in total pulmonary collagen, as measured by μg of hydroxyproline. FIG. 2E also shows, compared to the control mice, a reduction in total pulmonary collagen in the test mice according to the dosage of compound 5, including at 1×, 2.5×, and 5× (p<0.05 vs vehicle). As shown among FIGS. 2C, 2D, and 2E, in fibrotic bleomycin-treated mice, compound 5 abrogated the increase in pSMAD3 due to bleomycin challenge, a reduction that was associated with inhibition of fibrosis.

FIGS. 2F, 2G, and 2H show high resolution second harmonic generation images of fibrillar collagen (collagen type I and III) taken from formalin-fixed paraffin embedded lung tissue sections from a healthy mouse lung (2F), a vehicle-treated mouse lung (2G) and a test-article treated mouse lung (2H; 500 mg/kg BID). Color scale is indicative of collagen fiber density (red=most dense; blue=least dense).

FIG. 2I is a graph showing the percent total collagen area in the second harmonic generation mouse lung images. Large structural areas of collagen found similarly in healthy and fibrotic tissues (dense collagen fibers surrounding airways were excluded from this analysis to focus on interstitial fibrotic collagen). FIG. 2I shows that compared to the healthy mice, lung tissue in the vehicle-treated mice experienced a substantial increase in total collagen area in the second harmonic generation images. FIG. 2I also shows that compared to the control mice, lung tissue in the test article-treated mice experienced a substantial, statistically significant dose-dependent reduction in total collagen area in the second harmonic generation images according to the administration of compound 5, including at 1× (p<0.05 vs vehicle), 2.5× (p<0.01 vs vehicle), and 5× (p<0.0001 vs vehicle). The 1×, 2.5×, and 5× dosages were at the same absolute values in mg/kg as in Example B2.

FIGS. 2J and 2K are graphs of sequential measurements in bleomycin-treated mice, which demonstrated a close inverse relationship between pSMAD3 levels in lung (2J) and BALF cells (2K) vs. plasma drug exposure. The data for FIGS. 2J and 2K, was obtained 14 days after bleomycin challenge in mice were treated with compound 5 at 2.5× dose (PO, BID for 1.5 days).

### Example B3—Dual α_{V}β₁/α_{V}β₆ Inhibition Outperforms Single Integrin Inhibition in Precision Cut Lung Slice Assays of Mice Under Acute Bleomycin Exposure

Mice (C57BL/6) were administered 3 U/kg of bleomycin (Teva Pharmaceuticals; North Wales, PA) on day 0 via oropharyngeal aspiration while under anesthesia. On day 14, precision cut lung slices were obtained. Following euthanization, 2% low gelling temp agarose was injected into the mouse lungs via the trachea. Lungs were excised and the inferior lobe separated by dissection. The lobes were then subjected to precision slicing to obtain samples for culture using a microtome (Compresstome VF-300-OZ, Precisionary; Greenville, NC). Individual slices were distributed in a multiwell culture plate and cultured for 3 days under control (DMSO) and test compound conditions. The viability of the slices over the course of culturing was confirmed by WST-1 assay of mitochondrial activity.

During the culture period, slices in the control group were treated with DMSO and slices in the test group were treated with a DMSO solution of one of: a selective antibody α_{V}β₆ inhibitor, 3G9; the α_{V}β₁-selective small molecule inhibitor; compound 5; a first pan-α_{V} small molecule inhibitor ((3S)-3-[3-(3,5-dimethyl-1H-pyrazol-1-yl)phenyl]-4-{(3S)-3-[2-(5,6,7,8-tetrahydro-1,8-naphthyridin-2-yl)ethyl]-1-pyrrolidinyl}butanoic acid, PROBECHEM®, St. Petersburg, FL); a second pan-α_{V} small molecule inhibitor ((3S)—N-[3-hydroxy-5-[(1,4,5,6-tetrahydro-5-hydroxy-2-pyrimidinyl)amino]benzoyl]glycyl-3-[3-bromo-5-(1,1-dimethylethyl)phenyl]-β-alanine, Cayman Chemical, Ann Arbor, MI); and a small molecule ALK5 (TGF-β type I receptor) inhibitor (4-[2-Fluoro-5-[3-(6-methyl-2-pyridinyl)-1H-pyrazol-4-yl]phenyl]-1H-pyrazole-1-ethanol, Bio-Techne Corporation, Minneapolis, MN). Single and dual integrin inhibitors were analyzed at their respective IC₅₀ concentrations for inhibition of TGF-beta activation (compound 5 run at IC₅₀ for α_{V}β₆). The pan α_{V} integrin inhibitors and small molecule ALK5 inhibitor were analyzed at concentrations 10× above their respective reported IC₅₀ values.

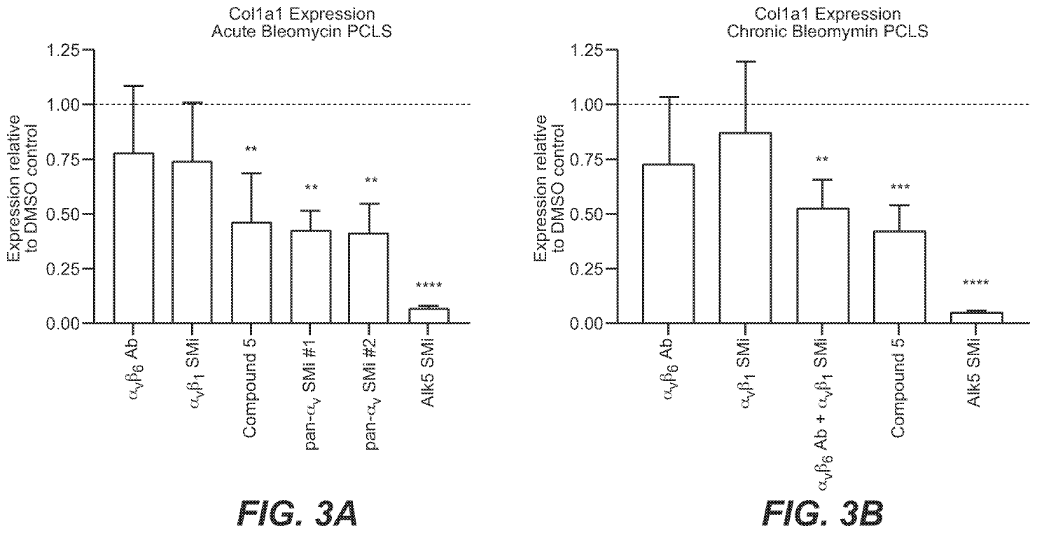

FIG. 3A is a bar graph, normalized to control slices treated with DMSO, showing that all test treatments reduced Type I Collagen gene Col1a1 expression, although selective antibody α_{V}β₆ inhibitor 3G9 and the α_{V}β₁-selective small molecule inhibitor were not statistically significant. Compound 5, as a dual α_{V}β₁/α_{V}β₆ inhibitor, decreased Type I Collagen gene Col1a1 expression substantially (p<0.01 vs vehicle) compared to the DMSO control, the selective antibody α_{V}β₆ inhibitor, 3G9; and the α_{V}β₁-selective small molecule inhibitor. Compound 5 decreased Type I Collagen gene Col1a1 expression comparably to the first and second pan-α_{V} small molecule inhibitors (each p<0.01 compared to the DMSO control). The small molecule ALK5 inhibitor, used as a positive control representative of total TGF-beta signaling inhibition, provided the greatest decrease in Type I Collagen gene Col1a1 expression (p<0.0001 compared to the DMSO control).

### Example B4-Dual α_{V}β₁/α_{V}β₆ Inhibition Outperforms Single Integrin Inhibition in Precision Cut Lung Slice Assays of Mice Under Chronic Bleomycin Exposure

Mice (C57BL/6) were administered 3 U/kg of bleomycin (Teva Pharmaceuticals; North Wales, PA) on Day 0 and 1 U/kg of bleomycin on days 14, 28, 42 and 56 via oropharyngeal aspiration while under anesthesia. At day 70, 14 days after the final bleomycin insult, precision cut lung slices were obtained. Following euthanization, 2% low gelling temp agarose was injected into the mouse lungs via the trachea. Lungs were excised and the inferior lobe separated by dissection. The lobes were then subjected to precision slicing to obtain samples for culture using a microtome (Compresstome VF-300-OZ, Precisionary; Greenville, NC). Individual slices were distributed in a multiwell culture plate and cultured for 7 days under control (DMSO) and test compound conditions. The viability of the slices over the course of culturing was confirmed by WST-1 assay of mitochondrial activity.

During the culture period, slices in the control group were treated with DMSO and slices in the test group were treated with a DMSO solution of one of: the selective antibody α_{V}β₆ inhibitor 3G9; the α_{V}β₁-selective small molecule inhibitor; a combination of the selective antibody α_{V}β₆ inhibitor 3G9 and the α_{V}β₁-selective small molecule inhibitor; compound 5; and the small molecule ALK5 inhibitor. The selective α_{V}β₁ and α_{V}β₆ integrin inhibitors were analyzed at ≥their respective IC₉₀ concentrations for inhibition of TGF-beta activation. Compound 5 was run at approximate IC₅₀ for inhibition of TGF-beta activation by α_{V}β₆. The small molecule ALK5 inhibitor was analyzed at 10× its reported IC₅₀ value.

FIG. 3B is a bar graph, normalized to control slices treated with DMSO, showing that all test treatments reduced lung Col1a1 expression. Compound 5, as a dual α_{V}β₁/α_{V}β₆ inhibitor, decreased lung Col1a1 expression substantially (p<0.01 vs vehicle) compared to the DMSO control, the selective antibody α_{V}β₆ inhibitor, 3G9; and the α_{V}β₁-selective small molecule inhibitor. Compound 5 also decreased lung Col1a1 expression to a greater extent than combined administration (p<0.001 vs vehicle) of the selective antibody α_{V}β₆ inhibitor 3G9 and the α_{V}β₁-selective small molecule α_{V}β₁ inhibitor. The small molecule ALK5 inhibitor, used as a positive control representative of total TGF-beta signaling inhibition, provided the greatest decrease in Type I Collagen gene Col1a1 expression (p<0.0001 compared to the DMSO control).

### Example B5-Dual α_{V}β₁/α_{V}β₆ Inhibition More Potently Blocks Collagen Gene Expression in the Murine Bleomycin Model than Pirfenidone and Nintedanib

Mice (C57BL/6) were administered 3 U/kg of bleomycin (Teva Pharmaceuticals; North Wales, PA) on Day 0 and 1 U/kg of bleomycin on days 14, 28, 42 and 56 via oropharyngeal aspiration while under anesthesia. At day 70, 14 days following the final bleomycin insult, precision cut lung slices were obtained. Following euthanization, 2% low gelling temp agarose was injected into the mouse lungs via the trachea. Lungs were excised and the inferior lobe separated by dissection. The lobes were then subjected to precision slicing to obtain samples for culture using a microtome (Compresstome VF-300-OZ, Precisionary; Greenville, NC). Individual slices were distributed in a multiwell culture plate and cultured for 7 days under control (DMSO) and test compound conditions. The viability of the slices over the course of culturing was confirmed by WST-1 assay of mitochondrial activity.

During the culture period, slices in the control group were treated with DMSO and slices in the test group were treated with a DMSO solution of one of: compound 5; nintedanib; pirfenidone; a combination of nintedanib and compound 5; a combination of pirfenidone and compound 5; or the small molecule ALK5 inhibitor. Compound 5 was administered to mice effective to equal or exceed its respective IC₅₀ values at α_{V}β₆ and α_{V}β₁. The small molecule ALK5 inhibitor was analyzed at 10× its reported IC₅₀ value. Nintendanib and pirfenidone were analyzed at concentrations 10× their reported therapeutic concentrations.

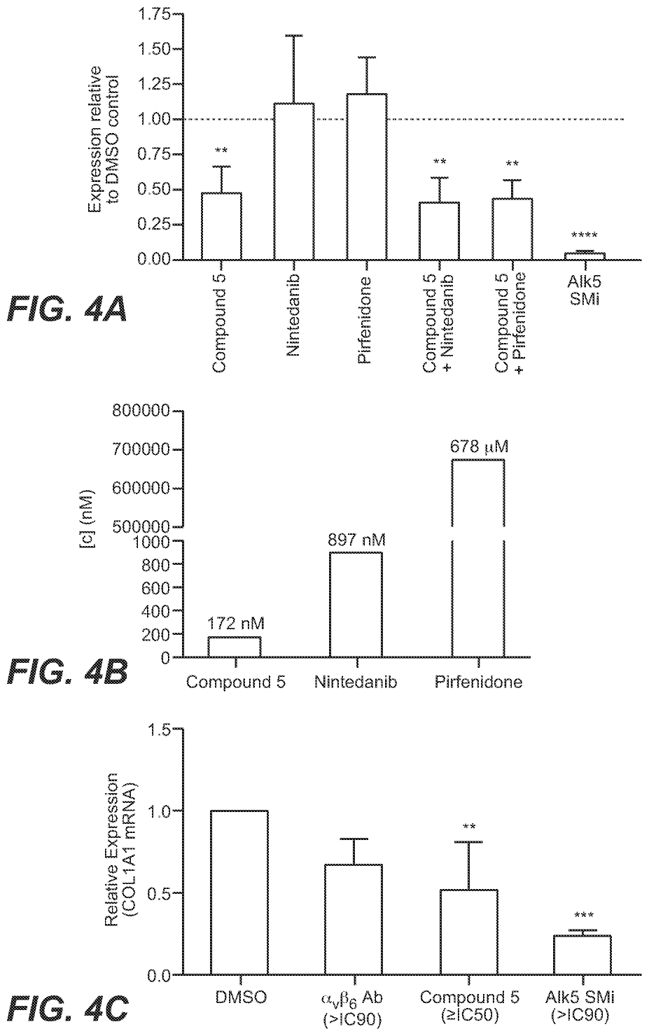

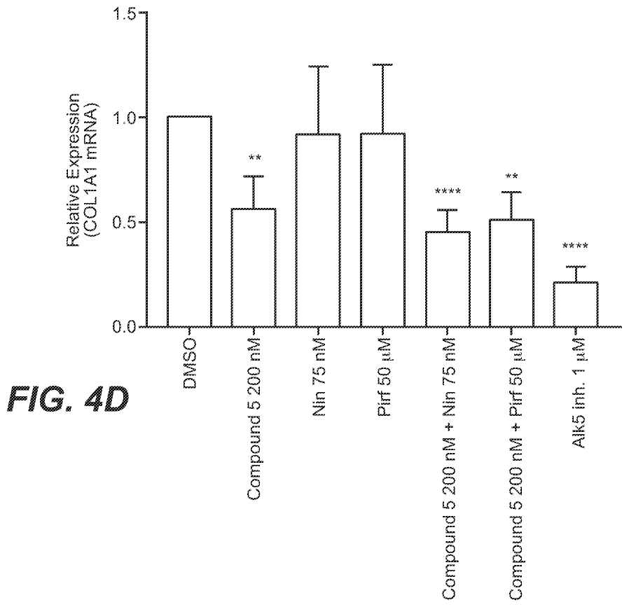

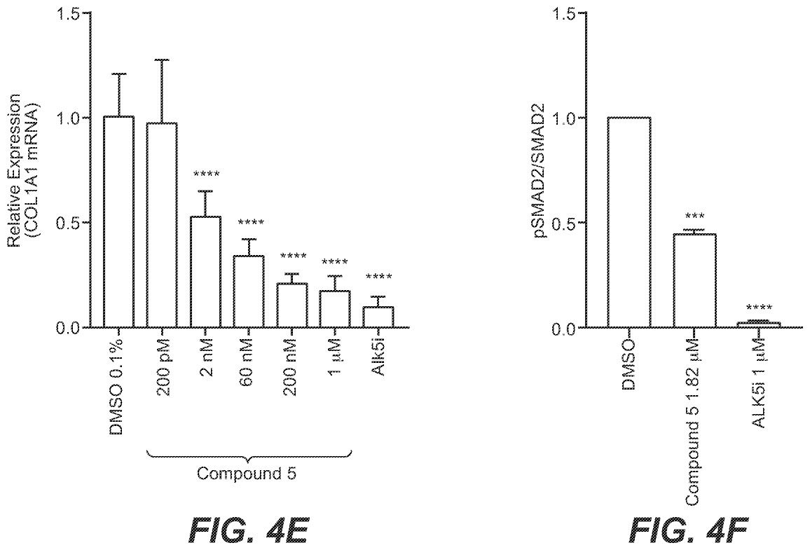

FIG. 4A is a bar graph showing that compared to the DMSO vehicle control slices, both nintedanib and pirfenidone showed a slight increase in lung Col1a1 expression, although the increase was not shown to be statistically significant. By contrast, compound 5 both alone (p<0.01 vs vehicle) and in combination with nintedanib or pirfenidone showed a substantial, statistically significant (p<0.01 vs vehicle) decrease in lung slice Col1a1 expression. Likewise, the small molecule ALK5 inhibitor, used as a positive control representative of total TGF-beta signaling inhibition, showed a substantial, statistically significant (p<0.0001 vs vehicle) decrease in lung Col1a1 expression.

FIG. 4B is a bar graph showing the concentration of compound needed to reduce lung slice Col1a1 expression by 50% compared to DMSO control slices. Data for FIG. 4B was obtained using acute bleomycin injured lung slices prepared as described in Example B3. To match the efficacy of compound 5, nintedanib required a 5.2 fold increase in concentration over compound 5, and pirfenidone required a 3,940-fold increase in concentration over compound 5.

### Example B6-Dual α_{V}β₁/α_{V}β₆ Inhibition Significantly Reduces Collagen Gene Expression in Precision Cut Lung Slices from Human IPF Explants

Explanted lung tissue was obtained from human IPF subjects and inflated with agarose as described in the preceding examples. Biopsy cores were obtained from the agarose-inflated lung tissue. The biopsy cores were subjected to precision slicing to obtain several hundred μm thick. Individual slices were distributed in a multiwell culture plate and cultured for 3 days under control (DMSO) and test compound conditions. The viability of the slices over the course of culturing was confirmed by WST-1 assay of mitochondrial activity.

During the culture period, slices in the control group were treated with DMSO and slices in the test group were treated with a DMSO solution of one of: the selective antibody α_{V}β₆ inhibitor, 3G9, at ≥400 ng/mL; compound 5, at 179 nM; and the small molecule ALK5 inhibitor at 1 μM.

FIG. 4C is a bar graph, normalized to control slices treated with DMSO, showing that all test treatments reduced lung Col1a1 expression. The selective antibody α_{V}β₆ inhibitor, 3G9, slightly reduced lung Col1a1 expression, but was not statistically significant. Compound 5 showed a substantial, statistically significant (p<0.01 vs vehicle) decrease in lung Col1a1 expression, as did the small molecule ALK5 inhibitor (p<0.0001 vs vehicle). Notably, in these human IPF subject samples, compound 5 was much closer in efficacy to the small molecule ALK5 inhibitor than in the murine bleomycin model.

PCLS from 5-7 idopathic pulmonary fibrosis (IPF) lung tissue samples were cultured for seven days with one of: DMSO; Compound 5 at 200 nM; nintedanib at 75 nM; pirfenidone at 50 μm; a combination of Compound 5 at 200 nM and nintedanib at 75 nM; a combination of Compound 5 at 200 nM and pirfenidone at 50 μm; or an Alk5 inhibitor at 1 μm. Compound 5 alone or in combination with nintedanib or pirfenidone reduced COL1A1 expression by 43%, 55%, and 49%, respectively. Nintedanib and pirfenidone treatment alone did not significantly reduce expression of COL1A1. FIG. 4D is a bar graph showing relative expression of COL1A1 in precision cut lung slices (PCLS) from idopathic pulmonary fibrosis (IPF) lung tissue upon exposure to Compound 5, clinical standard of care compounds nintedanib (Nin) and pirfenidone (Pirf), and an ALK5 inhibitor, all versus DMSO control.

PCLS from a single IPF lung tissue sample were cultured for seven days with Compound 5 at concentrations of 200 μM, 2 nM, 60 nM, 200 nM, and 1 μM, along with 0.1% DMSO control and an Alk5 inhibitor at 1 μM. There was a dose dependent reduction in COL1A1 expression with a significant reduction observed≥2 nM (≥47% reduction). FIG. 4E is a bar graph showing a dose dependent reduction of COL1A1 expression in PCLS from human IPF lung tissue upon treatment with concentrations of compound 5 ranging from 200 μM to 1 μM. COL1A1 expression is also graphed for the PCLS in the presence of 0.1% DMSO control, and an Alk5 inhibitor at 1 μM.

PCLS from 3 IPF lung tissues were cultured for seven days with Compound 5. Dual inhibition of α_{V}β₆ and α_{V}β₁ with Compound 5 significantly reduced pSMAD2/SMAD2 ratio, a marker of the canonical TGF-β signaling pathway, in PCLS by approximately 50%. FIG. 4F is a bar graph showing the effect of dual selective α_{V}β₆ and α_{V}β₁ inhibition (Compound 5 at 1.82 μM) on the ratio of pSMAD2/SMAD2 in PCLS from human IPF lung tissue samples. The ratio of pSMAD2/SMAD2 is also graphed for the PCLS in the presence of 0.1% DMSO control, and an Alk5 inhibitor at 1 μM

### Example B7-A Dual α_{V}β₁/α_{V}β₆ Inhibitor Demonstrates Good Oral Bioavailability and Pharmacokinetics in Healthy Human Subjects

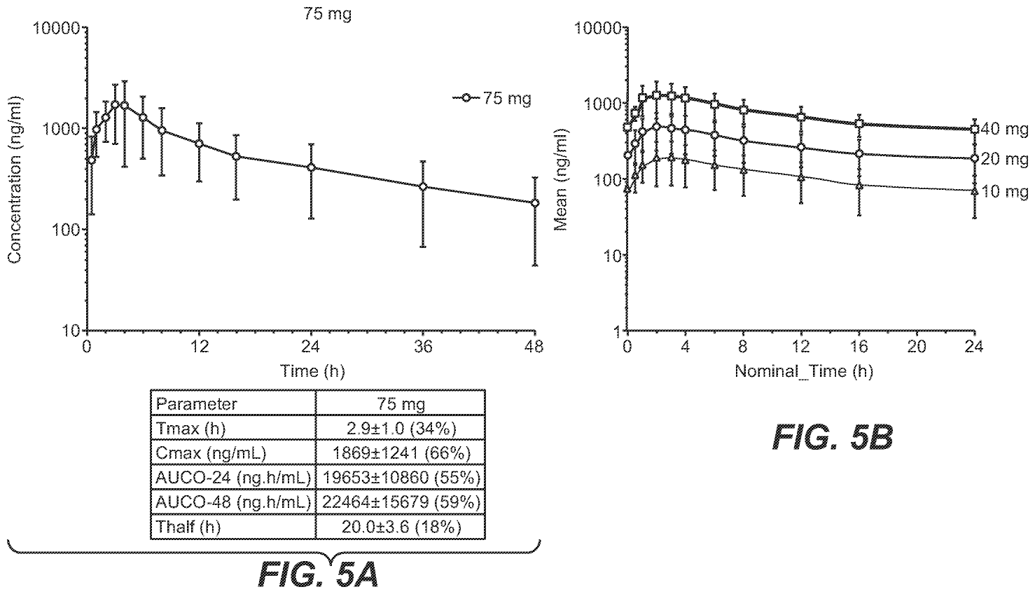

Healthy human subjects (N=85) were selected for single ascending dose (SAD) and multiple ascending dose (MAD) first-in-human studies. A solution for oral administration was prepared, containing 10 mg/mL of compound 5 in a 50:50 mixture of ORA-SWEET® SF (PERRIGO®, Allegan, Michigan) and sterile water for irrigation. Sufficient solution was administered orally to the subjects to provide between 15 mg/dose and 75 mg/dose of compound 5 in the SAD study and between 10 mg/dose and 40 mg/dose of compound 5 in the MAD study. Concentrations of compound 5 were measured in the subjects by obtaining a sample of plasma from each subject at desired intervals, and subjecting the plasma to liquid chromatography-mass spectrometry-mass spectrometry (LC-MS/MS), with quantification using a calibration curve determined from a range of solutions at standardized concentrations. The lower limit of quantitation (LLOQ) of the assay was 1 ng/mL and the calibration curve range was 1 to 500 ng/mL. FIG. 5A shows an example of the SAD study data for administration of 15, 30, 50, and 75 mg of compound 5, and further PK data for 75 mg, which is representative of the results obtained for SAD doses at 15, 30, and 50 mg. FIG. 5B shows the MAD study data for administration of 10, 20, and 40 mg of compound 5. The calculated half life of the compound varied between 18-20 hours, which supports daily administration, such as once-daily administration.

### Example B8-A Dual α_{V}β₁/α_{V}β₆ Inhibitor Demonstrates Reduction of pSMAD2/SMAD2 in BAL from Healthy Human Subjects

In order to evaluate the change of pSMAD2 as a biomarker of TGF-β activity following administration of an integrin inhibitor, and to determine a therapeutically effective dosage and an effective blood plasma Cₘₐₓ of the integrin inhibitor, healthy subjects were administered compound 5, a dual selective α_{V}β₆/α_{V}β₁-integrin inhibitor, and the corresponding Cₘₐₓ levels and decrease in phosphorylation levels and were determined.

Healthy non-smoking adult males without history of lung disease were selected as subjects and were randomized into 4 cohorts. Broncoalevaolar lavage samples were obtained from all subjects 1 day prior to start of treatment. Cohorts 1 and 2 were administered 20 mg of a compound daily, wherein 3 subjects were administered the dual selective α_{V}β₆/α_{V}β₁-integrin inhibitor (compound 5) per every 1 subject receiving a placebo compound. Cohorts 3 and 4 were administered 40 mg of a compound daily, wherein 3 subjects were administered the dual selective α_{V}β₆/α_{V}β₁-integrin inhibitor (compound 5) per every 1 subject receiving a placebo compound. BAL samples and blood samples were taken from all subjects on Day −1 (baseline) and on Day 7 (end of treatment).

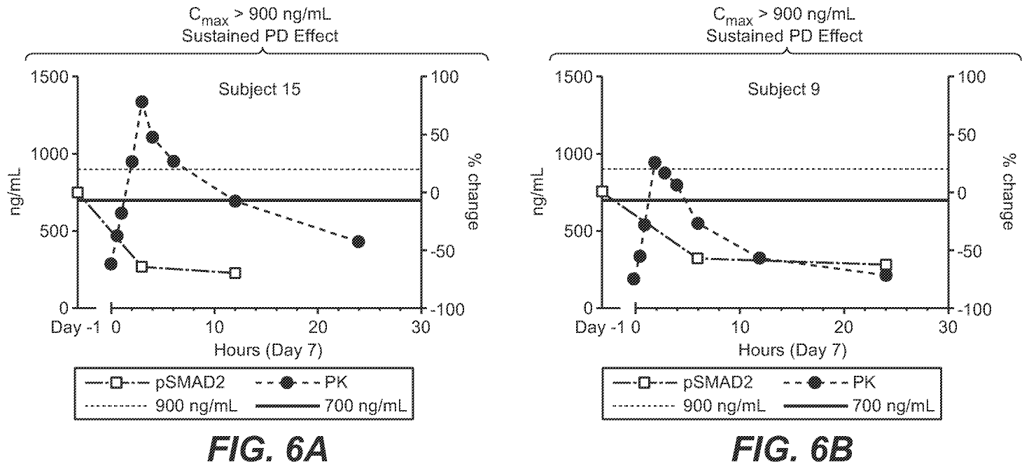

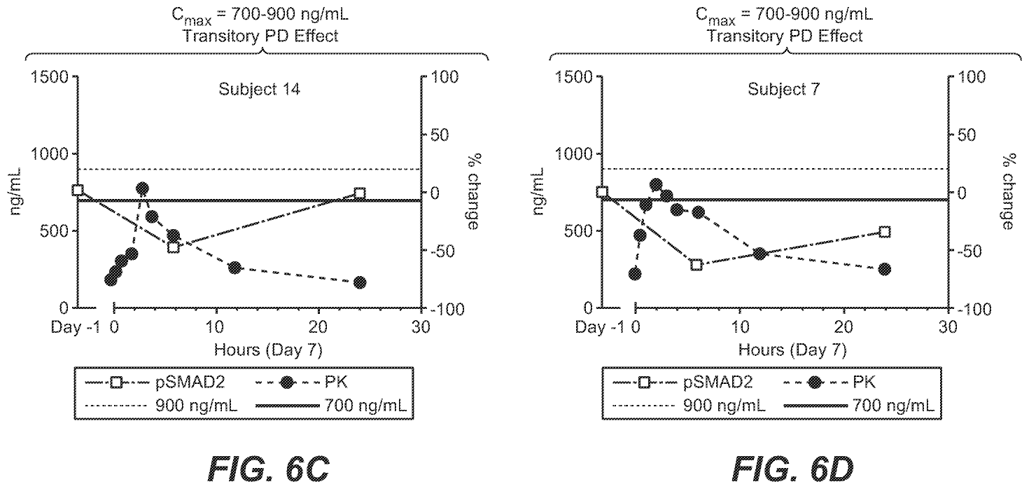

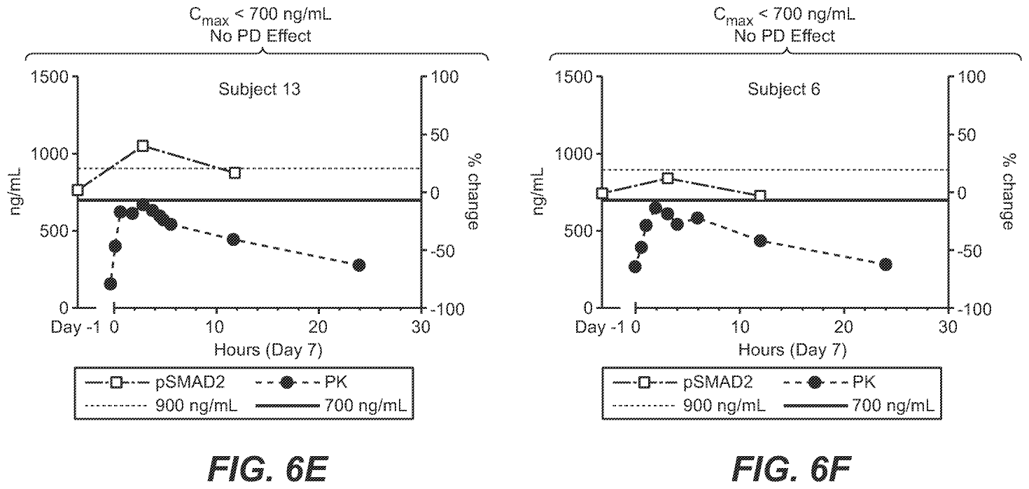

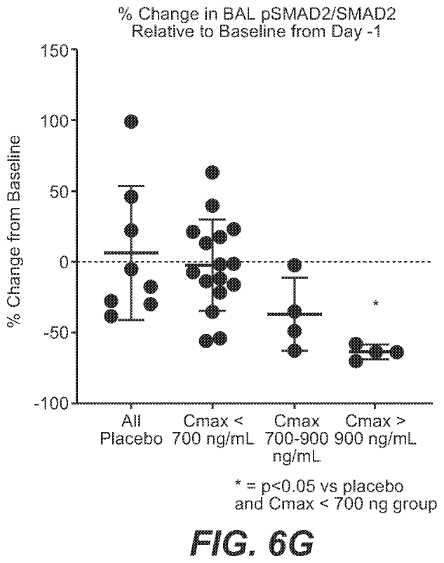

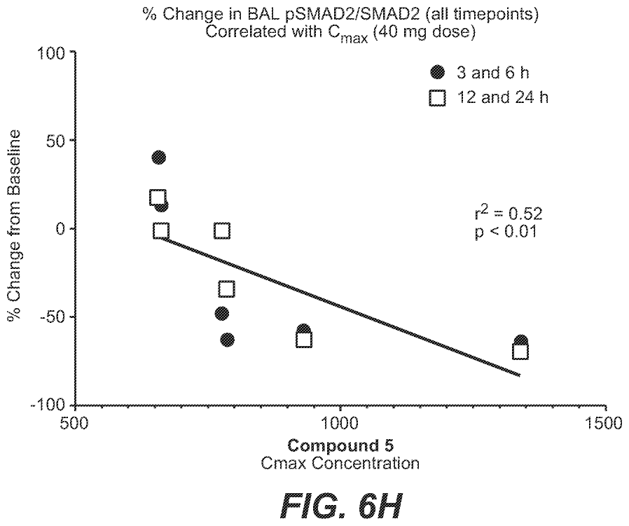

As shown in FIG. 6A, a reduction in pSMAD2:SMAD2 ratio of about 50% or more was achieved in subjects which showed higher blood plasma Cₘₐₓ of the dual selective α_{V}β₆/α_{V}β₁-integrin inhibitor (compound 5) (subjects 15, 9, 14, 7). All subjects with Cₘₐₓ above 700 ng/mL exhibited about 50% or more reduction in pSMAD2:SMAD2 ratio when compared to the placebo group (FIG. 6G) using the dual selective α_{V}β₆/α_{V}3 integrin inhibitor (compound 5). The Cₘₐₓ and pSMAD2:SMAD2 ratio modulations are plotted in FIG. 6H to further illustrate the relationships between dose and pSMAD2 levels. As shown in FIG. 6H, the plasma Cₘₐₓ strongly correlated with reduction of pSMAD2:SMAD2 ratio relative to baseline at 12 h and 24 h post-administration on Day 7.

### DISCUSSION

In human and murine fibrotic lung tissue, α_{V}β₆ (in epithelial cells) and α_{V}β₁ (in fibroblasts) integrin levels are elevated and contribute to the activation of TGF-β. SMAD2/3 phosphorylation in lung tissue and BAL macrophages reflects TGF-β activation and corresponds to fibrogenic activity. SMAD2/3 phosphorylation in healthy lung tissue and BAL macrophages respond to integrin inhibitors reflecting reduced TGF-β activation. Accordingly, SMAD2 phosphorylation in BAL macrophages has been used as described herein to determine dose response and duration of inhibition of integrin inhibitors in clinical studies to establish precise PK/PD models. Dual inhibition of α_{V}β₆ and α_{V}β₁ with compound 5 also significantly reduced SMAD3 phosphorylation and fibrotic collagen deposition in the bleomycin mouse model. Dual inhibition of α_{V}β₆ and α_{V}β₁ with compound 5 significantly reduces collagen gene expression in precision cut lung slices prepared from bleomycin-injured mouse lung and from human IPF subjects. Compound 5 is comparable in antifibrotic activity to pan-α_{V} inhibitors, and may have fewer off-target effects due to selectivity for α_{V}β₆ and α_{V}β₁. Further, dual inhibition of α_{V}β₆ and α_{V}β₁ with compound 5 is more effective than inhibition of either α_{V}β₆ or α_{V}β₁ alone. Finally, compound 5 demonstrated good oral bioavailability and pharmacokinetics in healthy subjects, offering a targeted small molecule approach for blocking TGF-β activity in pulmonary fibrosis.

### Example B9—Target Engagement of Compound 5 to α_{V}β₆ in Participants With IPF Using [¹⁸F]FP-R01-MG-F2 PET/Computerized Tomography (CT) Imaging

Integrin α_{V}β₆ plays a key role in promoting transforming growth factor beta activation in fibrotic diseases and can be imaged via positron emission tomography (PET) with the novel anti-α_{V}β₆ cystine knot peptide (knottin) radiotracer, [¹⁸F]FP-R01-MG-F2 (Kimura, et al., “Evaluation of integrin α_{V}β₆ cystine knot PET tracers to detect cancer and idiopathic pulmonary fibrosis,” Nature Communications (2019) 10:4673; doi:10.1038/s41467-019-11863-w.). The aim of this Example is to assess target engagement of the disclosed compounds to α_{V}β₆ in participants with IPF using [¹⁸F]FP-R01-MG-F2 PET/computerized tomography (CT) imaging.

This Example was conducted as an open-label, single-dose (60 mg, 120 mg, 240 mg or 320 mg) clinical trial evaluating α_{V}β₆ receptor occupancy in the lungs, safety, and pharmacokinetics of Compound 5 in subjects with IPF. Knottin tracer uptake kinetics were compared pre- and post-dose of the disclosed compound, as measured by standardized uptake values (SUVs) and parameters estimated from kinetic modeling in regions-of-interest on dynamic [¹⁸F]FP-R01-MG-F2 PET/CT scans. A two-compartment model (lung and blood) with an image-derived input function was used to fit the measured PET data (See, e.g., Peletier, et al., Impact of protein binding on receptor occupancy: a two-compartment model” J Theor Biol. 2010 Aug. 21; 265(4):657-71. doi: 10.1016/j.jtbi.2010.05.035). Receptor occupancy was estimated from the output of the two-compartment model (V_{T}, the volume of labeled tissue) using standard equations and fitting algorithms, and corrections for VND (non-displaceable tracer binding). For example, in FIG. 8, the data points, shown as circles, are modeled by the following formula:

$${V}_{T , pred} = {V}_{ND} + {V}_{S} ( 1 - \frac{C}{C + {EC}_{50}} )$$

to produce the depicted S curve, wherein V_{T,pred} is the predicted/fit value (S curve) of volume of labeled tissue, V_{ND} is non-displaceable binding, V_{S} is volume of displaceable binding, C is blood concentration of the disclosed compound, and EC50 is the concentration of the disclosed compound that displaces 50% of the labeled knottin tracer.

Five subjects with IPF were enrolled: Subject A received a single dose of 60 mg of the disclosed compound followed by a post-dose scan. Subject B received two doses of the disclosed compound separated by two weeks, 120 mg and 240 mg, each followed by a post-dose scan. Subject C received two doses of the disclosed compound separated by two weeks, 240 mg and 320 mg, each followed by a post-dose scan. Subject D received a single dose of 320 mg of the disclosed compound followed by a post-dose scan. Table B-1 shows the subjects, doses, and various input and measured values for the fit.

TABLE B-1

| | | | | | Corrected For | | |
|---|---|---|---|---|---|---|---|
| | | | | | Non-Displaceable | | |
| | | | Input Values | | Binding (V_{ND} = 0.66) | | |
| Sub- | Dose | Conc¹ | Baseline | | Baseline | | % |
| ject | (mg) | (nM) | Vt | Vt | Vₛ | Vₛ² | DeltaVt³ |
| A₆₀ | 60 | 3.66 | 3.13 | 1.84 | 2.47 | 1.18 | 52.23 |
| B₁₂₀ | 120 | 10 | 3.12 | 1.65 | 2.48 | 0.99 | 59.76 |
| B₂₄₀ | 240 | 58.3 | 3.12 | 0.72 | 2.46 | 0.06 | 97.56 |
| C₂₄₀ | 240 | 103 | 1.76 | 0.98 | 1.1 | 0,32 | 70.91 |
| C₃₂₀ | 320 | 98.5 | 1.76 | 0.85 | 1.1 | 0.19 | 82.73 |
| D₃₂₀ | 320 | 33.6 | 2.06 | 0.75 | 1.4 | 0.09 | 93,57 |

¹Unbound Plasma Concentration
²Baseline Vs = (Baseline Vt) - V_{ND}, Vₛ = Vt − V_{ND}
³% DeltaVT = [(Baseline Vs − Vs)/Baseline Vs] * 100

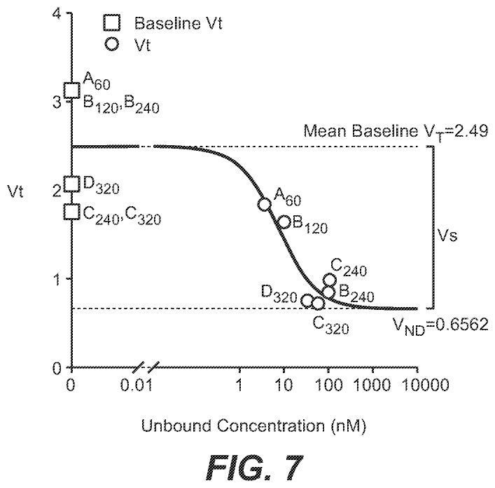

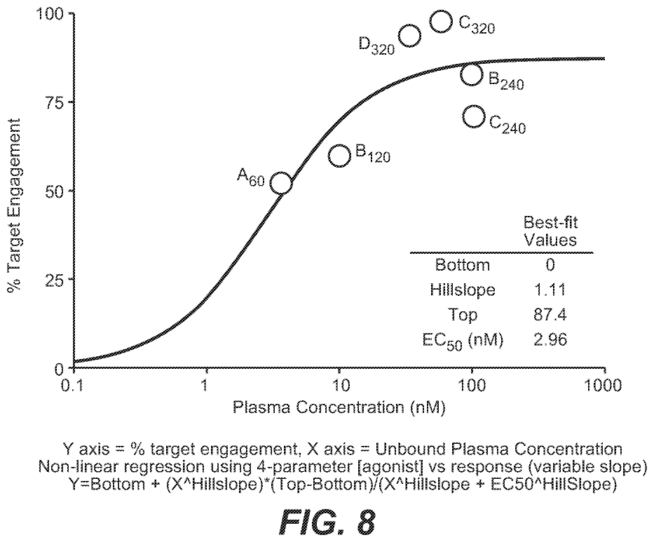

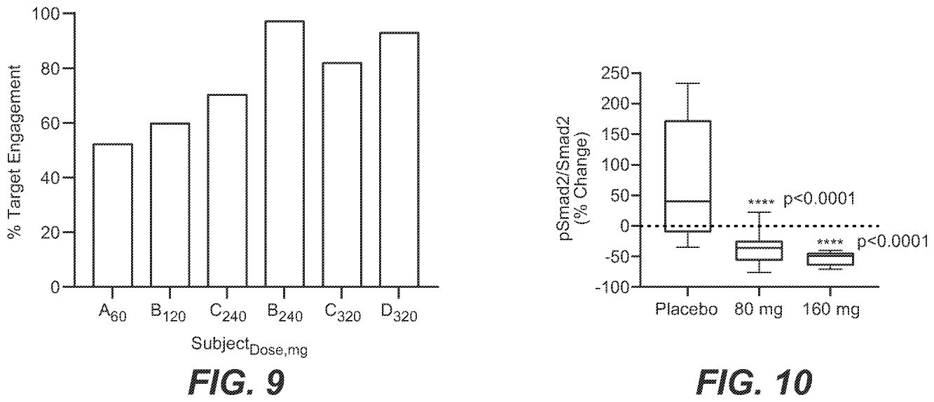

Pre-dose [¹⁸F]FP-R01-MG-F2 PET scans revealed increased α_{V}β₆ expression in the most fibrotic regions of the lungs. When comparing pre- and post-dose PET scans, regions with the highest α_{V}β₆ expression showed the most pronounced reductions in signal, as the disclosed compound displaced the knottin radiotracer. The volume of distribution of the knottin radiotracer in the lungs decreased in a dose-responsive manner, from approximately 50% in the 60 mg dose to greater than 95% in the 240 and 320 mg doses. When calculated based on measured drug exposure at 4 hours, the same pattern was observed with an exposure response saturating at a concentration of about 100 nM, and approaching 100% receptor occupancy. FIG. 7 is a graph of unbound plasma concentration (X-axis) vs Vt (Y-axis) for the baseline Vt at each dose, the measured Vt after each dose, and a fit line. FIG. 8 is a graph of unbound plasma concentration (X-axis) vs % receptor occupancy (Y-axis). FIG. 9 is a bar chart showing % target engagement for each subject and dose.

Single doses of the disclosed compound were associated with decreased knottin radiotracer accumulation in the lungs of participants with IPF. These findings suggest target engagement of the disclosed compound in IPF lungs and that the anti-α_{V}β₆ knottin PET radiotracer may have clinical utility as a predictive and on-treatment biomarker in IPF. Moreover, these results indicate that the effective distribution of disclosed compound throughout the lung tissue and importantly into regions of high α_{V}β₆ expression and high amounts of fibrosis. Receptor occupancy of >95% indicates that nearly full inhibition of TGF-β activation by α_{V}β₆ can be achieved at pharmacologically relevant plasma concentrations and can indicate a significant reduction in TGF-β driven fibrosis in the lung of IPF patients.

### Example B10—the Disclosed Compound is Safe and Tolerated in IPF Subjects

This study design is a Phase 2a, multicenter, 3-part, randomized, double-blind, dose-ranging, placebo-controlled study designed to evaluate the safety, tolerability, and PK of once-daily (QD) treatment with compound 5 in vivo in human participants with idiopathic pulmonary fibrosis (IPF). Each study part will include up to 28-day screening period, a 4-week (Part A) or 12-week (Parts B and C) treatment period, and a 2-week (±3 days) post-treatment follow-up period.

Part A enrollment has been completed, with 54 total participants enrolled. At the time of enrollment, 44 of these 54 participants were undergoing a pre-existing course of therapy using one of the SoC (Standard of Care) compounds (Nintedanib or Pirfenidone). It is anticipated that these participants will continue the SoC therapy throughout this study.

Part B has initiated dosing and Part C will commence following review of the clinical data supporting the evaluation of higher doses. Potential participants who provide written informed consent will be screened for study eligibility up to 28 days before administration of the first dose of compound 5.

In Parts B and C, eligible participants will be randomized on Day 1 (Visit 2). Study treatment will be administered for 12 weeks. Randomization will be stratified by use of standard of care (SoC) IPF therapy (pirfenidone or nintedanib) (SoC use; yes or no).

In Part B, 28 eligible participants will be randomized in a 3:1 ratio (active:placebo). In Part C, 2 additional compound 5 dose groups of 80 mg and 160 mg are planned for evaluation in parallel treatment groups based on the following criteria:
Part B has been completely enrolled (28 participants have randomized.)
Favorable review by the Data Safety Management Board (DSMB) of:
   All available safety and PK data from this study (Parts A and B)
   Safety and PK data from compound 5 in an ongoing Phase 1 study evaluating the safety, tolerability, and pharmacokinetics of compound 5 at multiple doses ranging from 80 to 160 mg in healthy participants

In Part C, approximately 56 eligible participants will be randomized in a 3:3:2 ratio (80 mg compound 5:160 mg compound 5:placebo) and treated for 12 weeks in parallel treatment groups. The total number of participants enrolled in Parts B and C of the study will be approximately 84, with approximately 63 receiving compound 5 and 21 receiving placebo.

Participants who discontinue study drug for safety reasons prior to completion of 12 weeks of treatment will be encouraged to remain in the study to complete all remaining assessments. Where this is not feasible, the subject will be asked to return to the clinic for an Early Termination (ET) visit for follow-up evaluations.

Participant safety will be assessed at predetermined intervals during the study, including evaluation of all safety and PK data to enable initiation of Part C, and as needed. Participants will also be assessed for any adverse events.

### Example B10A—Phase 2a Clinical Trial

The randomized, double-blind, placebo-controlled Phase 2a clinical trial of Compound 5 in patients with idiopathic pulmonary fibrosis (IPF) as described in Example B10 was initiated. The trial met its primary and secondary endpoints demonstrating that Compound 5 was well tolerated over a 12-week treatment period and displayed a favorable pharmacokinetic profile. The trial's exploratory endpoint assessing forced vital capacity (FVC), showed a dose-dependent treatment effect on FVC versus placebo at 12 weeks in Compound 5 treated patients. A dose-dependent reduction was observed in the proportion of patients experiencing a FVCpp decline of ≥10%.

Compound 5 was evaluated at once-daily doses of 40 mg, 80 mg, 160 mg or placebo for 12 weeks in 90 patients with IPF. 67 patients were enrolled in the active arms and 23 patients were enrolled in the placebo arm. Approximately 80% of the enrolled patients were on standard of care and were equally distributed between nintedanib and pirfenidone.

At all three doses tested, Compound 5 was well tolerated. Of the 67 patients treated with Compound 5, 65 (97%) completed 12 weeks of treatment with no discontinuations due to adverse events. No treatment related deaths or drug related serious adverse events (SAE) were reported. Most treatment emergent adverse events (TEAE) were mild or moderate in severity.

Compound 5 exhibited generally dose proportional increases in plasma concentrations, consistent with prior studies.

The exploratory endpoints of the trial measured changes in forced vital capacity (FVC), HRCT-based Quantitative Lung Fibrosis score (QLF), and selected biomarkers over 3 months of treatment.

A treatment effect was observed in all Compound 5 dose groups with and without standard of care therapy. A pooled analysis of Compound 5 treated patients showed an approximately 80% reduction in FVC decline over 12 weeks versus placebo (−15.1 mL for Compound 5 pooled groups versus −74.1 mL for placebo). The 40 mg and 160 mg dose groups demonstrated 38% (−46.1 mL) and 66% (−25.1 mL) reductions in FVC decline, respectively, relative to placebo. Importantly, for the 80 mg treatment group, a +24.6 mL increase in FVC was observed relative to baseline.

At 12 weeks, the proportion of patients who experienced a >2% increase in QLF was lowest in the 80 mg group (11%). The proportion of patients who remained stable (−2 to 2% change) or experienced a decrease in QLF (>2% change) were similar in the 160 mg group (46.6% and 26.7%, respectively) and the placebo (47.1% and 23.5%, respectively), where the approximately 80% of patients received SoC. A treatment effect of Compound 5 is suggested with a greater proportion of patients with a decrease or stable QLF score compared to placebo group. Changes in QLF (%) correlate with changes in FVC (mL) and FVCpp.

A decline of ≥10% in predicted FVC (FVCpp) at 12 weeks is associated with an increased risk of death in IPF patients over a two-year period (Paterniti M O, et al Ann Am Thorac Soc. 2017 September; 14(9):1395-1402). The proportion of patients that experienced a ≥10% decline in FVCpp were 8.7% in the 80 mg group and 4.5% in the 160 mg group versus 17.4% in the placebo group. The 40 mg group experienced a 18.2% decline relative to placebo. The dose-dependent decrease in the proportion of patients with FVCpp decline of ≥10% suggests a potentially disease-modifying effect of Compound 5.

FIG. 10 shows dose dependent effects and TGF-β suppression of Compound 5. Alveolar pSmad2/Smad2 percentage change from baseline at 24 h (Part 1: 80 mg and 160 mg). Percent change pSMAD2/Smad2 was statistically significant at both doses of Compound 5 vs. placebo (p<0.0001). BAL—bronchoalveolar lavage; pSmad2/Smad2—ratio of phosphorylated Smad2 to total Smad2; QD—once daily.

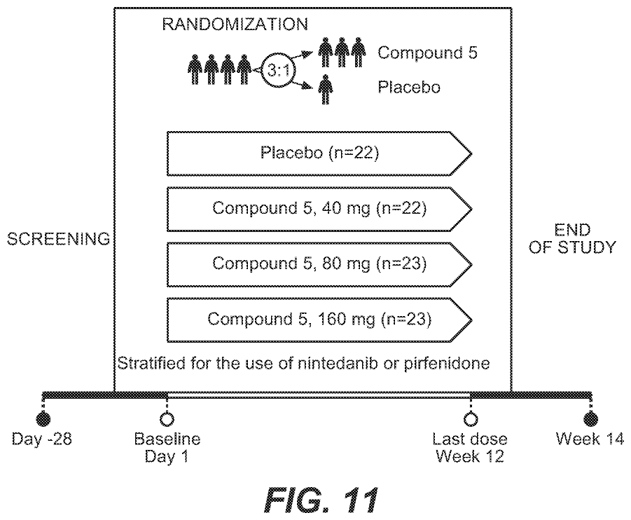

FIG. 11 describes the study design and objectives. Primary and secondary endpoints are safety, tolerability and PK. Exploratory endpoints are 1) change in FVC over 12 weeeks, 2) high resolution CT-based quantitative lung fibrosis (QLF) imaging, 3) patient reported outcome (PRO): VAS-cough severity, and 4) effect on selected biomarkers.

Summary of results of the Compound 5 study:
Compound 5 safe and well tolerated over 12 weeks of treatment
   Most TEAEs were mild or moderate in severity
   No premature discontinuations due to AEs
   No deaths or drug-related SAEs
Compound 5-treated patients experienced an 80% reduction in FVC decline over 12 weeks (−15.1 mL, pooled active groups) compared to placebo (−74.1 mL)
   Compound 5 treatment effect was evident with and without use of standard-of-care agents
   An improvement in FVC (+24.6 mL) was observed in Compound 5, 80 mg dose cohort
   Dose-dependent reduction in proportion of patients with FVCpp decline of ≥10%, a well-established predictor of death and disease progression in IPF
Other exploratory endpoints
   Compound 5 decreased Serum biomarkers of collagen synthesis of PROC3 and 6 relative to placebo

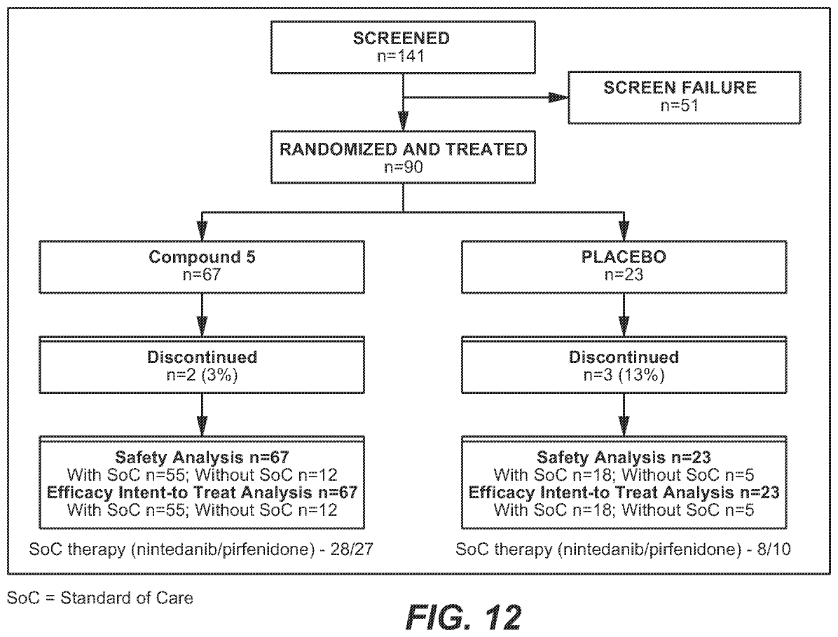

FIG. 12 describes study participant disposition.

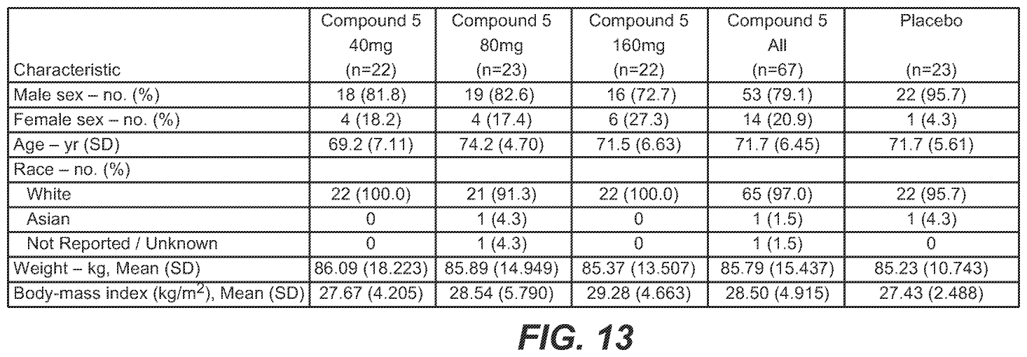

FIG. 13 describes baseline demographics of the study participants. SD=Standard deviation; BMI=Body Mass Index; FVC=Forced Vital Capacity; DLCO=Diffusing capacity for carbon monoxide; SD=standard deviation. Duration since diagnosis at screening is calculated from the first reported date for preferred terms of Idiopathic Pulmonary Fibrosis, Pulmonary Fibrosis or Interstitial Lung Disease. Percentages are based on the number of participants in the Safety Population by treatment group. GAP Stage I=GAP Index 0-3; GAP Stage II=GAP Index 4-5; GAP Stage III=GAP Index 6-8. GAP Index score (0-8) derived from Gender, Age, FVC, % Predicted and DLCO, % Predicted.

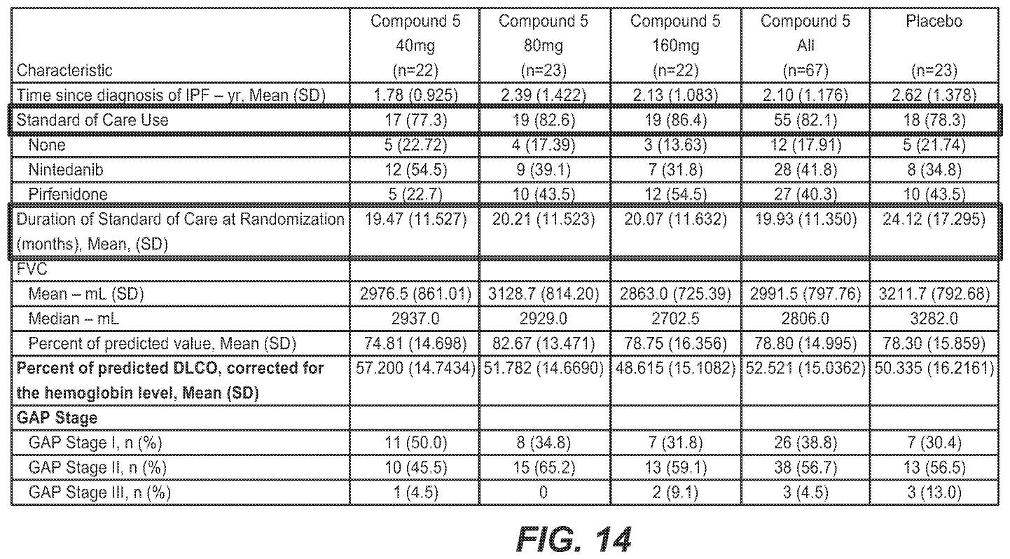

FIG. 14 describes baseline disease characteristics of the study participants. SD=Standard deviation; BMI=Body Mass Index; FVC=Forced Vital Capacity; DLCO=Diffusing capacity for carbon monoxide; SD=standard deviation. GAP Stage I=GAP Index 0-3; GAP Stage II=GAP Index 4-5; GAP Stage III=GAP Index 6-8. Duration since diagnosis at screening is calculated from the first reported date for preferred terms of Idiopathic Pulmonary Fibrosis, Pulmonary Fibrosis or Interstitial Lung Disease. GAP Index score (0-8) derived from Gender, Age, FVC, % Predicted and DLCO, % Predicted. Percentages are based on the number of participants in the Safety Population by treatment group.

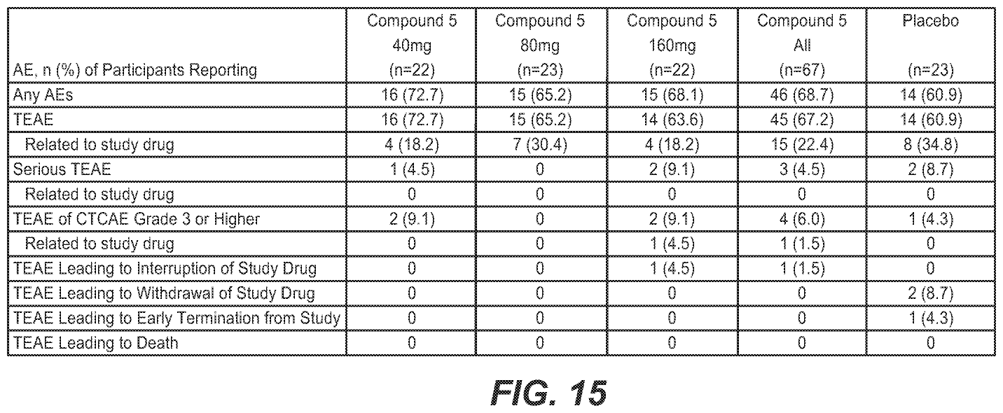

FIG. 15 summarizes overall safety of the study. AE=Adverse Event; TEAE=Treatment Emergent Adverse Event; SAE=Serious Adverse Events. Adverse events coded using MedDRA v. 24.0. TEAE is defined as any AE starting (or worsening) on or after the date of first dose.

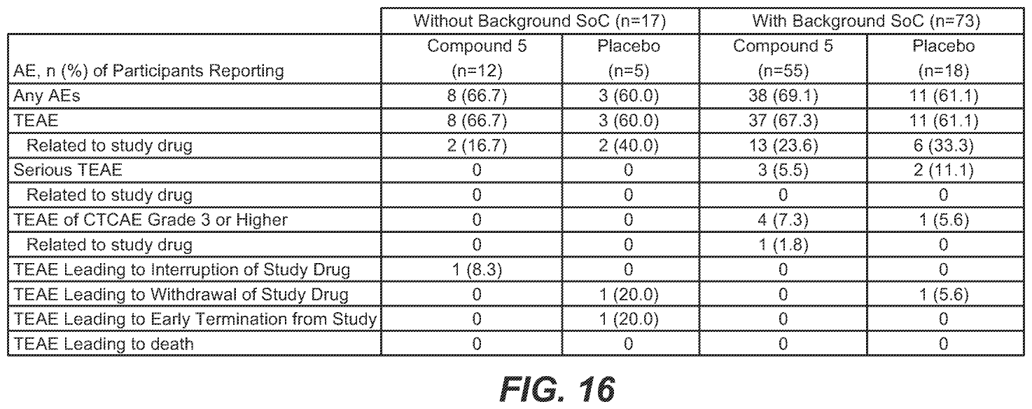

FIG. 16 summarizes overall safety of the study with and without background Standard of Care (SoC) in pooled compound 5 groups. TEAE=Treatment Emergent Adverse Event; SAE=Serious Adverse Events. Adverse events coded using MedDRA version 24.0. TEAE is defined as any AE starting (or worsening) on or after the date of first dose. SOC=standard of care, nintedanib or pirfenidone.

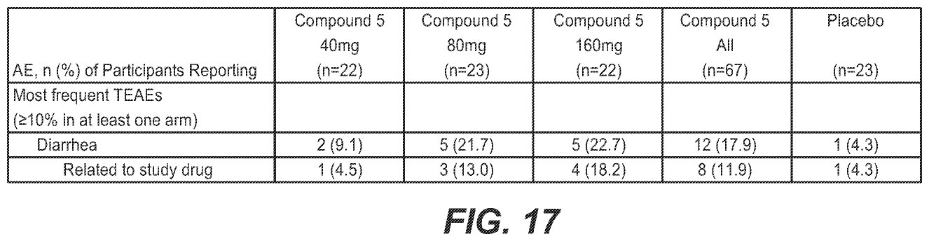

FIG. 17 describes the most frequent treatment emergent adverse events (TEAEs)—any causality. All TEAEs of diarrhea occurred in participants on SoC. 12 of 13 participants with diarrhea were taking nintedanib. All but one event were mild to moderate in severity. TEAE=Treatment Emergent Adverse Event; SAE=Serious Adverse Events. Adverse events coded using MedDRA version 24.0. TEAE is defined as any AE starting (or worsening) on or after the date of first dose.

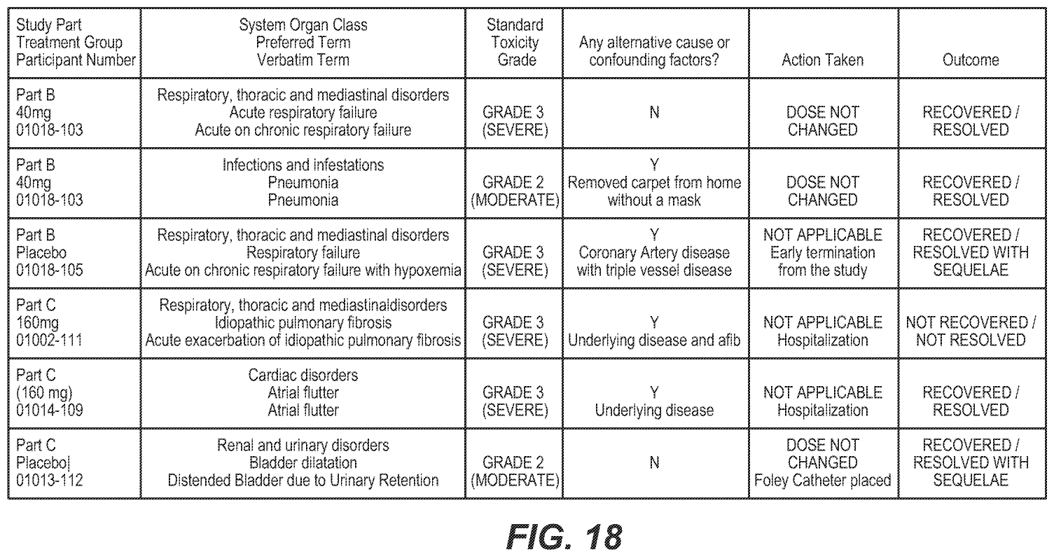

FIG. 18 illustrates that no treatment-emergent serious adverse events (SAEs) were observed with Compound 5. TEAE=Treatment Emergent Adverse Event; Adverse events coded using MedDRA version 24.0. TEAE is defined as any AE starting (or worsening) on or after the date of first dose.

Overall Summary of Safety Evaluation. Compound 5 was well tolerated with no dose relationship or adverse events, no treatment related SAEs or deaths, no participants discontinued Compound 5 due to TEAE, most frequent TEAE seen was diarrhea but only seen in patients on standard of care.

Overall Summary of Safety Pharmacokinetics. Based on sparce sampling, overall Compound 5 pharmacokinetics and % unbound in IPF consistent with that of previous studies, concentrations in IPF participants increased approximately proportionally with dose, overall % unbound was ˜0.3 to 0.5%, full PK curve will be predicted using population PK model to project AUCO₂₄ and Cₘₐₓ.

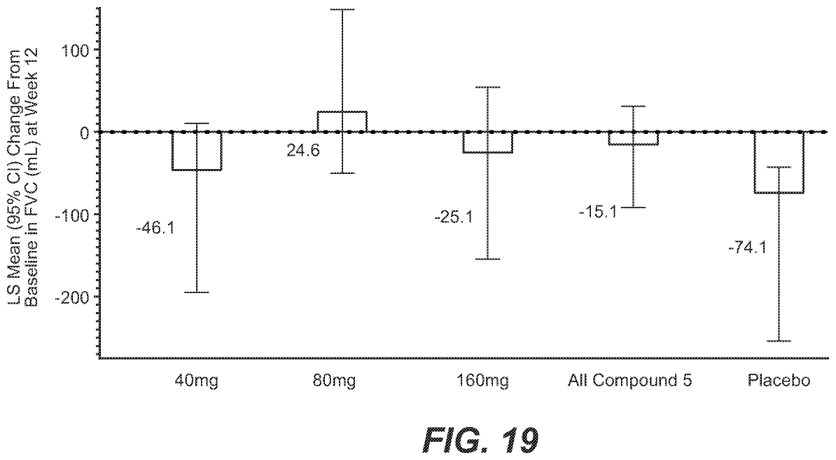

FIG. 19 illustrates the change in FVC (forced vital capacity) from baseline to Week 12, MMRM Analysis—ITT Population. Change from baseline analyzed using a mixed model for repeated measures with terms for treatment group, SOC (Y/N), visit, baseline value, and treatment-by-visit interaction. An unstructured covariance (UN) structure was used.

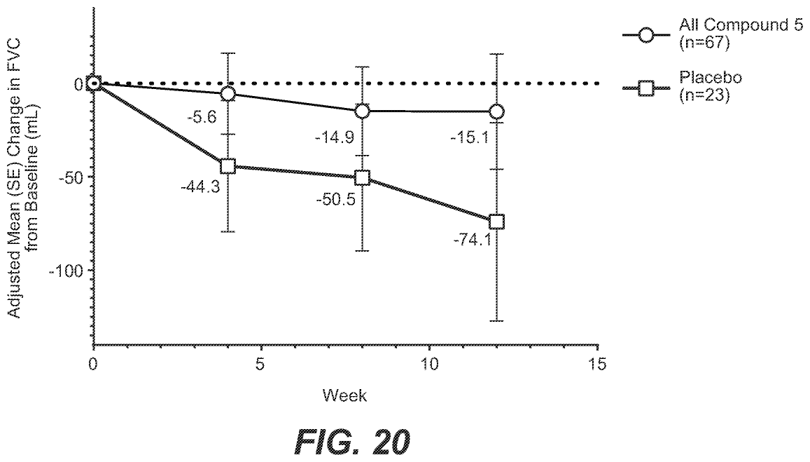

FIG. 20 illustrates the change in FVC over time in pooled Compound 5 groups. MMRM Analysis—ITT Population. FVC=Forced Vital Capacity. MMRM=Mixed Model Repeat Measures.

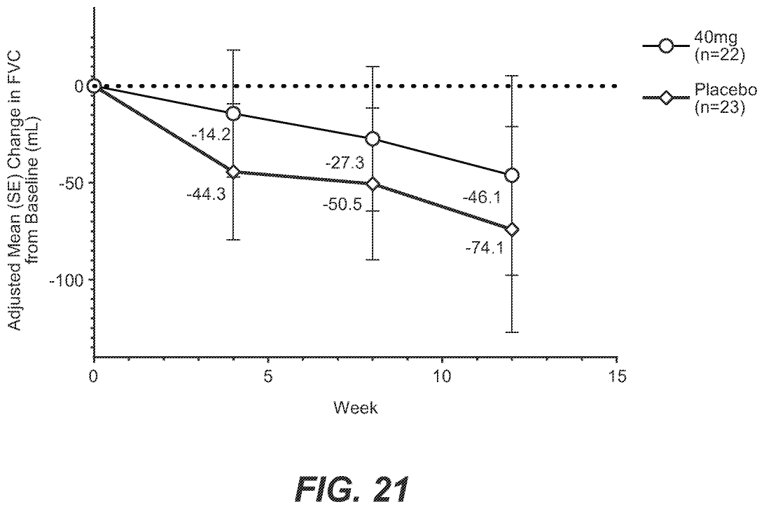

FIG. 21 illustrates the change in FVC over time in the 40 mg Compound 5 group. MMRM Analysis—ITT Population. FVC=Forced Vital Capacity. MMRM=Mixed Model Repeat Measures.

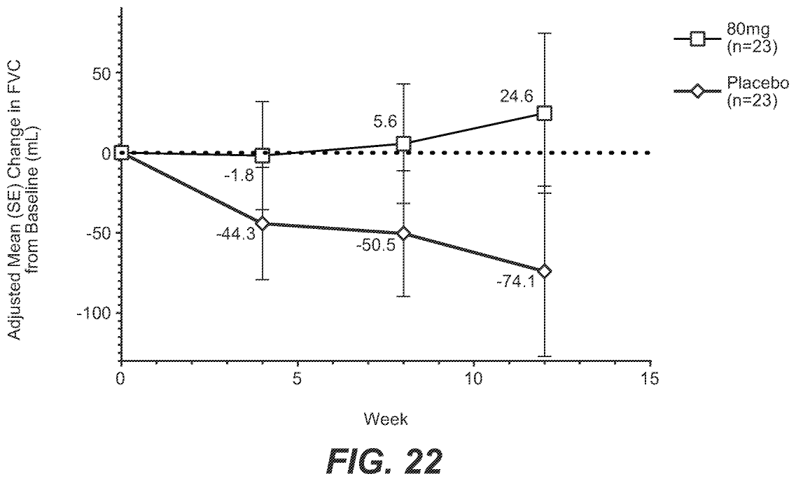

FIG. 22 illustrates the change in FVC over time in the 80 mg Compound 5 group. MMRM Analysis—ITT Population. FVC=Forced Vital Capacity. MMRM=Mixed Model Repeat Measures.

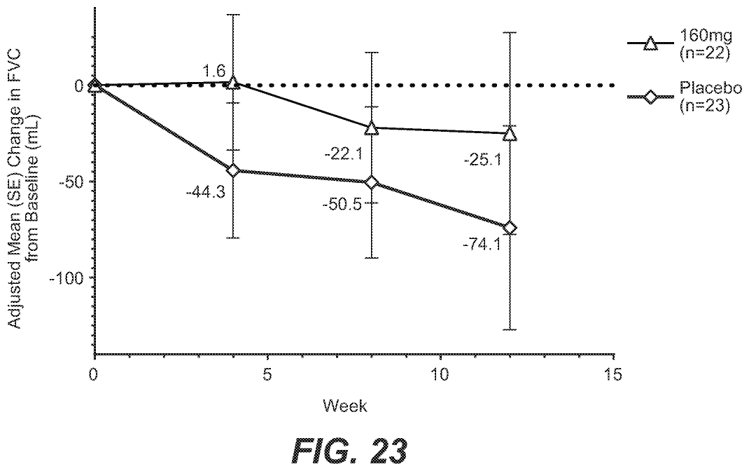

FIG. 23 illustrates the change in FVC over time in the 160 mg Compound 5 group. MMRM Analysis—ITT Population. FVC=Forced Vital Capacity. MMRM=Mixed Model Repeat Measures.

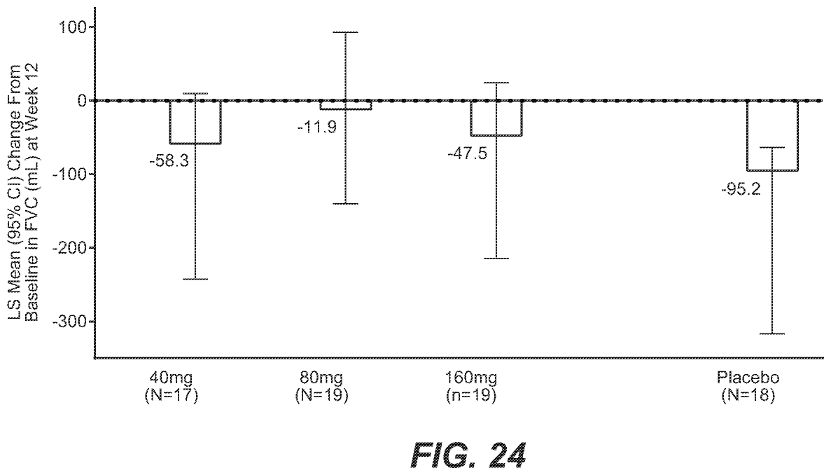

FIG. 24 illustrates the change in FVC from baseline to Week 12 in the subgroup on Standard of Care. MMRM Analysis—ITT Population. Change from baseline analyzed using a mixed model for repeated measures with terms for treatment group, SOC (Y/N), visit, baseline value, and treatment-by-visit interaction. An unstructured covariance (UN) structure was used. FVC=Forced Vital Capacity.

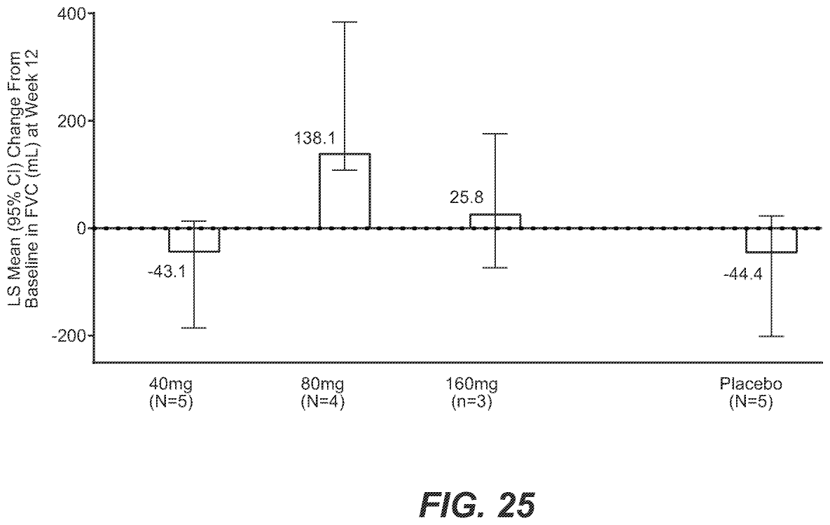

FIG. 25 illustrates the change in FVC from baseline to Week 12 in the subgroup not on Standard of Care. MMRM Analysis—ITT Population. Change from baseline analyzed using a mixed model for repeated measures with terms for treatment group, SOC (Y/N), visit, baseline value, and treatment-by-visit interaction. An unstructured covariance (UN) structure was used. FVC=Forced Vital Capacity.

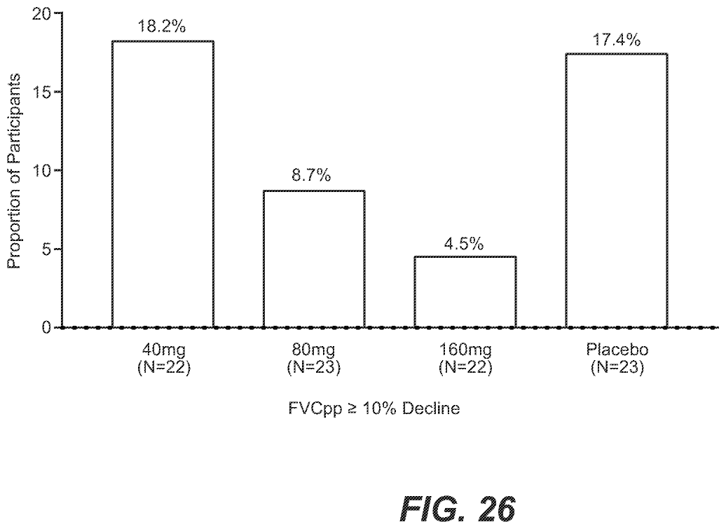

FIG. 26 illustrates the proportion of participants with forced vital capacity-% predicted (FVCpp) decline greater than or equal to 10%. ITT Population. FVCpp=Forced Vital Capacity (% Predicted). FVCpp≥10%: strong predictor of disease progression and mortality (Ann Am Thorac Soc. 2017 September; 14(9):1395-1402, herein incorporated by reference in its entirety).

Overall summary of spirometry evaluation: Compound 5-treated participants experienced a benefit in FVC change from Baseline to Week 123 (−15.1 mL for pooled compound 5 group) compoared to those on placebo (−74.1 mL) (MMRM analysis ITT population). Compound 5 treatment effect was evident with and without use of standard of care. Compound 5 80 mg dose demonstrated an improvement in FVC (+24.6 mL). Dose dependent reduction in proportion of participants with FVCpp decline of ≥10%.

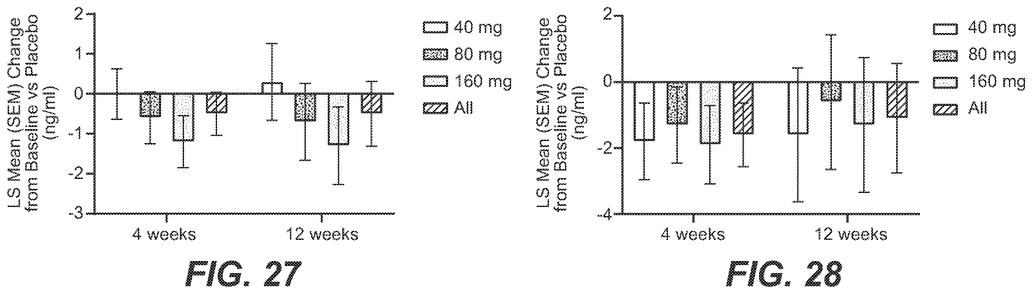

FIG. 27 (PRO—C3 Type III collagen synthesis neoepitope) and FIG. 28 (PRO—C6 TypeVI collagen synthesis neoepitope) compare serum biomarkers of collagen synthesis in Compound 5 groups versus placebo groups. Compound 5 decreased serum biomarkers of collagen synthesis relative to placebo. PRO—C3 and PROC6 (serum biomarkers of type III and VI collagen synthesis, respectively) have previously been shown to be elevated in patients with IPF and associated with progressive disease. Change from baseline of serum PRO—C3 and PRO—C6 levels were reduced in participants receiving Compound 5 vs. placebo (not significant). LS=Least Squares, SEM=Standard Error of Mean.

Study conclusions and next steps. The data from the INTEGRIS-IPF trial showed a favorable safety and tolerability profile and a treatment effect on FVC, the current registrational endpoint in IPF. The treatment effect was also observed on top of standard of care therapy. Interim data from the 320 mg cohort of the trial is anticipated in early 2023.

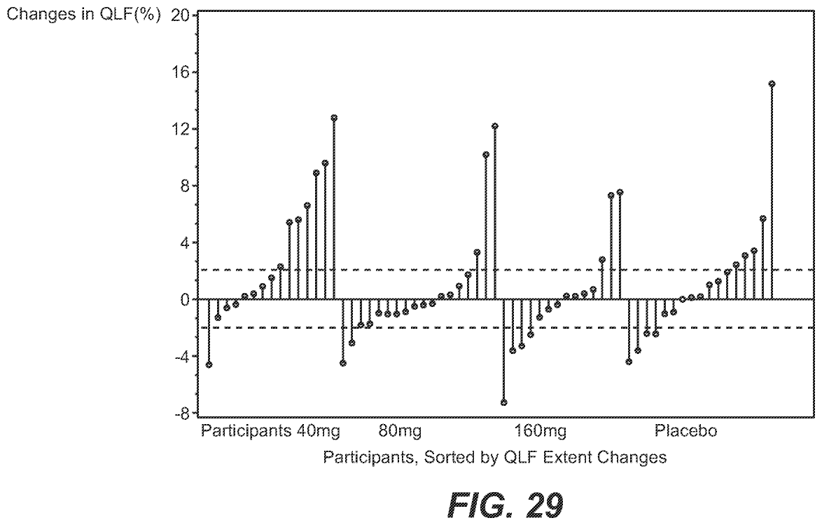

FIG. 29 illustrates the Mean Percent Change in Quantitative Lung Fibrosis (the extent from baseline to week 12 in the CT Protocol Population), using High-Resolution Computed Tomography (HRCT) based Quantitative Lung Fibrosis (QLF) imaging. Participants sorted by QLF extent changes are shown in Table B-2.

TABLE B-2
Participants sorted by QLF extent changes

| | 40 mg | 80 mg | 160 mg | Placebo |
|---|---|---|---|---|
| | (N = 15) | (N = 18) | (N = 14) | (N = 17) |
| Improvers < | 1 (6.6%) | 2 (11.1%) | 4 (28.6%) | 4 (23.5%) |
| −2%, N (%) | | | | |
| Stable [−2, 2%), | 7 (46.7%) | 13 (72.2%) | 7 (50.0%) | 8 (47.1%) |
| N (%) | | | | |
| Worse > 2%, | 7 (46.7%) | 3 (16.7%) | 3 (21.4%) | 5 (29.4%) |
| N (%) | | | | |
| Mean | 3.15% | 0.70% | 0.00% | 1.15% |
| Difference (SD) | (4.80) | (4.19) | (3.96) | (4.47) |
| Median | 1.50% | −0.45% | −0.10% | 0.20% |

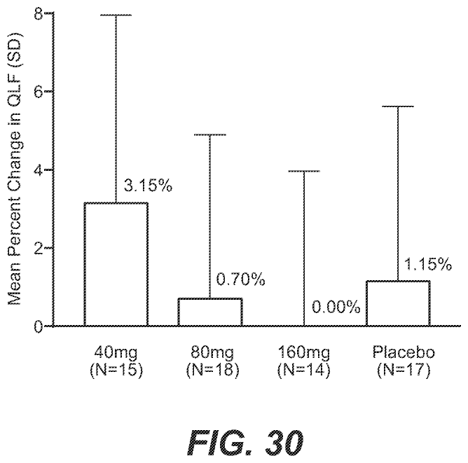

FIG. 30 illustrates the Mean Percent Change in Quantitative Lung Fibrosis (the extent from baseline to week 12) in the CT Protocol Population within Screening Window, using High-Resolution Computed Tomography (HRCT) based Quantitative Lung Fibrosis (QLF) imaging.

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A method for determining a therapeutic dose of a compound, or a pharmaceutically acceptable salt thereof, for treating a condition mediated by at least one integrin, comprising:
(a) administering the compound, or a pharmaceutically acceptable salt thereof, to a subject in need of therapy for the condition, wherein the subject expresses the at least one integrin and the compound, or pharmaceutically acceptable salt thereof, binds to the at least one integrin;
(b) measuring the percent occupancy of the compound in the at least one integrin in the tissue of the subject; and
(c) determining the amount of the compound, or a pharmaceutically acceptable salt thereof, that is effective to achieve a predetermined percent occupancy of the at least one integrin, wherein the amount of compound or pharmaceutically acceptable salt thereof that is required to achieve the predetermined percent occupancy is the therapeutic dose of the compound or a pharmaceutically acceptable salt thereof.

2. The method of claim 1, wherein (a) and (b) are performed on a first subject and one or more additional subjects, wherein the first and the one or more additional subjects have the same condition, and wherein the first subject is administered a first amount of the compound or a pharmaceutically acceptable salt thereof and the one or more additional subjects are each administered an amount of the compound or a pharmaceutically acceptable salt thereof that is different than the amount given to the first subject.

3. The method of claims 1 or 2, wherein the subject has a tissue involved in the condition that expresses the at least one integrin, and wherein the predetermined percent occupancy of the at least one integrin is in the tissue of the subject.

4. The method of any one of claims 1-3, wherein the compound, or a pharmaceutically acceptable salt thereof, is administered to the subject in a single dose.

5. The method of any one of claims 1-3, wherein the compound, or a pharmaceutically acceptable salt thereof, is administered to the subject in two or more doses.

6. The method of claim 5, wherein the first and subsequent doses are administered up to two or three weeks apart.

7. The method of any one of claims 1-6, wherein percent occupancy is measured using data obtained from a PET/CT scan of the subject after administration of the compound, or a pharmaceutically acceptable salt thereof.

8. A method for determining the therapeutically effective percent occupancy of an integrin of a compound that binds to the integrin, or a pharmaceutically acceptable salt thereof, the method comprising:
(a) administering the compound, or a pharmaceutically acceptable salt thereof, to a subject having a condition mediated by at least one integrin and the subject expresses the at least one integrin;
(b) determining the percent occupancy of the compound in at least one integrin in the subject; and
(c) measuring in the subject one or more parameters associated with the condition before and after administration of the compound, or a pharmaceutically acceptable salt thereof,
wherein a beneficial effect on one or more parameters associated with the condition measured after administration of the compound, or pharmaceutically acceptable salt thereof, compared to measurement of the same parameter in the subject before administration of the compound, or a pharmaceutically acceptable salt thereof, indicates a therapeutically effective percent occupancy of the integrin.

9. The method of claim 8, wherein the subject has a condition mediated by at least one integrin and a tissue that expresses the at least one integrin, and wherein percent occupancy of the compound in the at least one integrin in the tissue of the subject is determined.

10. A method of therapy for a condition mediated by at least one integrin, comprising:
providing a subject in need of therapy, the subject comprising: the condition mediated by the at least one integrin, and a tissue involved in the condition that expresses the at least one integrin; and
modulating the at least one integrin in the tissue in the subject effective to treat the condition, comprising administering to the subject at least one compound that binds to a receptor of the at least one integrin, the at least one compound being administered to the subject in an amount effective to achieve a percent occupancy of the receptor of the at least one integrin in the tissue of the subject of at least about 50%.

11. The method of any one of claims 1-10, wherein the percent occupancy corresponds to an anti-fibrotic effect.

12. The method of claim 11, wherein the antifibrotic effect comprises a reduction in SMAD phosphorylation.

13. The method of claims 1 or 2, wherein the percent occupancy of the receptor of the at least one integrin in the tissue of the subject of at least about 50% over a period of at least about one of, or a range between about any two of: 2, 3, 4, 6, 8, 12, 24, 48, 72, 168, 336, or 672 hours.

14. The method of any one of claims 1-13, wherein the at least one compound that binds to a receptor of the at least one integrin is administered once daily.

15. The method of any one of claims 1-13, wherein the at least one compound that binds to a receptor of the at least one integrin is administered twice daily.

16. The method of any one of claims 1-13, wherein the compound that binds to the at least one integrin is administered three times daily.

17. The method of any one of claims 1-15, wherein the percent occupancy is measured by PET/CT.

18. The method of claim 17, wherein the compound displaces a radiolabeled competitive binding agent.

19. The method of claim 18, wherein the compound displaces a knottin radiotracer.

20. The method of claim 19, wherein the knottin radiotracer is 18-F radiolabeled.

21. The method of claim 20, wherein the knottin radiotracer is [¹⁸F]FP-R01-MG-F2.

22. The method of any one of claims 1-21, the at least one compound being administered to the subject in an amount effective to achieve a percent occupancy of the at least one integrin in the tissue of the subject of about one of, or a range between about any two of: 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%.

23. The method of any one of claims 1-22, wherein the compound is orally administered to the subject.

24. The method of any one of claims 1-23, wherein the percent occupancy of the receptor in the tissue of the subject is achieved using a single dose of the compound.

25. The method of any one of claims 1-24, wherein the compound inhibits the at least one integrin in the subject effective to treat the condition.

26. The method of any one of claims 1-25, wherein the tissue has at least one elevated level selected from the group consisting of:
activity and/or expression of the at least one integrin;
a pSMAD/SMAD value;
new collagen formation or accumulation;
total collagen; and
Type I Collagen gene Col1a1 expression;
wherein the level is elevated compared to a healthy state of the tissue.

27. The method of claim 26, wherein the elevated pSMAD/SMAD value is at least one of an elevated pSMAD2/SMAD2 value or an elevated pSMAD3/SMAD3 value.

28. The method of any one of claims 1-27, wherein the tissue is selected from at least one member of the group consisting of: lung tissue, liver tissue, skin tissue, cardiac tissue, kidney tissue, gastrointestinal tissue, gall bladder tissue, and bile duct tissue.

29. The method of any one of claims 1-28, wherein the at least one integrin comprises an α_{V} subunit.

30. The method of any one of claims 1-29, wherein the at least one integrin comprises a β₁ or a β₆ subunit.

31. The method of any one of claims 1-30, wherein the at least one integrin comprises at least one of: α_{V}β₁ integrin and α_{V}β₆ integrin.

32. The method of any one of claims 1-31, wherein the at least one integrin comprises α_{V}β₁ integrin and α_{V}β₆ integrin.

33. The method of any one of claims 1-32, wherein the condition mediated by the at least one integrin is selected from the group consisting of: a fibrotic disease and psoriasis.

34. The method of any one of claims 1-33, wherein the condition mediated by the at least one integrin is a fibrotic disease selected from the group consisting of: idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, scleroderma, diabetic nephropathy, diabetic kidney disease, focal segmental glomerulosclerosis, chronic kidney disease, and Crohn's Disease.

35. The method of claim 34, wherein the compound inhibits the activity of one or both of α_{V}β₁ integrin and α_{V}β₆ integrin in the subject, thereby treating the fibrotic disease in the subject.

36. The method of any one of claims 1-35, wherein the condition mediated by the at least one integrin is NASH, and the compound inhibits the activity of at least α_{V}β₁ integrin in the subject effective to treat the subject for NASH.

37. The method of any one of claims 1-36, wherein the condition mediated by the at least one integrin is IPF, and the compound inhibits the activity of at least α_{V}β₆ integrin in the subject effective to treat the subject for IPF.

38. The method of claim 37, wherein the compound inhibits the activity of α_{V}β₁ integrin and α_{V}β₆ integrin in the subject effective to treat the subject for IPF.

39. The method of any one of claims 1-38, wherein the condition mediated by the at least one integrin is PSC, and the compound inhibits the activity of at least one of α_{V}β₆ integrin and α_{V}β₁ integrin in in the subject effective to treat the subject for PSC.

40. The method of claim 39, wherein the compound inhibits the activity of α_{V}β₁ integrin and α_{V}β₆ integrin in the subject effective to treat the subject for PSC.

41. The method of any one of claims 1-40, wherein the condition mediated by the at least one integrin is psoriasis.

42. The method of claim 41, wherein the condition mediated by the at least one integrin is psoriasis, and the compound inhibits the activity of one or both of α_{V}β₁ integrin and α_{V}β₆ integrin in the subject effective to treat the subject for psoriasis.

43. The method of any one of claims 1-42, wherein the method selectively reduces α_{V}β₁ integrin activity and/or expression in the tissue compared to at least one other α_{V}-containing integrin in the subject.

44. The method of claim 43, wherein the method selectively reduces α_{V}β₁ integrin activity and/or expression in the tissue compared to α_{V}β₆ integrin in the subject.

45. The method of any one of claims 1-42, wherein the method selectively reduces α_{V}β₆ integrin activity and/or expression in the tissue compared to at least one other α_{V}-containing integrin in the subject.

46. The method of claim 45, wherein the method selectively reduces α_{V}β₆ integrin activity and/or expression in the tissue compared to α_{V}β₁ integrin in the subject.

47. The method of any one of claims 1-42, wherein the method selectively reduces α_{V}β₆ integrin or α_{V}β₁ integrin activity and/or expression in the tissue compared to at least one other α_{V}-containing integrin in the subject.

48. The method of any one of claims 1-42, wherein the method selectively reduces α_{V}β₆ integrin and α_{V}β₁ integrin activity and/or expression in the tissue compared to at least one other α_{V}-containing integrin in the subject.

49. The method of any one of claims 1-48, wherein the tissue comprises one or more fibroblasts, and the method inhibits α_{V}β₁ integrin in the one or more fibroblasts.

50. The method of any one of claims 1-49, wherein the tissue comprises one or more epithelial cells, and the method inhibits α_{V}β₆ integrin in the one or more epithelial cells.

51. The method of any one of claims 1-50, wherein a dose of the compound administered to the subject is an amount in milligrams selected from about one of, or at least about one of: 1, 2, 3, 4, 5, 10, 15, 20, 30, 40, 50, 60, 75, 80, 100, 120, 160, 240, 320, 400, 480, 560, 640, 720, 800, 880, 960, or 1040, or a range between any two of the preceding values.

52. The method of any one of claims 1-51, wherein a dose of the compound administered to the subject is an amount in milligrams selected from about one of, or at least about one of: 60, 120, 240, and 320, or a range between any two of the preceding values.

53. The method of any one of claims 1-52, comprising administering the compound to the subject effective to produce an unbound plasma concentration in nM of the compound in the subject selected from about one of, or at least about one of: 1, 10, 15, 20, 25, 30, 40, 50, 60, 75, 80, 90, 100, 125, 150, 200, 250, 500, 750, 1000, or 1250, or a range between any two of the preceding values.

54. The method of any one of claims 1-53, comprising administering the compound to the subject effective to produce an unbound plasma concentration in nM of the compound in the subject selected from about one of, or at least about one of: 10, 15, 20, 25, 30, 40, 50, 60, 75, 80, 90, 100, or 125, or a range between any two of the preceding values.

55. The method of any one of claims 1-54, wherein the compound is (S)-4-((2-methoxyethyl)(4-(5,6,7,8-tetrahydro-1,8-naphthyridin-2-yl)butyl)amino)-2-(quinazolin-4-ylamino)butanoic acid: 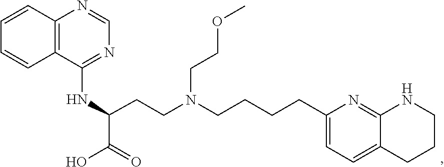 [SMILES: COCCN(CCCCc1ccc2c(n1)NCCC2)CC[C@H](Nc1ncnc2ccccc12)C(=O)O]
or a salt thereof.

56. The method of claim 55, wherein the compound is a phosphate, fumarate, 1,5-naphthalenedisulfonate, or a mixed solvate of isopropyl alcohol and water phosphate salt.

57. The method of claim 56, wherein the compound is a phosphate salt of Form I.

58. The method of claim 56, wherein the compound is a fumarate salt of Form II.

59. The method of claim 56, wherein the compound is a 1,5-naphthalenedisulfonate salt of Form III.

60. The method of claim 56, wherein the compound is a mixed solvate of isopropyl alcohol and water phosphate salt of Form IV.

61. The method of any one of claims 1-60, wherein the compound is administered to the subject in a dosage form configured for daily administration, the dosage form comprising a pharmaceutically acceptable carrier or excipient and a unit dose of the compound, or a salt thereof.

62. The method of claim 61, the dosage form comprising about 1, 2.5, 5, 7.5, 10, 15, 20, 25, 30, 35, 40, 50, 75, 80, 85, 90, 95, 100, 105, 110, 115, 120, or 125 mg of the compound, or a range between any two of the preceding values.

63. The method of claim 61, the dosage form comprising the compound in mg of about one of: 1, 2.5, 5, 7.5, 10, 15, 20, 25, 30, 35, 40, 50, 60, 75, 80, 85, 90, 95, 100, 105, 110, 115, 120, 125, 150, 175, 200, 225, or 250, or a range between any two of the preceding amounts.

64. The method of claim 61, the dosage form comprising the compound in mg of about one of: 10, 15, 20, 30, 40, 50, 75, 80, 100, 120, 160, 240, or 320, or a range between any two of the preceding values.

65. The method of claim 61, the dosage form comprising the compound in mg of about one of: 320, 400, 480, 560, 640, 720, 800, 880, 960, or 1040, or a range between any two of the preceding values.

66. The method of claim 61, the dosage form comprising the compound in an amount effective on administration to an individual to produce a Cₘₐₓ in plasma of the individual in ng/mL of at least about one of 700, 750, 800, 850, 900, 950, 1000, 1050, 1100, 1150, 1200, 1250, 1300, 1350, 1400, 1450, or 1500, or a range between any two of the preceding concentrations.

67. The method of claim 61, the dosage form comprising the compound in an amount effective on administration to an individual to produce a Cₘₐₓ in plasma of the individual in ng/mL of at least about one of: 1500, 1600, 1700, 1800, 1900, 2000, 2100, 2200, 2300, 2400, or 2500, or a range between any two of the preceding concentrations.

68. The method of claim 61, the dosage form comprising the compound in an amount effective on administration to an individual to produce a Cₘₐₓ in ng/mL in plasma of the individual, the Cₘₐₓ corresponding to a plasma-adjusted concentration effective to inhibit a percentage of α_{V}36 or α_{V}β₁ in the individual of at least about one of 50, 55, 60, 65, 70, 75, 80, 85, 90, 95, or 100, or a range between any two of the preceding percentages.

69. The method of claim 61, the dosage form configured for daily administration, the method comprising daily administration of the dosage form to the subject.

70. The method of claim 61, the dosage form configured for daily administration, the method comprising administration of the dosage form to the subject one, two, three, or four times daily.
